(19) United States
(12) Patent Application Publication
(10) Pub. No.: US 2024/0124466 A1
(43) Pub. Date: Apr. 18, 2024

(54) COMPOUNDS AND USE THEREOF FOR TREATMENT OF NEURODEGENERATIVE, DEGENERATIVE AND METABOLIC DISORDERS

(71) Applicants: VOVA IDA THERAPEUTICS, INC., Jupiter, FL (US); SCRIPPS RESEARCH INSTITUTE, THE, La Jolla, CA (US)

(72) Inventors: Thomas D. BANNISTER, Palm Beach Gardens, FL (US); Corinne LASMEZAS, Palm Beach Gardens, FL (US); Minghai ZHOU, Jupiter, FL (US); Nicole KENNEDY, West Palm Beach, FL (US)

(21) Appl. No.: 18/256,995

(22) PCT Filed: Dec. 10, 2021
(86) PCT No.: PCT/US21/62954

§ 371 Date: Jun. 12, 2023

Related U.S. Application Data
(60) Provisional application No. 63/124,543, filed on Dec. 11, 2020.

(51) Int. Cl. C07D 487/04; C07D 213/34; C07D 213/61; C07D 213/74; C07D 231/12; C07D 231/56; C07D 233/64; C07D 237/30; C07D 249/08; C07D 271/06; C07D 285/08; C07D 333/18; C07D 401/04; C07D 401/12; C07D 401/14; C07D 403/12; C07D 405/12; C07D 413/12; C07D 471/04; C07D 491/048; C07D 491/107; C07D 491/113; C07D 498/08

(52) U.S. Cl. CPC C07D 487/04; C07D 213/34; C07D 213/61; C07D 213/74; C07D 231/12; C07D 231/56; C07D 233/64; C07D 237/30; C07D 249/08; C07D 271/06; C07D 285/08; C07D 333/18; C07D 401/04; C07D 401/12; C07D 401/14; C07D 403/12; C07D 405/12; C07D 413/12; C07D 471/04; C07D 491/048; C07D 491/107; C07D 491/113; C07D 498/08

(57) ABSTRACT

Provided are, inter alia, compounds having a structure of Formula (X), or a pharmaceutically acceptable salt thereof composition including the same and methods of use. Ring A, L¹, L², W, R^{2A}, R^{2B}, and R¹⁰ are as described herein.

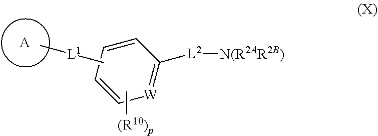

### CROSS-REFERENCES TO RELATED APPLICATIONS

This application claims the benefit of priority of U.S. Provisional Application No. 63/124,543 filed on Dec. 11, 2020, which is incorporated herein by reference in its entirety and for all purposes.

### BACKGROUND

A number of fatal neurodegenerative diseases, including prion diseases such as Creutzfeldt-Jakob disease (CJD), Alzheimer's (AD), Parkinson's (PD), frontotemporal dementia (FTD) and amyotrophic lateral sclerosis (ALS), are characterized by toxicity resulting from protein misfolding, and are called protein misfolding neurodegenerative diseases (PMNDs). Proteins involved in these diseases misfold and form aggregates of various sizes. Some of these aggregates are highly toxic for neurons, a phenomenon also referred to as proteotoxicity. Protein aggregates can also exhibit “prion-like” properties, in the sense that they propagate from cell to cell and act as seeds to amplify the misfolding and aggregation process within a cell. Such toxic misfolded proteins include the prion protein PrP in CJD, Aβ and tau in AD; α-synuclein and tau in PD; tau, TDP-43 and C90RF72 in FTD; SOD1, TDP43, FUS and C90RF72 in ALS. PD belongs to a broader group of diseases called synucleinopathies, characterized by the accumulation of misfolded α-synuclein aggregates. Lewy body dementia and Multiple System Atrophy are also synucleinopathies. FTD belongs to another group of PMNDs termed tauopathies, a group that also includes chronic traumatic encephalopathy (CTE) and progressive supranuclear palsy (PSP). There are also non-neurological diseases involving protein misfolding, such as diabetes mellitus where the proteins IAPP and proinsulin form protein aggregates that are toxic for pancreatic beta-cells, and cardiomyopathy caused by transthyretin (TTR) amyloidosis (ATTR). TTR amyloid deposits predominantly in peripheral nerves cause a polyneuropathy.

Poor knowledge of the mechanisms of neurotoxicity has hampered the development of effective therapies for PMNDs. To study such mechanisms, a model that uses misfolded and toxic prion protein (TPrP) has been developed, and in particular TPrP reproducibly induces neuronal death in cell culture and after intracerebral injection¹. TPrP induces death of more than 60% of cultured neurons at nanomolar concentration, whereas the natively folded counterpart of the prion protein, NTPrP, does not. Therefore, this model provides a highly efficient system to study mechanisms of neuronal death linked to proteotoxicity that are broadly applicable to protein misfolding diseases. Thus, as demonstrated herein, TPrP-based studies spurred the development of new neuroprotective approaches for treating devastating neurodegenerative diseases and other diseases involving the death of particular cell types.

### SUMMARY

Provided herein, inter alia, are novel compounds that may inhibit NAD consumption and/or increase NAD synthesis.

In an aspect, provided is a compound having a structure of Formula (X),

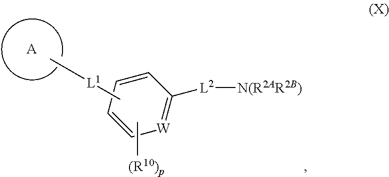

or a pharmaceutically acceptable salt thereof.

Ring A is a substituted or unsubstituted heteroaryl.

W is —CR¹═ or —N═.

L is a bond, substituted or unsubstituted alkylene, or substituted or unsubstituted heteroalkylene.

L² is —S(O)₂—, or —C(O)—.

R¹ is hydrogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl.

R¹⁰ is independently halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl.

p is an integer of 0 to 3.

X¹ is —F, —Br, —Cl, or —I.

R^{1A} is hydrogen, or substituted or unsubstituted alkyl.

Each R^{2A} and R^{2B} is independently selected from hydrogen, substituted or unsubstituted alkyl, substituted or unsubstituted heteroalkyl, substituted or unsubstituted cycloalkyl, or substituted or unsubstituted heterocycloalkyl; or R^{2A} and R^{2B} together with the nitrogen atom form a substituted or unsubstituted heterocycloalkyl, or substituted or unsubstituted heteroaryl.

In embodiments, the compound has a structure of Formula (XI) or (XI′),

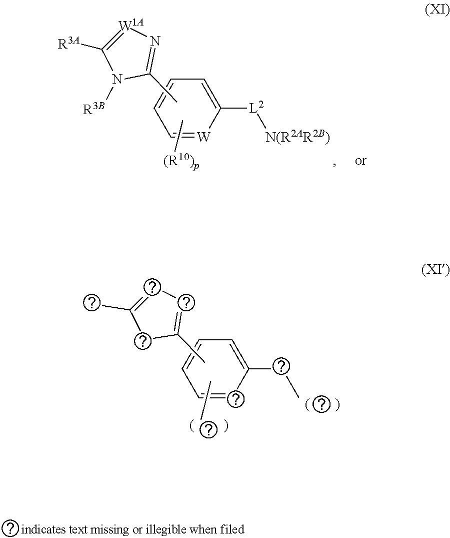

wherein:
W^{1A} is —N═, or —CR^{3C}═;
W^{1B} is —NH—, or —CR^{3A}R^{3C}—; and
Each R^{3A}, R^{3B}, and R^{3C} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
provided that when W^{1A} is —CR^{3C}═ and R^{3C} is hydrogen, then R^{2A} and R^{2B} together with the nitrogen atom form a substituted or unsubstituted heterocycloalkyl, or substituted or unsubstituted heteroaryl.

In embodiments, the compound has the structure of Formula (XII),

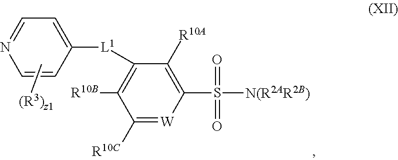

wherein:
L¹ is a bond or —NH—(CH₂)ₙ—;
n is an integer of 1 to 3;
z1 is an integer of 0 to 4;
R³ is independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

Each R^{10A}, R^{10B}, and R^{10C} is independently hydrogen, halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl.

In embodiments, the compound has the structure of Formula (XIII),

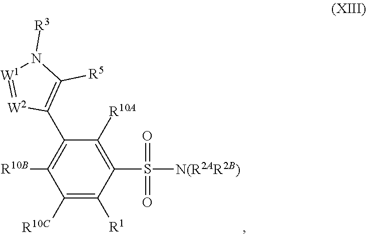

wherein:
W¹ is a —N═ or —CH═;
W² is a —N═ or —CR⁴—;

Each R³, R⁴ and R⁵ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

Each R^{10A}, R^{10B}, and R^{10C} is independently hydrogen, halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl;
provided that when R¹ is hydrogen, then R^{2A} and R^{2B} together with the nitrogen atom form a substituted or unsubstituted heterocycloalkyl, or substituted or unsubstituted heteroaryl.

In embodiments, the compound has the structure of Formula (XIV) or (XV),

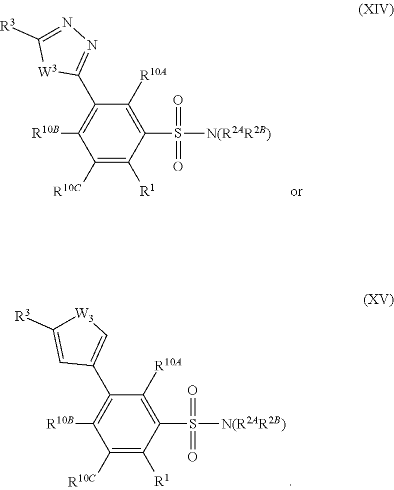

wherein:
W³ is a —S— or —O—;
R³ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

Each R^{10A}, R^{10B}, and R^{10C} is independently hydrogen, halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl.

In embodiments, the Ring A is bi-cyclic heteroaryl. In embodiments, the Ring A is selected from

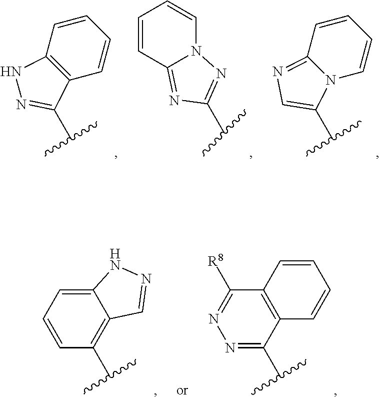

wherein R⁸ is hydrogen, or substituted or unsubstituted alkyl; the Ring A is unsubstituted or substituted with one or more R³, and R³ is independently halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. When Ring A is unsubstituted

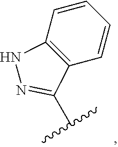 [SMILES: *c1n[nH]c2ccccc12]

—N(R^{2A}R^{2B}) is not a 4-substituted piperidinyl.

In an aspect, provided is a pharmaceutical composition including the compound described herein, a pharmaceutically acceptable salt form thereof, an isomer thereof, or a crystal form thereof.

In an aspect, provided is a method of inhibiting NAD consumption and/or increasing NAD synthesis in a patient. The method may include administering to the patient an effective dose of the compound as described herein.

In an aspect, provided is a method of preventing or inhibiting NAD depletion in a patient, or a method of improving a condition linked to alterations of NAD metabolism in a patient. The method may include administering to the patient an effective dose of the compound as described herein.

In an aspect, provided is a method of providing protection from toxicity of misfolded proteins in a patient. The method may include administering to the patient an effective dose of the compound as described herein.

In an aspect, provided is a method of preventing or treating a protein misfolding neurodegenerative disease in a patient. The method may include administering to the patient an effective dose of the compound as described herein.

In an aspect, provided is a method of preventing or treating retinal disease in a patient. The method may include administering to the patient an effective dose of the compound as described herein.

In an aspect, provided is a method of preventing or treating diabetes, non alcoholic fatty liver disease or other metabolic disease in a patient. The method may include administering to the patient an effective dose of the compound as described herein.

In an aspect, provided is a method of preventing or treating a kidney disease in a patient. The method may include administering to the patient an effective dose of the compound as described herein.

In an aspect, provided is a method of mitigating health effects of aging. The method may include administering to the patient an effective dose of the compound as described herein.

In an aspect, provided is a method of preventing or treating neuronal degeneration associated with multiple sclerosis, an axonopathy, a cardiomyopathy, brain or cardiac ischemia, traumatic brain injury, hearing loss, retinal damage, a metabolic disease, diabetes, non alcoholic fatty liver disease, or kidney failure in a patient. The method may include administering to the patient an effective dose of the compound as described herein.

Other aspects of the inventions are disclosed infra.

### BRIEF DESCRIPTION OF THE DRAWINGS

FIGS. 1A, 1B, 1C, 1D, 1E, 1F, 1G, 1H, 1I and 1J show dose-response curves of compounds in the TPrP neuroprotection assay.

FIGS. 2A and 2B show effects of compounds on the activation rate of the enzyme nicotinamide phosphoribosyltransferase (NAMPT).

### DETAILED DESCRIPTION

The misfolded toxic prion protein TPrP induces a profound depletion of neuronal NAD that is responsible for cell death, since NAD replenishment leads to full recovery of cells exposed to TPrP injury in vitro and in vivo, despite continued exposure to TPrP². Intranasal NAD treatment improved motor function and activity in murine prion disease. Further it was discovered that NAD depletion in neurons exposed to TPrP may be due, at least in part, to overconsumption of cellular NAD during metabolic reactions called mono-ADP ribosylations². Inhibitors of poly-ADP-ribosylations, called PARP inhibitors, have previously been developed as anticancer agents. Available selective PARP inhibitors did not alleviate NAD depletion and neuronal death caused by TPrP, demonstrating the need to identify new compounds capable of interfering with the mechanisms at play in misfolded protein-induced toxicity or capable of preventing NAD depletion irrespective of the mechanism underlying NAD imbalance. Imbalance in NAD metabolism is a pathogenic mechanism of a number of human conditions, as described herein.

NAD, as used here, designates both the oxidized (NAD+) and the reduced (NADH) forms of the cofactor. NAD is critical, inter alia, as a co-enzyme for the regulation of energy metabolism pathways such as glycolysis, TCA cycle and oxidative phosphorylation leading to ATP production. In addition, NAD serves as a substrate for signal transduction and post-translational protein modifications called ADP-ribosylations.

Physiological cellular NAD levels result from the balance of activity of NAD synthesis enzymes and NAD consuming enzymes, which may be reasoned that the NAD imbalance induced by misfolded proteins (and that is assessed in our TPrP assay) could therefore result from either impaired NAD biosynthesis or from increased NAD consumption.
In mammalian cells, NAD is mainly synthesized via the salvage pathway using the precursor nicotinamide (NAM). The rate-limiting enzyme for NAD synthesis in the salvage pathway is nicotinamide phosphoribosyltransferase (NAMPT). Other NAD synthesis pathways are the de novo pathway utilizing the precursor tryptophan and the Preiss-Handler pathway utilizing the precursor nicotinic acid (NA).
On the other hand, NAD is consumed during the following cellular reactions: 1) the production of calcium-releasing second messengers cyclic ADP-ribose (cADPR) and ADP-ribose (ADPR) from NAD by enzymes called NAD hydrolases or ADP-ribosyl cyclases (CD38 and CD157); 2) sirtuin-mediated protein deacetylations, and 3) protein ADP-ribosylations, in which one or several ADP-ribose moiety of NAD is transferred unto proteins by mono/oligo-ADP-ribose transferases (mARTs) or poly-ADP ribose transferases (called PARPs).

NAD deficiency is a feature of prion diseases² and other PMNDs such as PD^{3,4}, AD⁵⁻⁸ and ALS^{9,10}.

NAD dysregulation is now also recognized as being involved in aging¹¹⁻¹³, neuronal degeneration associated with multiple sclerosis¹⁴, traumatic brain injury¹⁵, hearing loss¹⁶, axonopathy and axonal degeneration^{17,18}. NAD augmentation such as NAD administration or increased NAD synthesis by enzyme overexpression has been shown to mitigate brain ischemia¹⁹ and cardiac ischemia/reperfusion injury^{20,21}.

Age-related retinal/macular degeneration (AMD) is associated with the death of photoreceptors and retinal pigment epithelium (RPE) cells of the eye's retina, and causes progressive loss of vision. NAD levels are decreased in RPE cells isolated from patients with AMD²². Healthy NAD levels are required for vision in mice²³. Increasing NAD levels by overexpression of cytoplasmic nicotinamide monomucleotide adenyl-transferase-1 (cytNMNAT1) in mice or NAM supplemented diet in rats showed less Zn²⁺ staining, NAD+ loss and cell death after light-induced retinal damage (LIRD)²⁴. Similarly, treatment with nicotinamide riboside (NR), a precursor of NAD, maintained retinal NAD levels and protected retinal morphology and function in a mouse model of LIRD²⁵.

NAD metabolism has also been shown to be altered in murine models of type 2 diabetes (T2D)^{26,27}. Alterations of NAD metabolism in diabetes can be explained, at least in part, by our findings that misfolded proteins induce NAD dysregulation. Indeed, diabetes has been shown to be a protein misfolding disease, characterized by pancreatic beta-cell dysfunction and death, concomitant with the deposition of aggregated islet amyloid polypeptide (IAPP), a protein co-expressed and secreted with insulin by pancreatic beta-cells^{28,29}. Similarly to proteins involved in other protein misfolding diseases, IAPP forms toxic oligomers²⁸. Moreover, proinsulin, the precursor of insulin, is also prone to misfold in beta-cells. Misfolding of proinsulin has been linked to type 2, type 1 and some monogenic forms of diabetes progression^{28,30,31}. NR supplementation mitigates type 2 diabetes in mice²⁷.

Substantial decreases in NAD levels are found in degenerative renal conditions and NAD augmentation mitigates acute kidney injury triggered by ischaemia-reperfusion, toxic injury and systemic inflammation³².

Using TPrP as a prototypic amyloidogenic misfolded protein exhibiting high neurotoxicity, a high-throughput screening (HTS) assay has been developed to identify compounds effective at a) preventing cell death; and b) preventing NAD depletion induced by TPrP.

The HTS campaign was performed at Scripps Florida using a subset of the Scripps Drug Discovery Library (SDDL). Several potent, novel and chemically tractable small molecules are identified that can provide complete neuroprotection and preservation of NAD levels when used at doses ranging from low nanomolar to low micromolar levels, which is also detailed in international patent Publication WO 2020/232255. Its entire content is incorporated herein by reference for all purposes.

Members of each series of compounds described herein are highly potent in neuroprotection assays designed to reflect the potential for the successful treatment of several neurodegenerative diseases as described herein. Several compounds described herein activate the NAD synthetic enzyme NAMPT. Further, many have favorable drug-like properties (e.g., they are PAINS-free³³ and compliant with Lipinski and Veber rules for drug-likenesss^{34,35}) Since these compounds prevent depletion of cellular NAD levels or increase NAD levels, they have utility in preventing or treating diseases where there is an imbalance in NAD metabolism, such as protein misfolding neurodegenerative diseases, amyloidoses, aging, retinal degeneration, ischemic conditions, traumatic brain injury, kidney failure and metabolic diseases including diabetes and non alcoholic fatty liver disease.

### Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di- and multivalent radicals. The alkyl may include a designated number of carbons (e.g., C₁-C₁₀ means one to ten carbons). Alkyl is an uncyclized chain. Examples of saturated hydrocarbon radicals include, but are not limited to, groups such as methyl (“Me”), ethyl (“Et”), n-propyl (“Pr”), isopropyl (“iPr”), n-butyl (“Bu”), t-butyl (“t-Bu”), isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkyl moiety may be fully saturated. An alkenyl may include more than one double bond and/or one or more triple bonds in addition to the one or more double bonds. An alkynyl may include more than one triple bond and/or one or more double bonds in addition to the one or more triple bonds.

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—S—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

In embodiments, a heterocycloalkyl is a heterocyclyl. The term “heterocyclyl” as used herein, means a monocyclic, bicyclic, or multicyclic heterocycle. The heterocyclyl monocyclic heterocycle is a 3, 4, 5, 6 or 7 membered ring containing at least one heteroatom independently selected from the group consisting of 0, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The heterocyclyl monocyclic heterocycle is connected to the parent molecular moiety through any carbon atom or any nitrogen atom contained within the heterocyclyl monocyclic heterocycle. Representative examples of heterocyclyl monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The heterocyclyl bicyclic heterocycle is a monocyclic heterocycle fused to either a phenyl, a monocyclic cycloalkyl, a monocyclic cycloalkenyl, a monocyclic heterocycle, or a monocyclic heteroaryl. The heterocyclyl bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. In embodiments, heterocyclyl groups are optionally substituted with one or two groups which are independently oxo or thia. In certain embodiments, the bicyclic heterocyclyl is a 5 or 6 membered monocyclic heterocyclyl ring fused to a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia. Multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a bicyclic aryl, a monocyclic or bicyclic heteroaryl, a monocyclic or bicyclic cycloalkyl, a monocyclic or bicyclic cycloalkenyl, and a monocyclic or bicyclic heterocyclyl. The multicyclic heterocyclyl is attached to the parent molecular moiety through any carbon atom or nitrogen atom contained within the base ring. In embodiments, multicyclic heterocyclyl ring systems are a monocyclic heterocyclyl ring (base ring) fused to either (i) one ring system selected from the group consisting of a bicyclic aryl, a bicyclic heteroaryl, a bicyclic cycloalkyl, a bicyclic cycloalkenyl, and a bicyclic heterocyclyl; or (ii) two other ring systems independently selected from the group consisting of a phenyl, a monocyclic heteroaryl, a monocyclic cycloalkyl, a monocyclic cycloalkenyl, and a monocyclic heterocyclyl. Examples of multicyclic heterocyclyl groups include, but are not limited to 10H-phenothiazin-10-yl, 9,10-dihydroacridin-9-yl, 9,10-dihydroacridin-10-yl, 10H-phenoxazin-10-yl, 10,11-dihydro-5H-dibenzo[b,f]azepin-5-yl, 1,2,3,4-tetrahydropyrido[4,3-g]isoquinolin-2-yl, 12H-benzo[b]phenoxazin-12-yl, and dodecahydro-1H-carbazol-9-yl.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substitutents described herein.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

The term “alkylsulfonyl,” as used herein, means a moiety having the formula —S(O₂)—R′, where R′ is a substituted or unsubstituted alkyl group as defined above. R′ may have a specified number of carbons (e.g., “C₁-C₄ alkylsulfonyl”).

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″, —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″, —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R″R′″)_{d}—, where s and d are independently integers of from 0 to 3, and X is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCl₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and (B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₅ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₅ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₅ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or —CH₃). Likewise, for a linker variable (e.g., L¹, L², or L³ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “pharmaceutically acceptable salts” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Thus, the compounds of the present disclosure may exist as salts, such as with pharmaceutically acceptable acids. The present disclosure includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g. methyl iodide, ethyl iodide, and the like). These salts may be prepared by methods known to those skilled in the art.

The neutral forms of the compounds are preferably regenerated by contacting the salt with a base or acid and isolating the parent compound in the conventional manner. The parent form of the compound may differ from the various salt forms in certain physical properties, such as solubility in polar solvents.

In addition to salt forms, the present disclosure provides compounds, which are in a prodrug form. Prodrugs of the compounds described herein are those compounds that readily undergo chemical changes under physiological conditions to provide the compounds of the present disclosure. Prodrugs of the compounds described herein may be converted in vivo after administration. Additionally, prodrugs can be converted to the compounds of the present disclosure by chemical or biochemical methods in an ex vivo environment, such as, for example, when contacted with a suitable enzyme or chemical reagent.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

“Pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure. One of skill in the art will recognize that other pharmaceutical excipients are useful in the present disclosure.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

As used herein, the term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “EC₅₀” or “half maximal effective concentration” as used herein refers to the concentration of a molecule (e.g., small molecule, drug, antibody, chimeric antigen receptor or bispecific antibody) capable of inducing a response which is halfway between the baseline response and the maximum response after a specified exposure time. In embodiments, the EC₅₀ is the concentration of a molecule (e.g., small molecule, drug, antibody, chimeric antigen receptor or bispecific antibody) that produces 50% of the maximal possible effect of that molecule.

As used herein, the term “neurodegenerative disorder” refers to a disease or condition in which the function of a subject's nervous system becomes impaired. Examples of neurodegenerative diseases that may be treated with a compound, pharmaceutical composition, or method described herein include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, chronic fatigue syndrome, Chronic Traumatic Encephalopathy, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, Kuru, Lewy body dementia, Machado-Joseph disease (Spinocerebellar ataxia type 3), Multiple sclerosis, Multiple System Atrophy, myalgic encephalomyelitis, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, progressive supranuclear palsy, or Tabes dorsalis.

As used herein, the term “retinal degeneration” refers to a disease or condition in which the vision of a subject becomes impaired due to dysfunction and/or damage of the eye's retina. Examples of retinal degeneration include age-related macular degeneration (AMD). Early stage AMD includes abnormalities of the retinal pigment epithelium and drusen. Late-stage AMD can include dry (non-neovascular, atrophic) macular degeneration, wet (neovascular) macular degeneration, proliferative diabetic retinopathy (PDR), diabetic macular edema (DME).

As used herein, the term “axonopathy” refers to functional or structural damage to a neuron or pheripheral nerve.

As used herein, the term “peripheral” refers to the part of the body anatomy located outside of the central nervous system.

As used herein, the term “amyloidosis” refers to a condition linked to the deposition of protein amyloid. An amyloidosis can occur in the central nervous system and is also referred to as a protein misfolding neurodegenerative disease (e.g. prion diseases, AD, PD and other synucleinopathies, ALS, tauopathies). An amyloidosis can occur outside of the central nervous system and can be widespread, i.e. systemic, or located in different organ systems. When amyloid deposits occurs in several organs, it is referred to as “multisystem”. Examples of amyloidoses are cardiomyopathy or polyneuropathy caused by the deposition of the protein TTR in the heart or peripheral nerves, respectively. Other examples of peripheral amyloidoses are AL (Primary) Amyloidosis or AA (Secondary) Amyloidosis.

As used herein, the term “metabolic disorder” refers to a disease or condition in which body metabolism, i.e. the process in which the body gets, makes and stores energy from food, is disrupted. Some metabolic disorders affect the breakdown of amino acids, carbohydrates, or lipids. Other metabolic disorders are known as mitochondrial diseases and affect mitochondria, the cellular organelles that produce energy. Examples of metabolic disorders are diabetes mellitus (sugar metabolism), hypercholesterolemia, Gaucher disease (lipid metabolism), non alcoholic fatty liver disease (NAFLD), metabolic syndrome (dyslipidemia, abdominal obesity, insulin resistance, proinflammatory state).

As used herein, the terms “kidney disease”, “kidney failure”, “renal disease” or “renal failure” refer to a disease or condition in which a subject loses kidney function. The condition can have various etiologies such as infectious, inflammatory, ischemic or traumatic. Kidney failure can be acute, leading to rapid loss of kidney function, or chronic, leading to gradual loss of kidney function. The condition ultimately leads to the accumulation of dangerous levels of fluid, electrolytes and waste products in the body. End-stage kidney failure is fatal without artificial filtering of the blood (dialysis) or kidney transplant.

As used herein, the term “ischemic condition” or “ischemia” refers to a condition in which the blood flow is restricted or reduced in a part of the body, such as the heart or the brain.

The terms “treating”, or “treatment” refers to any indicia of success in the therapy or amelioration of an injury, disease, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation. The term “treating” and conjugations thereof, may include prevention of an injury, pathology, condition, or disease. In embodiments, treating is preventing. In embodiments, treating does not include preventing.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

The term “prevent” refers to a decrease in the occurrence of disease symptoms in a patient. As indicated above, the prevention may be complete (no detectable symptoms) or partial, such that fewer symptoms are observed than would likely occur absent treatment.

“Patient” or “subject in need thereof” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, and other non-mammalian animals. In some embodiments, a patient is human.

A “effective amount” is an amount sufficient for a compound to accomplish a stated purpose relative to the absence of the compound (e.g. achieve the effect for which it is administered, treat a disease, reduce enzyme activity, increase enzyme activity, reduce a signaling pathway, or reduce one or more symptoms of a disease or condition). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

For any compound described herein, the therapeutically effective amount can be initially determined from cell culture assays. Target concentrations will be those concentrations of active compound(s) that are capable of achieving the methods described herein, as measured using the methods described herein or known in the art.

As is well known in the art, therapeutically effective amounts for use in humans can also be determined from animal models. For example, a dose for humans can be formulated to achieve a concentration that has been found to be effective in animals. The dosage in humans can be adjusted by monitoring compounds effectiveness and adjusting the dosage upwards or downwards, as described above. Adjusting the dose to achieve maximal efficacy in humans based on the methods described above and other methods is well within the capabilities of the ordinarily skilled artisan.

The term “therapeutically effective amount,” as used herein, refers to that amount of the therapeutic agent sufficient to ameliorate the disorder, as described above. For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a control.

Dosages may be varied depending upon the requirements of the patient and the compound being employed. The dose administered to a patient, in the context of the present disclosure, should be sufficient to effect a beneficial therapeutic response in the patient over time. The size of the dose also will be determined by the existence, nature, and extent of any adverse side-effects. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. Dosage amounts and intervals can be adjusted individually to provide levels of the administered compound effective for the particular clinical indication being treated. This will provide a therapeutic regimen that is commensurate with the severity of the individual's disease state.

As used herein, the term “administering” means oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. In embodiments, the administering does not include administration of any active agent other than the recited active agent.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

### Compounds

In an aspect, provided herein are compounds that may provide complete neuroprotection and protection of cell types other than neurons, and preservation of NAD levels. The compounds may be highly potent in a) preventing neuronal and/or cellular death; and b) preventing NAD depletion induced by TPrP, for example, as identified by neuroprotection assays when used at doses ranging from low nanomolar to low micromolar levels.

In an aspect, a compound has a Formula (I),

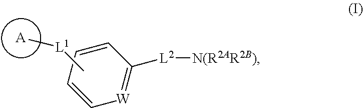

or a pharmaceutically acceptable salt thereof,
wherein in Formula (I):
Ring A is a substituted or unsubstituted heteroaryl,
W is —CR′═ or —N═;
L is a bond, substituted or unsubstituted alkylene, or substituted or unsubstituted heteroalkylene,
L² is —S(O)₂—, or —C(O)—;
R¹ is hydrogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl;
X¹ is —F, —Br, —Cl, or —I;
R^{1A} is hydrogen, or substituted or unsubstituted alkyl;
Each R^{2A} and R^{2B} is independently selected from hydrogen, substituted or unsubstituted alkyl, substituted or unsubstituted heteroalkyl, substituted or unsubstituted cycloalkyl, or substituted or unsubstituted heterocycloalkyl; or R^{2A} and R^{2B} together with the nitrogen atom form a substituted or unsubstituted heterocycloalkyl, or substituted or unsubstituted heteroaryl.

In an aspect, also provided is a compound having a structure of Formula (X),

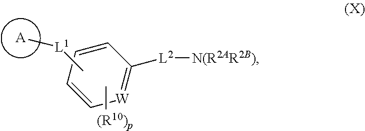

or a pharmaceutically acceptable salt thereof,
wherein in Formula (X):
Ring A is a substituted or unsubstituted heteroaryl,
W is —CR′═ or —N═;
L¹ is a bond, substituted or unsubstituted alkylene, or substituted or unsubstituted heteroalkylene;
L² is —S(O)₂—, or —C(O)—;
R¹ is hydrogen, —CX¹₃, —CHX₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl;
R¹⁰ is independently halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl;
   p is an integer of 0 to 3;
X¹ is —F, —Br, —Cl, or —I;
R^{1A} is hydrogen, or substituted or unsubstituted alkyl;

Each R^{2A} and R^{2B} is independently selected from hydrogen, substituted or unsubstituted alkyl, substituted or unsubstituted heteroalkyl, substituted or unsubstituted cycloalkyl, or substituted or unsubstituted heterocycloalkyl; or R^{2A} and R^{2B} together with the nitrogen atom form a substituted or unsubstituted heterocycloalkyl, or substituted or unsubstituted heteroaryl.

In embodiments, each R^{2A} and R^{2B} is independently selected from hydrogen, substituted or unsubstituted C₁-C₄ alkyl, substituted or unsubstituted C₆-C₁₂ cycloalkyl, or substituted or unsubstituted 4 to 12 membered heterocycloalkyl. In embodiments, R^{2A} is selected from hydrogen, substituted or unsubstituted C₁-C₄ alkyl, substituted or unsubstituted C₆-C₁₂ cycloalkyl, or substituted or unsubstituted 4 to 12 membered heterocycloalkyl (e.g., monocyclic, bicyclic, or multicyclic heterocyclic ring). In embodiments, R^{2A} is hydrogen. In embodiments, R^{2A} is substituted or unsubstituted C₁-C₄ alkyl. In embodiments, R^{2A} is substituted or unsubstituted C₆-C₁₂ cycloalkyl. In embodiments, R^{2A} is substituted or unsubstituted 4 to 12 membered heterocycloalkyl. In embodiments, R^{2B} is independently selected from hydrogen, substituted or unsubstituted C₁-C₄ alkyl, substituted or unsubstituted C₆-C₁₂ cycloalkyl, or substituted or unsubstituted 4 to 12 membered heterocycloalkyl. In embodiments, R^{2B} is hydrogen. In embodiments, R^{2B} is substituted or unsubstituted C₁-C₄ alkyl. In embodiments, R^{2B} is substituted or unsubstituted C₆-C₁₂ cycloalkyl. In embodiments, R^{2B} is substituted or unsubstituted 4 to 12 membered heterocycloalkyl.

In embodiments, R^{2A} and R^{2B} together with the nitrogen atom form a substituted or unsubstituted 4 to 12 membered heterocycloalkyl (e.g., monocyclic, bicyclic, or multicyclic heterocyclic ring), or a substituted or unsubstituted 5 to 12 membered heteroaryl (e.g., monocyclic, bicyclic, or multicyclic heterocyclic ring). In embodiments, R^{2A} and R^{2B} together with the nitrogen atom form a substituted or unsubstituted 4 to 12 membered heterocycloalkyl (e.g., monocyclic, bicyclic, or multicyclic heterocyclic ring). In embodiments, R^{2A} and R^{2B} together with the nitrogen atom form a substituted or unsubstituted 4 to 8 membered heterocycloalkyl (e.g., monocyclic, bicyclic, or multicyclic heterocyclic ring). In embodiments, R^{2A} and R^{2B} together with the nitrogen atom form a substituted or unsubstituted 4 to 6 membered heterocycloalkyl. In embodiments, R^{2A} and R^{2B} together with the nitrogen atom form a substituted or unsubstituted 4 to 5 membered heterocycloalkyl. In embodiments, R^{2A} and R^{2B} together with the nitrogen atom form a substituted or unsubstituted 5 to 6 membered heterocycloalkyl. In embodiments, R^{2A} and R^{2B} together with the nitrogen atom form a substituted or unsubstituted 5 to 12 membered heteroaryl. In embodiments, R^{2A} and R^{2B} together with the nitrogen atom form a substituted or unsubstituted 5 to 8 membered heteroaryl. In embodiments, R^{2A} and R^{2B} together with the nitrogen atom form a substituted or unsubstituted 5 to 6 membered heteroaryl.

In embodiments, L¹ is a bond, unsubstituted C₁-C₄ alkylene, or unsubstituted 2 to 4 membered heteroalkylene. In embodiments, L¹ is a bond. In embodiments, L¹ is unsubstituted C₁-C₄ alkylene. In embodiments, L¹ is unsubstituted methylene. In embodiments, L¹ is unsubstituted ethylene. In embodiments, L¹ is unsubstituted 2 to 4 membered heteroalkylene. In embodiments, L¹ is unsubstituted 2 to 4 membered heteroalkylene.

In embodiments, L¹ is —NH—(CH₂)ₙ— and n is an integer of 1 to 3. In embodiments, n is 1. In embodiments, n is 2. In embodiments, n is 3. In embodiments, L¹ is —NH—CH₂—.

In embodiments, the compound has a structure of Formula (II),

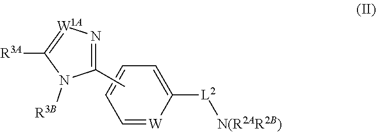

wherein:
W^{1A} is —N═, or —CR^{3C}═; and

Each R^{3A}, R^{3B}, and R^{3C} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. W, L², R^{2A}, and R^{2B} are as described herein.

In embodiments, the compound has a structure of Formula (XI),

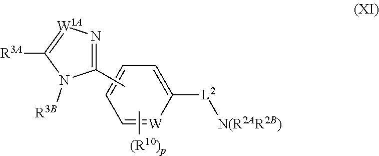

wherein:
W^{1A} is —N═, or —CR^{3C};

Each R^{3A}, R^{3B}, and R^{3C} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
provided that when W^{1A} is —CR^{3C}═ and R^{3C} is hydrogen, then R^{2A} and R^{2B} together with the nitrogen atom form a substituted or unsubstituted heterocycloalkyl, or substituted or unsubstituted heteroaryl.

In embodiments, W^{1A} is —N═. In embodiments, W^{1A} is —CR^{3C}═. In embodiments, the Ring A is

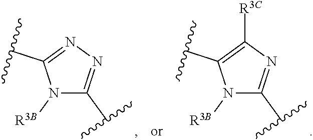

In embodiments, R^{3B} is hydrogen, or unsubstituted C₁-C₄ alkyl. In embodiments, R^{3B} is hydrogen. In embodiments, R^{3B} is —CH₃. In embodiments, Ring A is

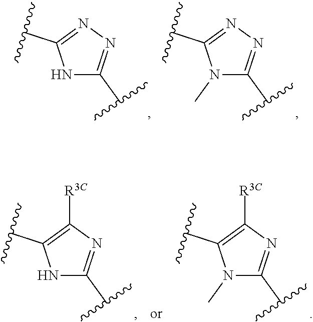

In embodiments, when R^{3C} is hydrogen, R^{2A} and R^{2B} together with the nitrogen atom form a substituted or unsubstituted morpholinyl.

In embodiments, the compound has a structure of Formula (II′),

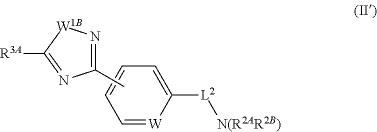

wherein:
W^{1B} is —NH—, or —CH₂—; and
R^{3A} is hydrogen, substituted or unsubstituted alkyl, a substituted or unsubstituted cycloalkyl, substituted or unsubstituted heterocycloalkyl, substituted or unsubstituted aryl, or substituted or unsubstituted heteroaryl. W, L², R^{2A}, and R^{2B} are as described herein.

In embodiments, the compound has a structure of Formula (XI′),

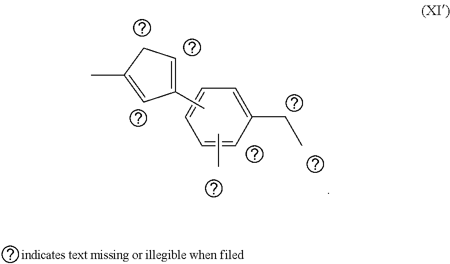

W, W^{1B}, L², R^{2A}, R^{2B}, R^{3A}, R¹⁰ and p are as described above.

In embodiments, W^{1B} is —NH—. In embodiments, W^{1B} is —CH₂—. In embodiments, the Ring A is

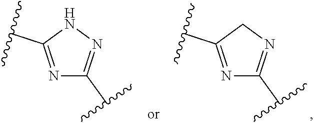 [SMILES: *c1n[nH]c(*)n1 ; *C1=NC(*)=NC1]

which may be substituted or unsubstituted.

In embodiments, R^{3A} is a substituted or unsubstituted C₅-C₆ cycloalkyl, substituted or unsubstituted phenyl, or substituted or unsubstituted pyridyl. In embodiments, R^{3A} is hydrogen.

In embodiments, R^{3A} is a substituted or unsubstituted C₅-C₆ cycloalkyl, substituted or unsubstituted phenyl, or substituted or unsubstituted pyridyl. In embodiments, R^{3A} is

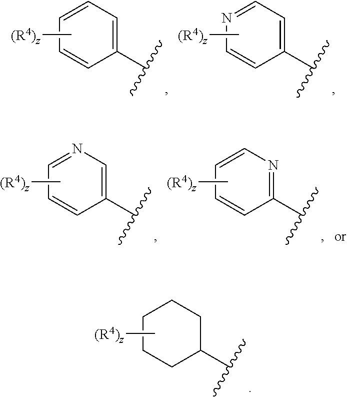

z is an integer of 0 to 5. In embodiments, z is 0. In embodiments, z is 2. In embodiments, z is 3. In embodiments, z is 4. In embodiments, z is 5.

Each R⁴ is independently halogen, —OR^{4A}, —NR^{4B}R^{4C}, —NO₂, substituted or unsubstituted alkyl, substituted or unsubstituted heteroalkyl; and each R^{4A}, R^{4B} and R^{4C} is independently hydrogen, or substituted or unsubstituted alkyl.

In embodiments, W is —CR¹═. In embodiments, W¹ is —N═.

In embodiments, the compound has the structure of Formula (II-a),

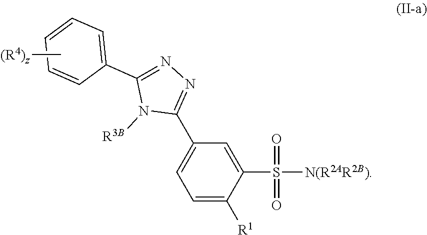

R¹, R^{2A}, R^{2B}, R^{3B}, R⁴ and z are as described herein.

In embodiments, the compound has the structure of Formula (XI-a).

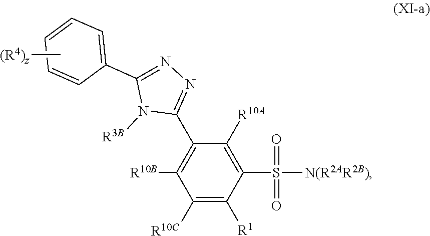

wherein:

Each R^{10A}, R^{10B}, and R^{10C} is independently hydrogen, halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl. R¹, R^{2A}, R^{2B}, R^{3B}, R⁴, and z are as described above.

In embodiments, Formula in (II-a) or (XI-a), R⁴ is —F, —Br, —OH, —OCH₃, —NH₂, —N(CH₃)₂, or NO₂. In embodiments, R⁴ is —F. In embodiments, R⁴ is —Br. In embodiments, R⁴ is —OH. In embodiments, R⁴ is —OCH₃. In embodiments, R⁴ is —NH₂. In embodiments, R⁴ is —N(CH₃)₂. In embodiments, R⁴ is —NO₂. In embodiments, z is 2 and both R⁴ are —F.

In embodiments, the compound has the structure of Formula (II′-a),

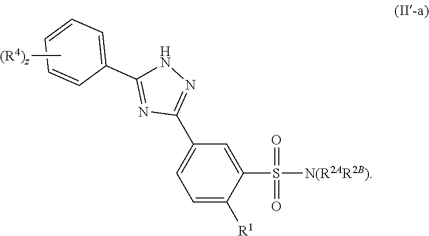

R¹, R^{2A}, R^{2B}, R⁴ and z are as described herein.

In embodiments, the compound has the structure of Formula (XI′-a)

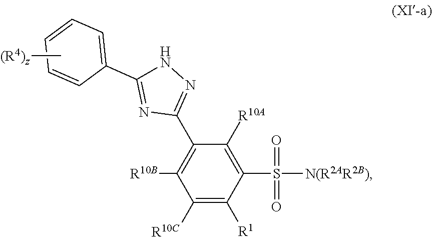

R¹, R^{2A}, R^{2B}, R⁴, R^{10A}, R^{10B}, R^{10C}, and z are as described herein.

In embodiments, the compound has the structure of Formula (II-b),

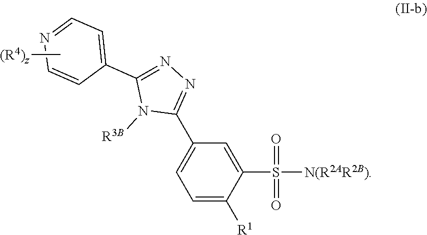

R¹, R^{2A}, R^{2B}, R^{3B}, R⁴ and z are as described herein.

In embodiments, Formula in (II-b), z is 0.

In embodiments, the compound has the structure of Formula (XI-b),

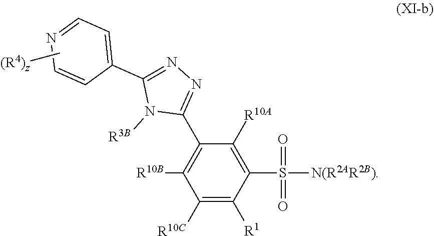

R¹, R^{2A}, R^{2B}, R^{3B}, R⁴, R^{10A}, R^{10B}, R^{10C}, and z are as described herein.

In embodiments, the compound has the structure of Formula (II-c),

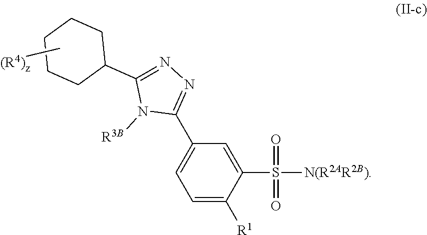

R¹, R^{2A}, R^{2B}, R^{3B}, R⁴ and z are as described herein.

In embodiments, the compound has the structure of Formula (XI-c),

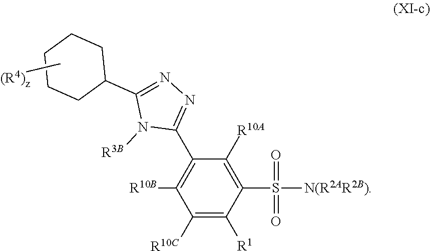

R¹, R^{2A}, R^{2B}, R^{3B}, R⁴, R^{10A}, R^{10B}, R^{10C}, and z are as described herein.

In embodiments, the compound has the structure of Formula (II-d),

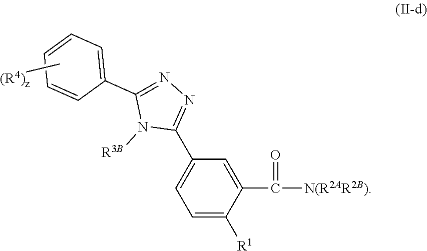

R¹, R^{2A}, R^{2B}, R^{3B}, R⁴ and z are as described herein.

In embodiments, the compound has the structure of Formula (XI-d),

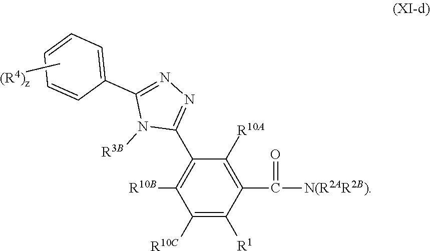

R¹, R^{2A}, R^{2B}, R^{3B}, R⁴, R^{10A}, R^{10B}, R^{10C}, and z are as described herein.

In embodiments, the compound has the structure of Formula (II-e),

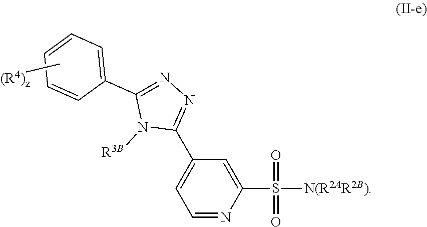

R¹, R^{2A}, R^{2B}, R^{3B}, R⁴ and z are as described herein.

In embodiments, the compound has the structure of Formula (XI-e),

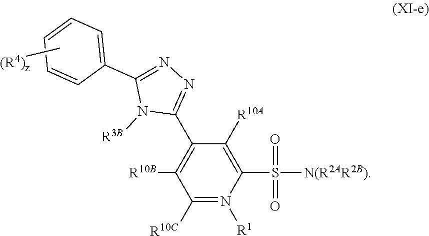

R¹, R^{2A}, R^{2B}, R^{3B}, R⁴, R^{10A}, R^{10B}, R^{10C}, and z are as described herein.

In embodiments, the compound has the structure of Formula (II-f),

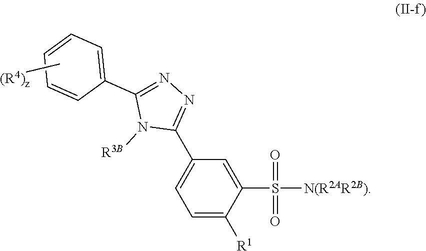

R¹, R^{2A}, R^{2B}, R^{3B}, R⁴ and z are as described herein.

In embodiments, the compound has the structure of Formula (XI-f),

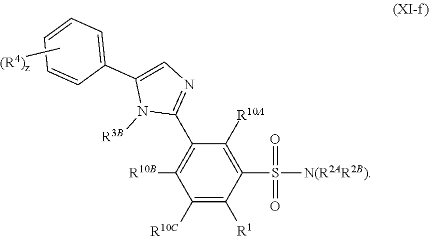

R¹, R^{2A}, R^{2B}, R^{3B}, R⁴, R^{10A}, R^{10B}, R^{10C}, and z are as described herein.

In embodiments, the compound has the structure of Formula (II-g),

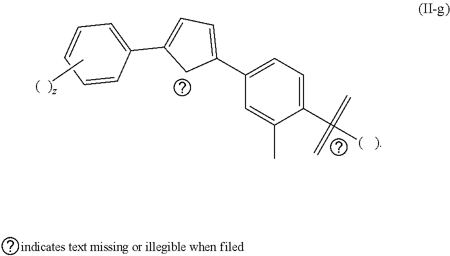

R¹, R^{2A}, R^{2B}, R^{3B}, R⁴ and z are as described herein.

In embodiments, the compound has the structure of Formula (XI-g),

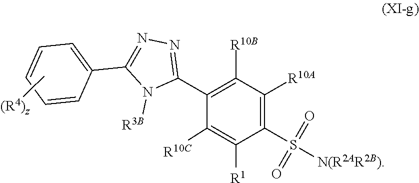

R¹, R^{2A}, R^{2B}, R^{3B}, R⁴, R^{10A}, R^{10B}, R^{10C}, and z are as described herein.

In embodiments, in Formula (II-a), (II′-a), (II-b), (II-c), (II-d), (II-e), (II-f), (II-g), (XI-a), (XI′-a), (XI-b), (XI-c), (XI-d), (XI-e), (XI-f), or (XI-g), z is an integer of 0 to 2. In embodiments, z is 0. In embodiments, z is 1. In embodiments, z is 2. In embodiments, in Formula (II′-a), (II-b), (II-c), (II-d), (II-e), (II-f), (II-g), (XI′-a), (XI-b), (XI-c), (XI-d), (XI-e), (XI-f), or (XI-g), z is 0. In embodiments, in Formula (II-a) or (XI-a), z is 0, 1, or 2.

In embodiments, in Formula (II-a), (II′-a), (II-b), (II-c), (II-d), (II-e), (II-f), (II-g), (XI-a), (XI′-a), (XI-b), (XI-c), (XI-d), (XI-e), (XI-f), or (XI-g), R^{3B} is hydrogen or —CH₃. In embodiments, R^{3B} is hydrogen. In embodiments, R^{3B} is —CH₃.

In embodiments, R^{2A} and R^{2B} together with the nitrogen attached thereto form a

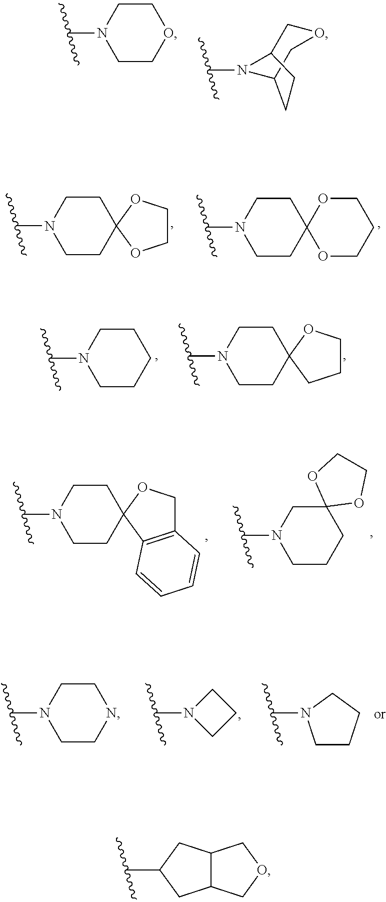

which is substituted or unsubstituted.

In embodiments, R^{2A} and R^{2B} together with the nitrogen attached thereto form a

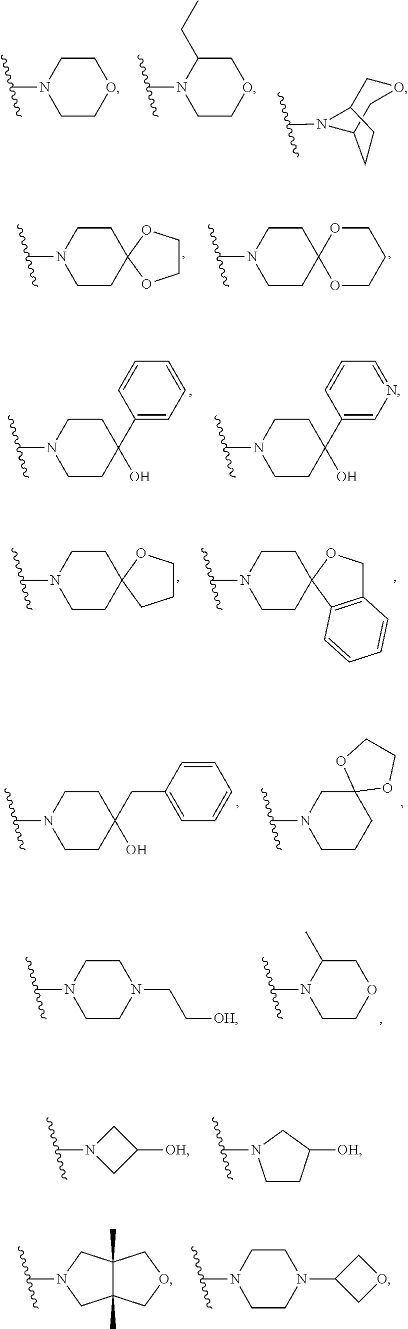

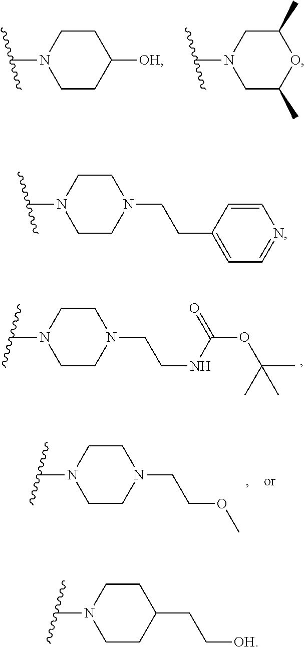

In embodiments, R^{2A} and R^{2B} together with the nitrogen attached thereto form

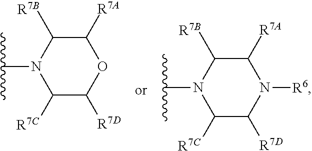

wherein R⁶ is hydrogen, halogen, —CX⁶₃, —CHX⁶₂, —CH₂X⁶, —OCX⁶₃, —OCH₂X⁶, —OCHX⁶₂, —CN, —OR^{6A}, substituted or unsubstituted alkyl, substituted or unsubstituted heteroalkyl, substituted or unsubstituted cycloalkyl, substituted or unsubstituted heterocycloalkyl;
X⁶ is —F, —Br, —Cl, or —I; and

Each R^{6A}, R^{7A}, R^{7B}, R^{7C} and R^{7D} is independently hydrogen, or substituted or unsubstituted alkyl.

In embodiments, R⁶ is —H,

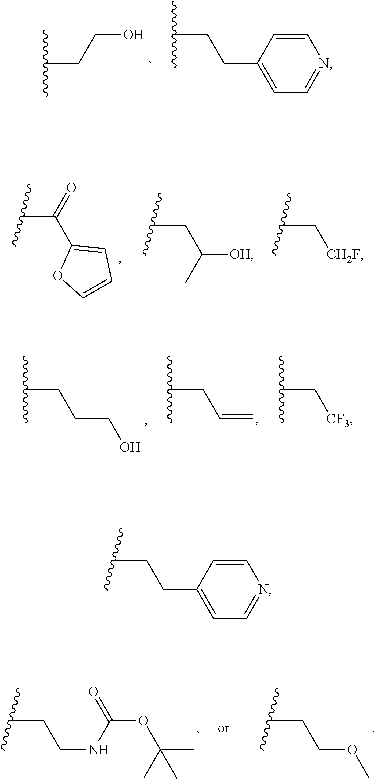

In embodiments, one of R^{2A} and R^{2B} is hydrogen and the other one of R^{2A} and R^{2B} is

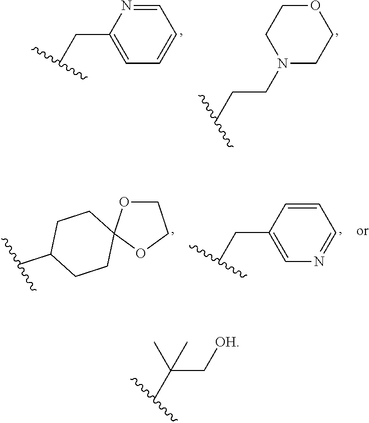

In embodiments, R¹ is —CH₃, —OCF₃, —CF₃, —OCH₃, —CN or

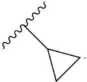 [SMILES: *C1CC1]

In embodiments, R¹ is —CH₃, —OCF₃, —CF₃, —OCH₃, —CN or

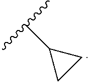 [SMILES: *C1CC1]

In embodiments, R¹ is —CH₃. In embodiments, R¹ is —OCF₃. In embodiments, R¹ is —CF₃. In embodiments, R¹ is —OCH₃. In embodiments, R¹ is —CN. In embodiments, R¹ is

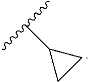 [SMILES: *C1CC1]

In embodiments, R¹ is not halogen. In embodiments, R¹ is not —Cl. In embodiments, R¹ is not —F.

In embodiments, each R^{10A}, R^{10B} and R^{10C} is independently hydrogen, halogen, or —CH₃. In embodiments, R^{10A} is hydrogen, halogen, or —CH₃. In embodiments, R^{10B} is hydrogen, halogen, or —CH₃. In embodiments, R^{10C} is hydrogen, halogen, or —CH₃.

In embodiments, R^{10A} is hydrogen. In embodiments, R^{10B} is hydrogen, —F, or —CH₃. In embodiments, R^{10C} is hydrogen, —F, or —CH₃.

In embodiment, in Formula (II-a) or (XI-a), R¹ is —CH₃, —OCF₃, —CF₃, —OCH₃, —CN or

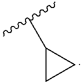 [SMILES: *C1CC1]

In embodiments, in Formula (II′-a), (II-c), (II-d), (II-e), (II-f), (XI′-a), (XI-c), (XI-d), (XI-e), or (XI-f), R¹ is —CH₃. In embodiment, in Formula (II-g) or (XI-g), R¹ is or —CF₃.

Exemplary compounds of Formula (II) or (XI) are shown in Table 1.

TABLE 1
Compound of Formula (II) or (XI)

| Compound | Structure |
|---|---|
| SR-25604 | 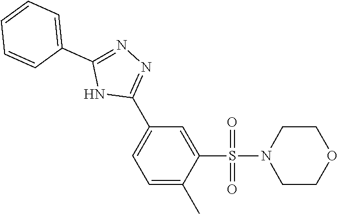 [SMILES: Cc1ccc(-c2nnc(-c3ccccc3)[nH]2)cc1S(=O)(=O)N1CCOCC1] |
| SR-32944 | 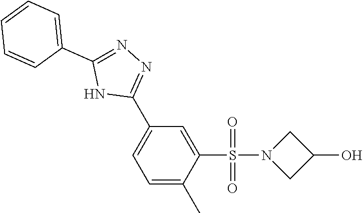 [SMILES: Cc1ccc(-c2nnc(-c3ccccc3)[nH]2)cc1S(=O)(=O)N1CC(O)C1] |
| SR-32945 | 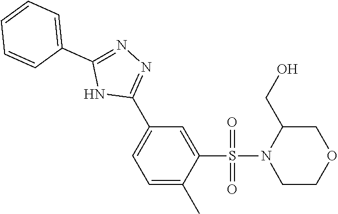 [SMILES: Cc1ccc(-c2nnc(-c3ccccc3)[nH]2)cc1S(=O)(=O)N1CCOCC1CO] |
| SR-32704 | 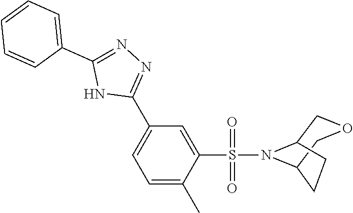 [SMILES: Cc1ccc(-c2nnc(-c3ccccc3)[nH]2)cc1S(=O)(=O)N1C2CCC1COC2] |
| SR-32144 | 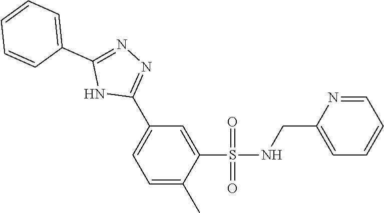 [SMILES: Cc1ccc(-c2nnc(-c3ccccc3)[nH]2)cc1S(=O)(=O)NCc1ccccn1] |
| SR-31824 | 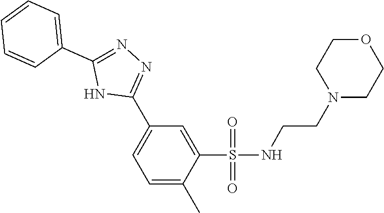 [SMILES: Cc1ccc(-c2nnc(-c3ccccc3)[nH]2)cc1S(=O)(=O)NCCN1CCOCC1] |
| SR-31545 | 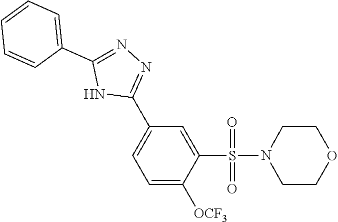 [SMILES: O=S(=O)(c1cc(-c2nnc(-c3ccccc3)[nH]2)ccc1OC(F)(F)F)N1CCOCC1] |
| SR-29045 | 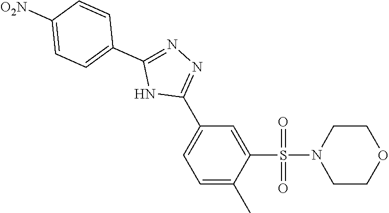 [SMILES: Cc1ccc(-c2nnc(-c3ccc([N+](=O)[O-])cc3)[nH]2)cc1S(=O)(=O)N1CCOCC1] |
| SR-32705 | 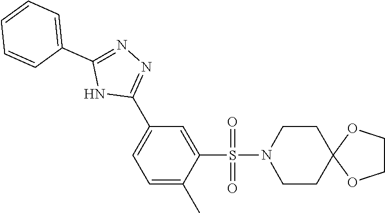 [SMILES: Cc1ccc(-c2nnc(-c3ccccc3)[nH]2)cc1S(=O)(=O)N1CCC2(CC1)OCCO2] |
| SR-32744 | 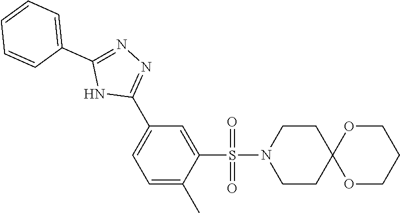 [SMILES: Cc1ccc(-c2nnc(-c3ccccc3)[nH]2)cc1S(=O)(=O)N1CCC2(CC1)OCCCO2] |
| SR-32764 | 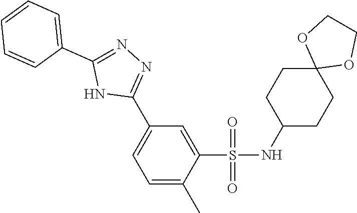 [SMILES: Cc1ccc(-c2nnc(-c3ccccc3)[nH]2)cc1S(=O)(=O)NC1CCC2(CC1)OCCO2] |
| SR-32784 | 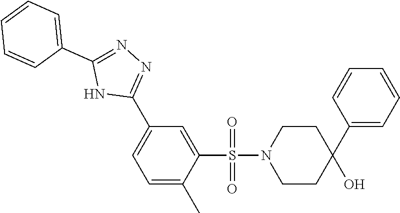 [SMILES: Cc1ccc(-c2nnc(-c3ccccc3)[nH]2)cc1S(=O)(=O)N1CCC(O)(c2ccccc2)CC1] |
| SR-32924 | 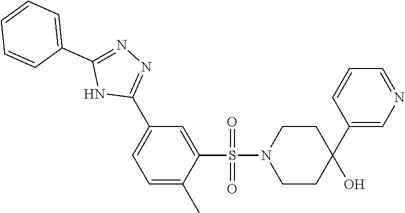 [SMILES: Cc1ccc(-c2nnc(-c3ccccc3)[nH]2)cc1S(=O)(=O)N1CCC(O)(c2cccnc2)CC1] |
| SR-29784 | 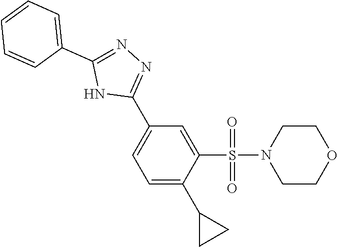 [SMILES: O=S(=O)(c1cc(-c2nnc(-c3ccccc3)[nH]2)ccc1C1CC1)N1CCOCC1] |
| SR-30786 | 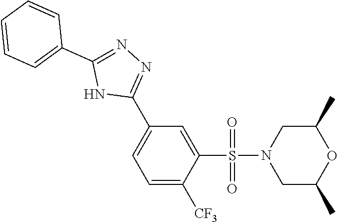 [SMILES: C[C@@H]1CN(S(=O)(=O)c2cc(-c3nnc(-c4ccccc4)[nH]3)ccc2C(F)(F)F)C[C@H](C)O1] |
| SR-28224 | 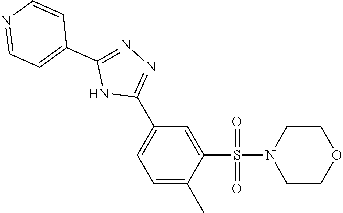 [SMILES: Cc1ccc(-c2nnc(-c3ccncc3)[nH]2)cc1S(=O)(=O)N1CCOCC1] |
| SR-28565 | 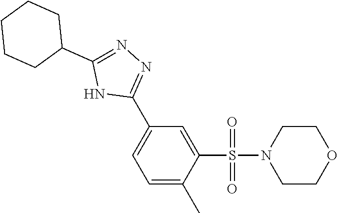 [SMILES: Cc1ccc(-c2nnc(C3CCCCC3)[nH]2)cc1S(=O)(=O)N1CCOCC1] |
| SR-28864 | 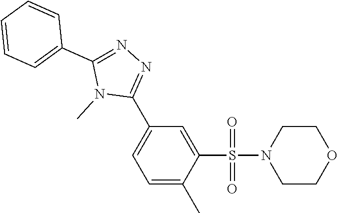 [SMILES: Cc1ccc(-c2nnc(-c3ccccc3)n2C)cc1S(=O)(=O)N1CCOCC1] |
| SR-32925 | 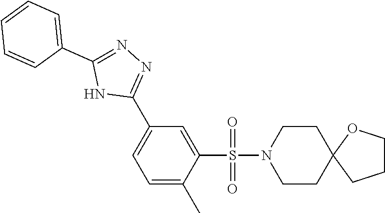 [SMILES: Cc1ccc(-c2nnc(-c3ccccc3)[nH]2)cc1S(=O)(=O)N1CCC2(CCCO2)CC1] |
| SR-32984 | 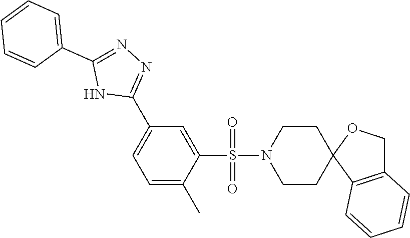 [SMILES: Cc1ccc(-c2nnc(-c3ccccc3)[nH]2)cc1S(=O)(=O)N1CCC2(CC1)OCc1ccccc12] |
| SR-33024 | 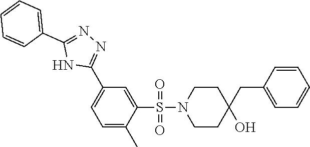 [SMILES: Cc1ccc(-c2nnc(-c3ccccc3)[nH]2)cc1S(=O)(=O)N1CCC(O)(Cc2ccccc2)CC1] |
| SR-33045 | 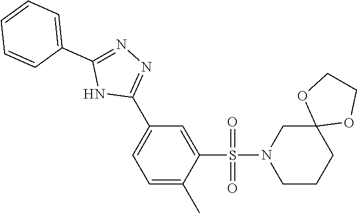 [SMILES: Cc1ccc(-c2nnc(-c3ccccc3)[nH]2)cc1S(=O)(=O)N1CCCC2(C1)OCCO2] |
| SR-32124 | 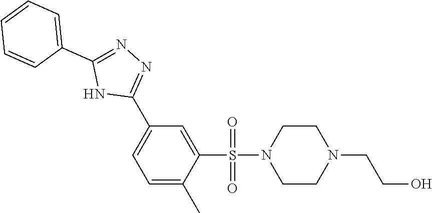 [SMILES: Cc1ccc(-c2nnc(-c3ccccc3)[nH]2)cc1S(=O)(=O)N1CCN(CCO)CC1] |
| SR-28124 | 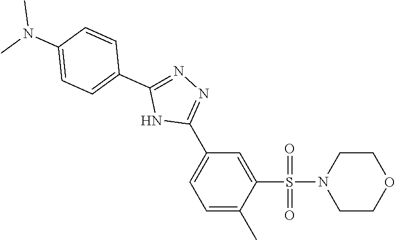 [SMILES: Cc1ccc(-c2nnc(-c3ccc(N(C)C)cc3)[nH]2)cc1S(=O)(=O)N1CCOCC1] |
| SR-28104 | 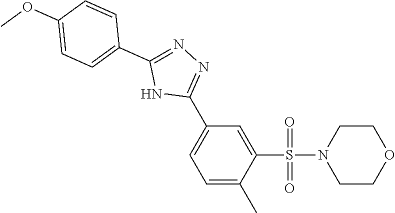 [SMILES: COc1ccc(-c2nnc(-c3ccc(C)c(S(=O)(=O)N4CCOCC4)c3)[nH]2)cc1] |
| SR-27984 | 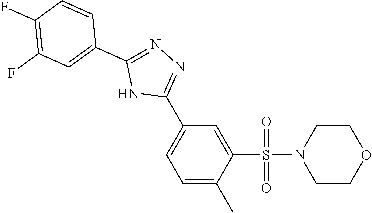 [SMILES: Cc1ccc(-c2nnc(-c3ccc(F)c(F)c3)[nH]2)cc1S(=O)(=O)N1CCOCC1] |
| SR-28125 | 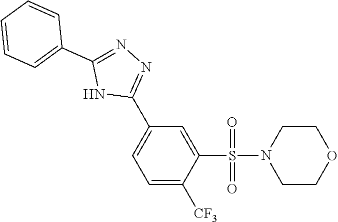 [SMILES: O=S(=O)(c1cc(-c2nnc(-c3ccccc3)[nH]2)ccc1C(F)(F)F)N1CCOCC1] |
| SR-27824 | 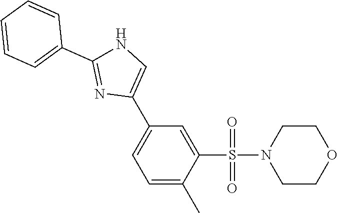 [SMILES: Cc1ccc(-c2c[nH]c(-c3ccccc3)n2)cc1S(=O)(=O)N1CCOCC1] |
| SR-31584 | 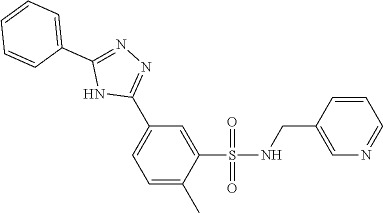 [SMILES: Cc1ccc(-c2nnc(-c3ccccc3)[nH]2)cc1S(=O)(=O)NCc1cccnc1] |
| SR-32286 | 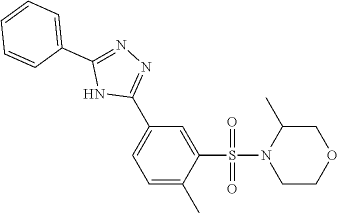 [SMILES: Cc1ccc(-c2nnc(-c3ccccc3)[nH]2)cc1S(=O)(=O)N1CCOCC1C] |
| SR-32184 | 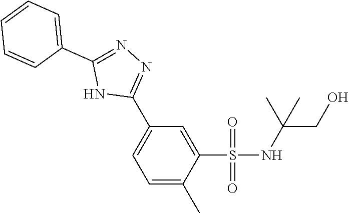 [SMILES: Cc1ccc(-c2nnc(-c3ccccc3)[nH]2)cc1S(=O)(=O)NC(C)(C)CO] |
| SR-32284 | 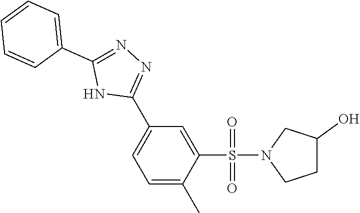 [SMILES: Cc1ccc(-c2nnc(-c3ccccc3)[nH]2)cc1S(=O)(=O)N1CCC(O)C1] |
| SR-32404 | 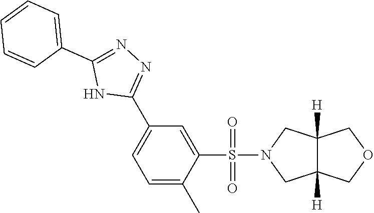 [SMILES: Cc1ccc(-c2nnc(-c3ccccc3)[nH]2)cc1S(=O)(=O)N1C[C@H]2COC[C@H]2C1] |
| SR-28144 | 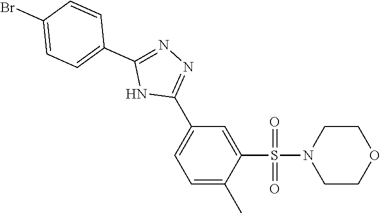 [SMILES: Cc1ccc(-c2nnc(-c3ccc(Br)cc3)[nH]2)cc1S(=O)(=O)N1CCOCC1] |
| SR-27964 | 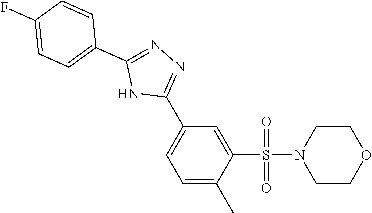 [SMILES: Cc1ccc(-c2nnc(-c3ccc(F)cc3)[nH]2)cc1S(=O)(=O)N1CCOCC1] |
| SR-28004 | 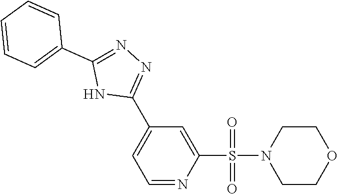 [SMILES: O=S(=O)(c1cc(-c2nnc(-c3ccccc3)[nH]2)ccn1)N1CCOCC1] |
| SR-25604-2 | 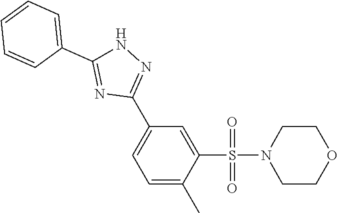 [SMILES: Cc1ccc(-c2n[nH]c(-c3ccccc3)n2)cc1S(=O)(=O)N1CCOCC1] |
| SR-26525 | 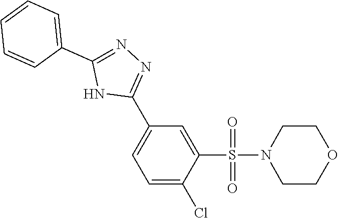 [SMILES: O=S(=O)(c1cc(-c2nnc(-c3ccccc3)[nH]2)ccc1Cl)N1CCOCC1] |
| SR-32324 | 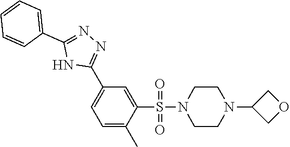 [SMILES: Cc1ccc(-c2nnc(-c3ccccc3)[nH]2)cc1S(=O)(=O)N1CCN(C2COC2)CC1] |
| SR-32044 | 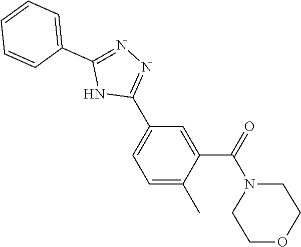 [SMILES: Cc1ccc(-c2nnc(-c3ccccc3)[nH]2)cc1C(=O)N1CCOCC1] |
| SR-32045 | 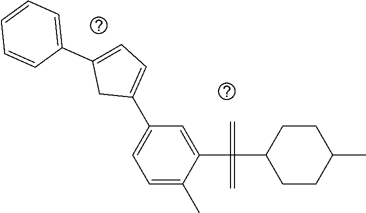 |
| SR-26324 | 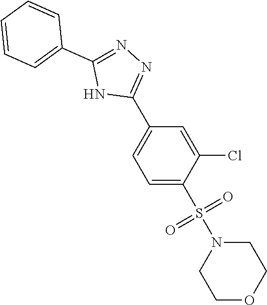 [SMILES: O=S(=O)(c1ccc(-c2nnc(-c3ccccc3)[nH]2)cc1Cl)N1CCOCC1] |
| SR-34778 | 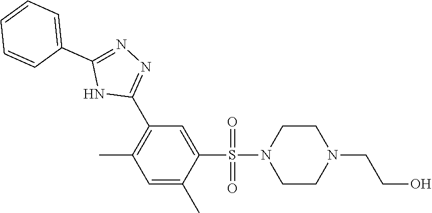 [SMILES: Cc1cc(C)c(S(=O)(=O)N2CCN(CCO)CC2)cc1-c1nnc(-c2ccccc2)[nH]1] |
| SR-26624 | 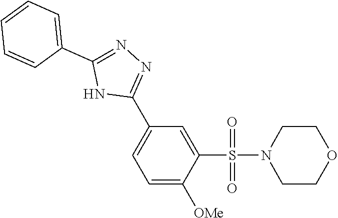 [SMILES: COc1ccc(-c2nnc(-c3ccccc3)[nH]2)cc1S(=O)(=O)N1CCOCC1] |
| SR-27504 | 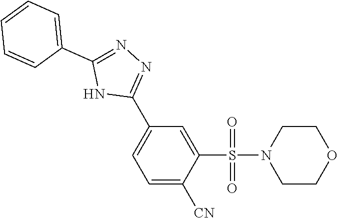 [SMILES: N#Cc1ccc(-c2nnc(-c3ccccc3)[nH]2)cc1S(=O)(=O)N1CCOCC1] |
| SR-28804 | 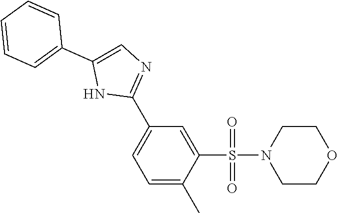 [SMILES: Cc1ccc(-c2ncc(-c3ccccc3)[nH]2)cc1S(=O)(=O)N1CCOCC1] |
| SR-26444 | 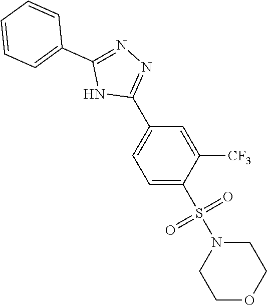 [SMILES: O=S(=O)(c1ccc(-c2nnc(-c3ccccc3)[nH]2)cc1C(F)(F)F)N1CCOCC1] |
| SR-33364 | 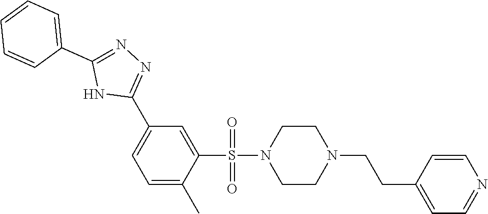 [SMILES: Cc1ccc(-c2nnc(-c3ccccc3)[nH]2)cc1S(=O)(=O)N1CCN(CCc2ccncc2)CC1] |
| SR-33604 | 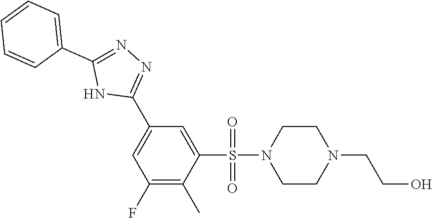 [SMILES: Cc1c(F)cc(-c2nnc(-c3ccccc3)[nH]2)cc1S(=O)(=O)N1CCN(CCO)CC1] |
| SR-34024 | 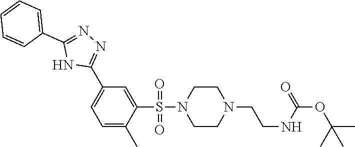 [SMILES: Cc1ccc(-c2nnc(-c3ccccc3)[nH]2)cc1S(=O)(=O)N1CCN(CCNC(=O)OC(C)(C)C)CC1] |
| SR-33725 | 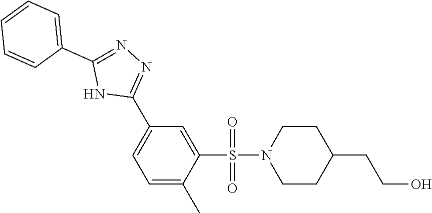 [SMILES: Cc1ccc(-c2nnc(-c3ccccc3)[nH]2)cc1S(=O)(=O)N1CCC(CCO)CC1] |
| SR-33344 | 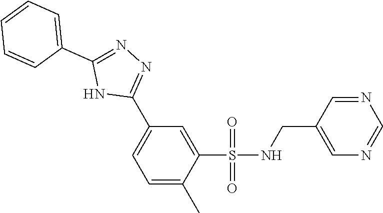 [SMILES: Cc1ccc(-c2nnc(-c3ccccc3)[nH]2)cc1S(=O)(=O)NCc1cncnc1] |
| SR-34533 | 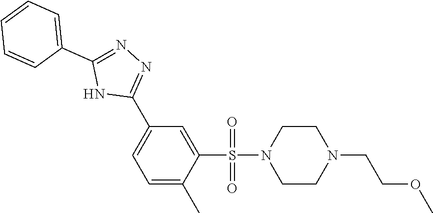 [SMILES: COCCN1CCN(S(=O)(=O)c2cc(-c3nnc(-c4ccccc4)[nH]3)ccc2C)CC1] |

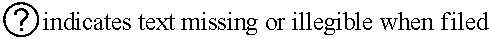

In embodiments, the compound has the structure of Formula (III),

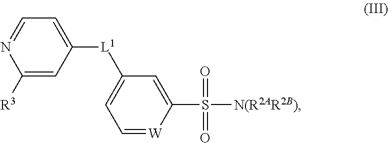

wherein:
L is a bond or —NH—(CH₂)ₙ—;
n is an integer of 1 to 3,
R³ is independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. W, R^{2A}, and R^{2B} are as described herein.

In embodiments, the compound has the structure of Formula (XII),

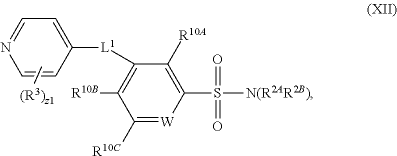

wherein:
L is a bond or —NH—(CH₂)ₙ—;
n is an integer of 1 to 3;
z1 is an integer of 0 to 4;
R³ is independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and

Each R^{10A}, R^{10B}, and R^{10C} is independently hydrogen, halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl. W, R^{2A}, and R^{2B} are as described herein.

In embodiments, R^{2A} and R^{2B} together with the nitrogen attached thereto form

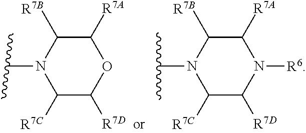

R⁶, R^{7A}, R^{7B}, R^{7C} and R^{7D} are as described herein.

In embodiments, R^{2A} and R^{2B} together with the nitrogen attached thereto form

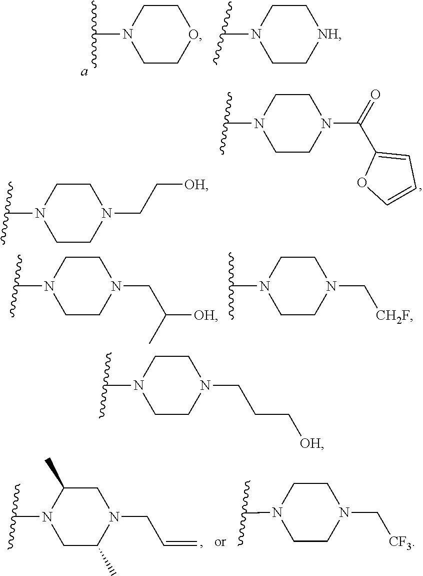

In embodiments, the compound has the structure of Formula (III-a),

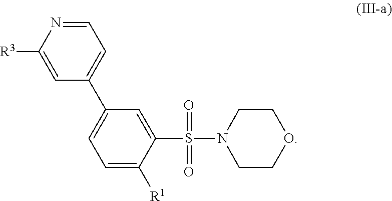

R¹ and R³ are as described herein.

In embodiments, the compound has the structure of Formula (III-b),

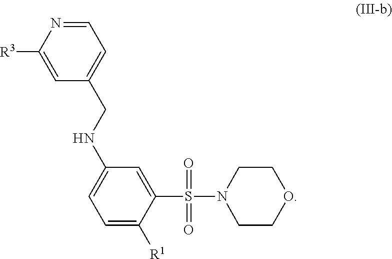

R¹ and R³ are as described herein.

In embodiments, the compound has the structure of Formula (XII-a),

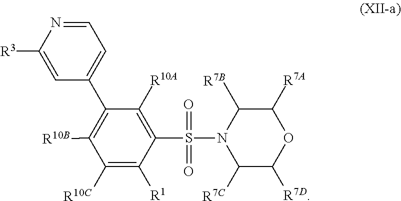

R¹, R³, R^{7A}, R^{7B}, R^{7C}, R^{7D}, R^{10A}, R^{10B}, and R^{10C} are as described herein.

In embodiments, the compound has the structure of Formula (XII-b),

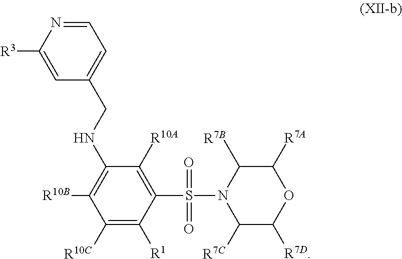

R¹, R³, R^{7A}, R^{7B}, R^{7C}, R^{7D}, R^{10A}, R^{10B}, and R^{10C} are as described herein.

In embodiments, the compound has the structure of Formula (XII-c),

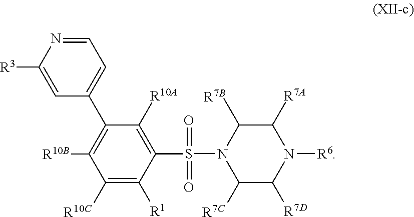

R¹, R³, R^{7A}, R^{7B}, R^{7C}, R^{7D}, R^{10A}, R^{10B}, and R^{10C} are as described herein.

In embodiments, R⁶ is —H,

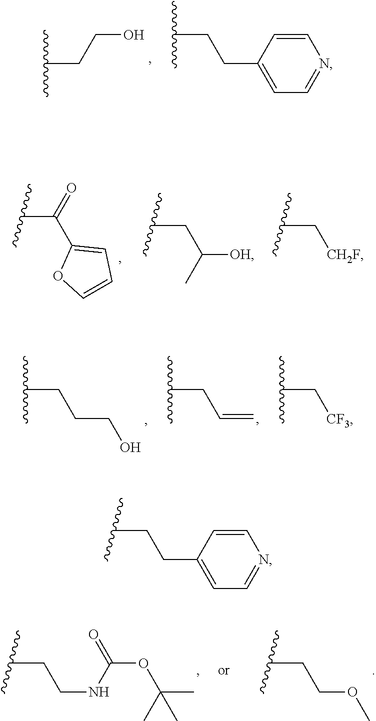

In embodiments, each R^{6A}, R^{7A}, R^{7B}, R^{7C} and R^{7D} is independently hydrogen, or —CH₃.

In embodiments, two of R^{7A}, R^{7B}, R^{7C} and R^{7D} are independently hydrogen and the other two are —CH₃. In embodiments, R^{7A} and R^{7C} are hydrogen, and R^{7B} and R^{7D} are —CH₃. In embodiments, R^{7A} and R^{7C} are —CH₃, and R^{7B} and R^{7D} are hydrogen. In embodiments, R^{7A} and R^{7D} are hydrogen, and R^{7B} and R^{7C} are —CH₃. In embodiments, R^{7A} and R^{7D} are —CH₃, and R^{7B} and R^{7C} are hydrogen.

In embodiments, in Formula (III-a), (III-b), (XII-a) or (XII-b), R¹ is —CH₃.

In embodiments, R³ is hydrogen, halogen, substituted unsubstituted pyridyl, substituted or unsubstituted morphorinyl, substituted or unsubstituted phenyl, substituted or unsubstituted 2-6 membered heteroalkyl. In embodiments, R³ is hydrogen. In embodiments, R³ is a substituted unsubstituted pyridyl. In embodiments, R³ is a substituted or unsubstituted morphorinyl. In embodiments, R³ is a substituted or unsubstituted phenyl. In embodiments, R³ is a substituted or unsubstituted 2-6 membered heteroalkyl.

In embodiments, R³ is hydrogen, halogen,

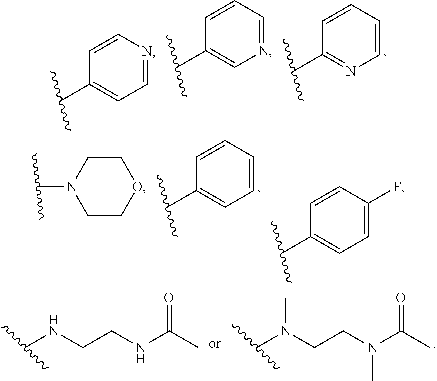

In embodiments, each R^{10A}, R^{10B} and R^{10C} is independently hydrogen, halogen, or —CH₃. In embodiments, R^{10A} is hydrogen, halogen, or —CH₃. In embodiments, R^{10B} is hydrogen, halogen, or —CH₃. In embodiments, R^{10C} is hydrogen, halogen, or —CH₃.

In embodiments, R^{10A} is hydrogen. In embodiments, R^{10B} is hydrogen, —F, or —CH₃. In embodiments, R^{10C} is hydrogen, —F, or —CH₃.

Exemplary compounds of Formula (III) or (XII) are shown in Table 2.

TABLE 2
Compound of Formula (III) or (XII)

| Com- | |
|---|---|
| pound | Structure |
| SR- 29204 | 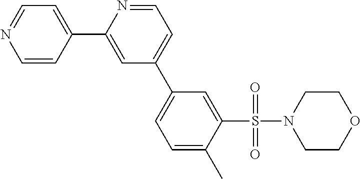 [SMILES: Cc1ccc(-c2ccnc(-c3ccncc3)c2)cc1S(=O)(=O)N1CCOCC1] |
| SR- 28984 | 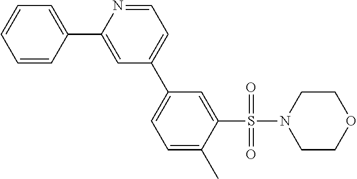 [SMILES: Cc1ccc(-c2ccnc(-c3ccccc3)c2)cc1S(=O)(=O)N1CCOCC1] |
| SR- 25104 | 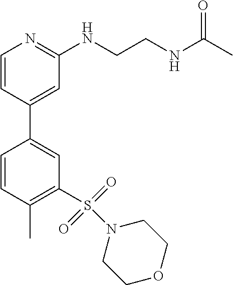 [SMILES: CC(=O)NCCNc1cc(-c2ccc(C)c(S(=O)(=O)N3CCOCC3)c2)ccn1] |
| SR- 25464 | 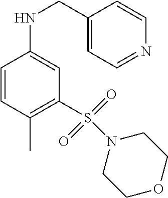 [SMILES: Cc1ccc(NCc2ccncc2)cc1S(=O)(=O)N1CCOCC1] |
| SR- 29224 | 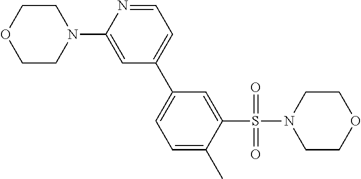 [SMILES: Cc1ccc(-c2ccnc(N3CCOCC3)c2)cc1S(=O)(=O)N1CCOCC1] |
| SR- 28924 | 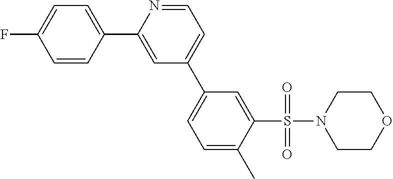 [SMILES: Cc1ccc(-c2ccnc(-c3ccc(F)cc3)c2)cc1S(=O)(=O)N1CCOCC1] |
| SR- 25124 | 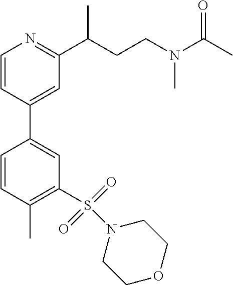 [SMILES: CC(=O)N(C)CCC(C)c1cc(-c2ccc(C)c(S(=O)(=O)N3CCOCC3)c2)ccn1] |
| SR- 24583 | 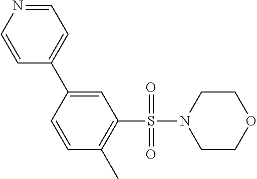 [SMILES: Cc1ccc(-c2ccncc2)cc1S(=O)(=O)N1CCOCC1] |
| SR- 34793 | 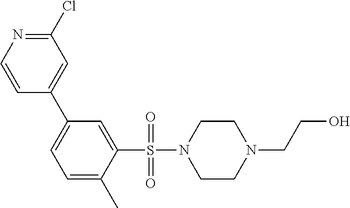 [SMILES: Cc1ccc(-c2ccnc(Cl)c2)cc1S(=O)(=O)N1CCN(CCO)CC1] |
| SR- 35015 | 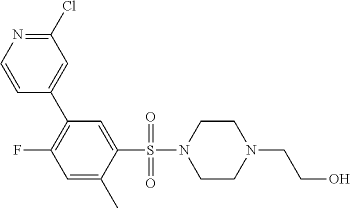 [SMILES: Cc1cc(F)c(-c2ccnc(Cl)c2)cc1S(=O)(=O)N1CCN(CCO)CC1] |
| SR- 35516 | 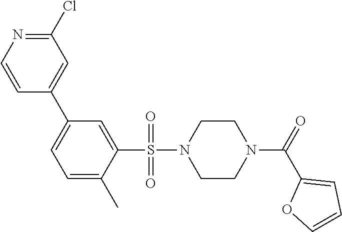 [SMILES: Cc1ccc(-c2ccnc(Cl)c2)cc1S(=O)(=O)N1CCN(C(=O)c2ccco2)CC1] |
| SR- 35017 | 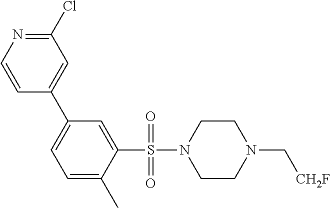 |
| SR- 35124 | 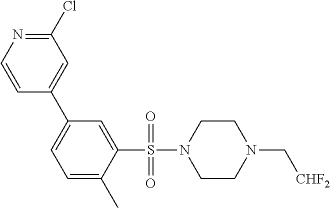 [SMILES: Cc1ccc(-c2ccnc(Cl)c2)cc1S(=O)(=O)N1CCN(CC(F)F)CC1] |
| SR- 35422 | 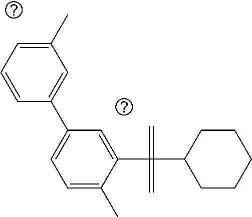 |
| SR- 34951 | 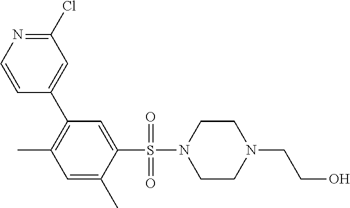 [SMILES: Cc1cc(C)c(S(=O)(=O)N2CCN(CCO)CC2)cc1-c1ccnc(Cl)c1] |
| SR- 35464 | 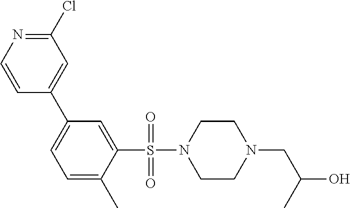 [SMILES: Cc1ccc(-c2ccnc(Cl)c2)cc1S(=O)(=O)N1CCN(CC(C)O)CC1] |
| SR- 35129 | 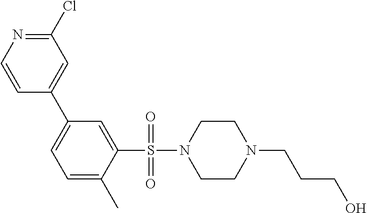 [SMILES: Cc1ccc(-c2ccnc(Cl)c2)cc1S(=O)(=O)N1CCN(CCCO)CC1] |
| SR- 35465 | 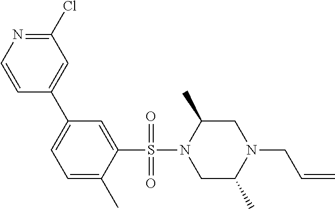 [SMILES: C=CCN1C[C@H](C)N(S(=O)(=O)c2cc(-c3ccnc(Cl)c3)ccc2C)C[C@H]1C] |
| SR- 35324 | 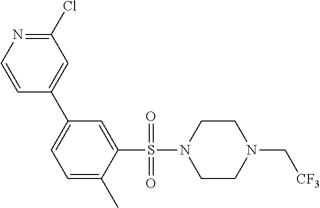 [SMILES: Cc1ccc(-c2ccnc(Cl)c2)cc1S(=O)(=O)N1CCN(CC(F)(F)F)CC1] |
| SR- 34464 | 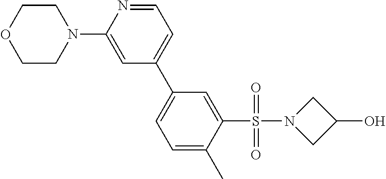 [SMILES: Cc1ccc(-c2ccnc(N3CCOCC3)c2)cc1S(=O)(=O)N1CC(O)C1] |

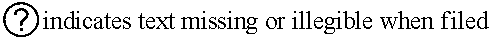

In embodiments, the compound has the structure of Formula (IV),

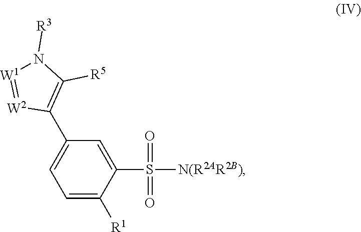

wherein:
W¹ is a —N═ or —CH═;
W² is a —N═ or —CR⁴═

Each R³, R⁴, and R⁵ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹, R^{2A}, and R^{2B} are as described herein.

In embodiments, the compound has the structure of Formula (XIII),

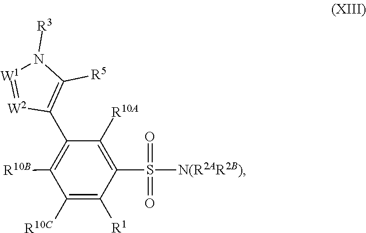

wherein:
provided that when R¹ is hydrogen, then R^{2A} and R^{2B} together with the nitrogen atom form a substituted or unsubstituted heterocycloalkyl, or substituted or unsubstituted heteroaryl. W¹, W², R¹, R^{2A}, R^{2B}, R³, R⁵, R^{10A}, R^{10B}, and R^{10C} are as described herein.

In embodiments, when R¹ is hydrogen, then R^{2A} and R^{2B} together with the nitrogen atom form a substituted or unsubstituted morphorinyl.

In embodiment, R³ is independently hydrogen, halogen, substituted or unsubstituted alkyl, substituted or unsubstituted phenyl. In embodiment, R⁴ is independently hydrogen, substituted or unsubstituted alkyl, substituted or unsubstituted phenyl. In embodiment, R⁵ is independently hydrogen, substituted or unsubstituted alkyl, substituted or unsubstituted phenyl.

In embodiments, in Formula (IV) or (XIII), R^{2A} and R^{2B} together with the nitrogen attached thereto form a

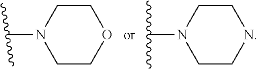

In embodiments, in Formula (IV) or (XIII), R^{2A} and R^{2B} together with the nitrogen attached thereto form a

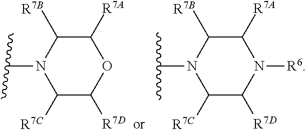

R⁶, R^{7A}, R^{7B}, R^{7C}, and R^{7D} are as described herein.

In embodiments, W¹ is a —N═. In embodiments, W¹ is —CH═. In embodiments, W² is a —N═. In embodiments, W² is —CR⁴═. In embodiments, R⁴ is hydrogen.

In embodiments, the compound has the Formula (IV-a),

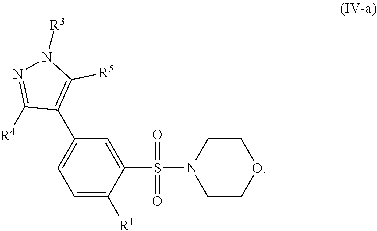

R¹, R³, R⁴, and R⁵ are as described herein.

In embodiments, the compound has the Formula (XIII-a)

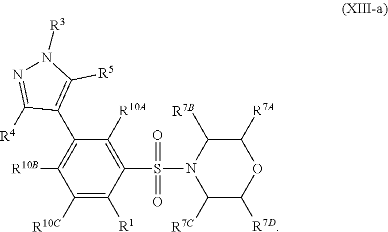

R¹, R³, R^{7A}, R^{7B}, R^{7C}, R^{7D}, R^{10A}, R^{10B}, and R^{10C} are as described herein.

In embodiments, in Formula (IV-a), R⁴ and R⁸ are hydrogen, and R³ is —CH₃,

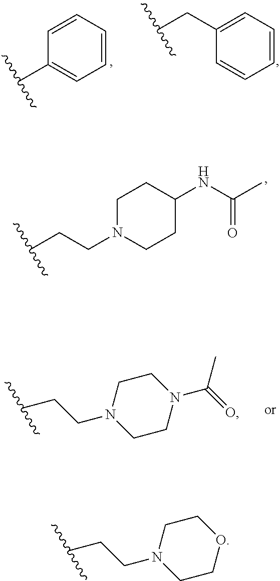 [SMILES: *c1ccccc1 ; *Cc1ccccc1 ; *CCN1CCC(NC(C)=O)CC1 ; *CCN1CCN(C(C)=O)CC1 ; *CCN1CCOCC1]

In embodiments, in Formula (IV-a), R³, R⁴ and R⁸ are hydrogen or —CH₃.

In embodiments, the compound has the Formula (IV-b),

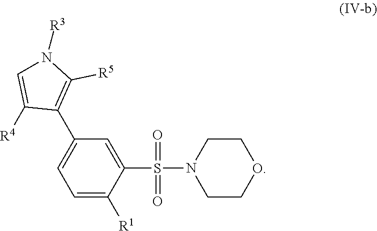

R¹, R³, R⁴, and R⁵ are as described herein.

In embodiments, the compound has the Formula (XIII-b),

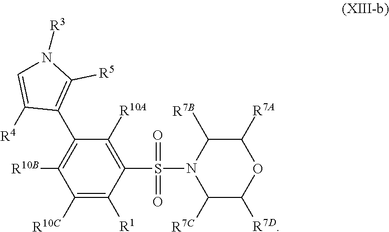

R¹, R³, R^{7A}, R^{7B}, R^{7C}, R^{7D}, R^{10A}, R^{10B}, and R^{10C} are as described herein.

In embodiments, in Formula (IV-b) or (XIII-b), R³ and R⁸ are hydrogen, and R⁴ is hydrogen, and R⁴ is

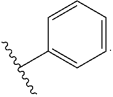 [SMILES: *c1ccccc1]

In embodiments, the compound has the Formula (IV-c),

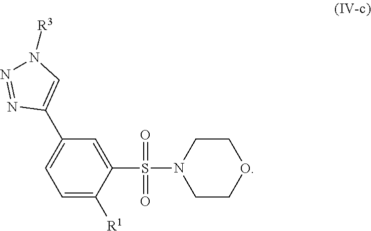

R¹, and R³ are as described herein.

In embodiments, the compound has the Formula (XIII-c),

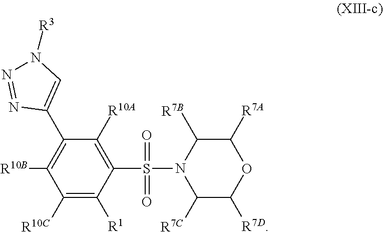

R¹, R³, R^{7A}, R^{7B}, R^{7C}, R^{7D}, R^{10A}, R^{10B}, and R^{10C} are as described herein.

In embodiments, the compound has the Formula (XIII-d), (XIII-e), or (XIII-f),

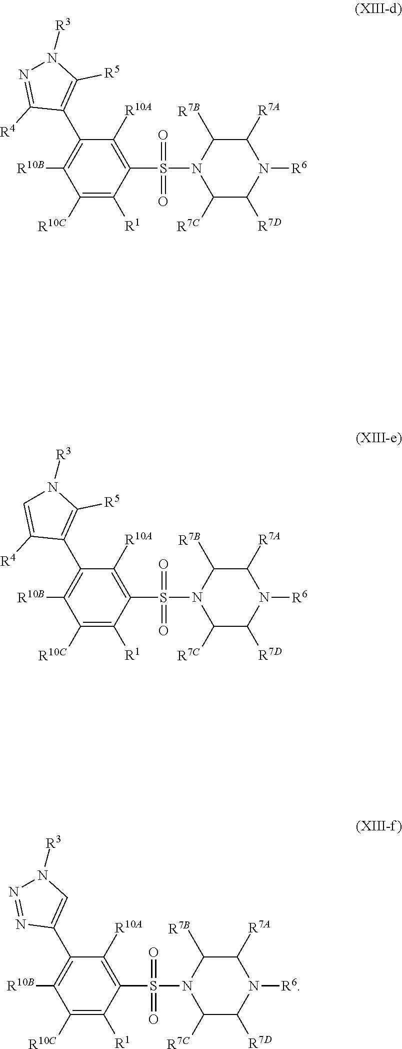

R¹, R³, R^{7A}, R^{7B}, R^{7C}, R^{7D}, R^{10A}, R^{10B}, and R^{10C} are as described herein.

In embodiments, in formula (IV-c) or (XIII-c), R⁴ and R⁵ are hydrogen, and R³ is

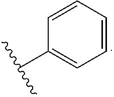 [SMILES: *c1ccccc1]

In embodiments, in formula (XIII-d), R⁴ and R⁵ are hydrogen, and R³ is

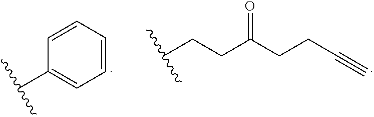

In embodiments, R⁴ and R⁵ are hydrogen, and R³ is —CH₃,

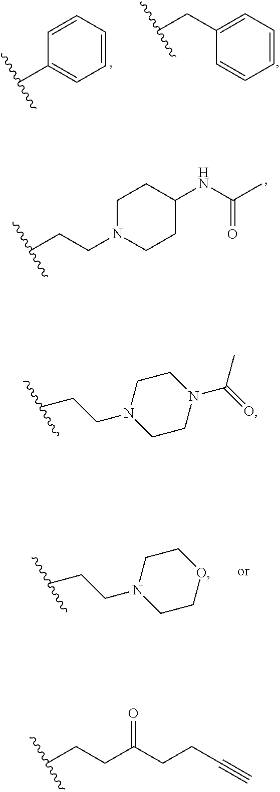

In embodiments, R³, R⁴ and R⁵ are hydrogen. In embodiments, R³ is independently hydrogen, halogen, substituted or unsubstituted alkyl, or substituted or unsubstituted phenyl. In embodiments, R³ is —CH₃.

In embodiments, R⁶ is —H,

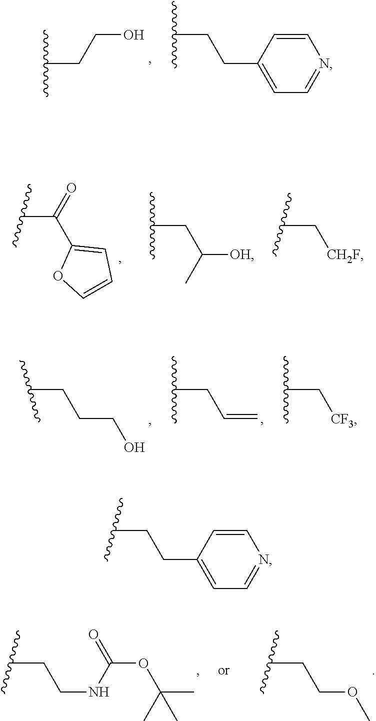

In embodiments, R¹ is —CH₃.

In embodiments, each R^{10A}, R^{10B} and R^{10C} is independently hydrogen, halogen, or —CH₃. In embodiments, R^{10A} is hydrogen, halogen, or —CH₃. In embodiments, R^{10B} is hydrogen, halogen, or —CH₃. In embodiments, R^{10C} is hydrogen, halogen, or —CH₃.

In embodiments, R^{10A} is hydrogen. In embodiments, R^{10B} is hydrogen, —F, or —CH₃. In embodiments, R^{10C} is hydrogen, —F, or —CH₃.

Exemplary compound of Formula (IV) or (XIII) are shown in Table 3.

TABLE 3
Compound of Formula (IV) or (XIII)

| Compound | Structure |
|---|---|
| SR-25864 | 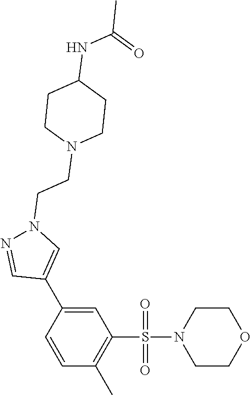 [SMILES: CC(=O)NC1CCN(CCn2cc(-c3ccc(C)c(S(=O)(=O)N4CCOCC4)c3)cn2)CC1] |
| SR-28925 | 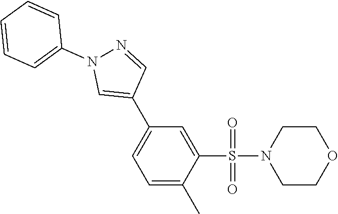 [SMILES: Cc1ccc(-c2cnn(-c3ccccc3)c2)cc1S(=O)(=O)N1CCOCC1] |
| SR-24581 | 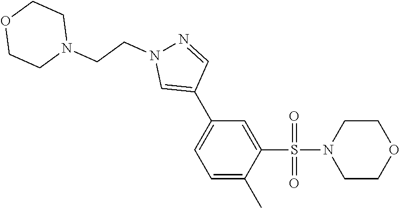 [SMILES: Cc1ccc(-c2cnn(CCN3CCOCC3)c2)cc1S(=O)(=O)N1CCOCC1] |
| SR-29084 | 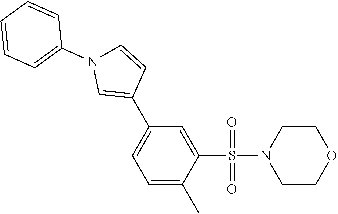 [SMILES: Cc1ccc(-c2ccn(-c3ccccc3)c2)cc1S(=O)(=O)N1CCOCC1] |
| SR-26484 | 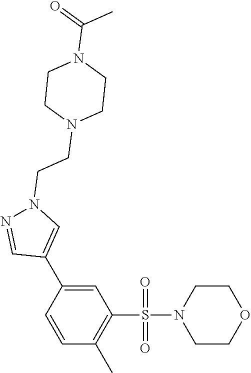 [SMILES: CC(=O)N1CCN(CCn2cc(-c3ccc(C)c(S(=O)(=O)N4CCOCC4)c3)cn2)CC1] |
| SR-28764 | 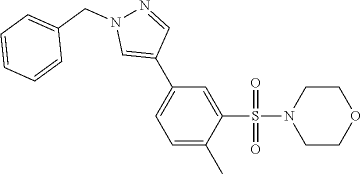 [SMILES: Cc1ccc(-c2cnn(Cc3ccccc3)c2)cc1S(=O)(=O)N1CCOCC1] |
| SR-24578 | 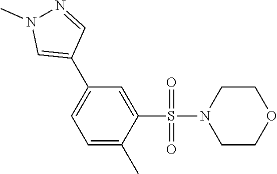 [SMILES: Cc1ccc(-c2cnn(C)c2)cc1S(=O)(=O)N1CCOCC1] |
| SR-29285 | 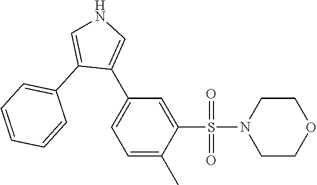 [SMILES: Cc1ccc(-c2c[nH]cc2-c2ccccc2)cc1S(=O)(=O)N1CCOCC1] |
| SR-30084 | 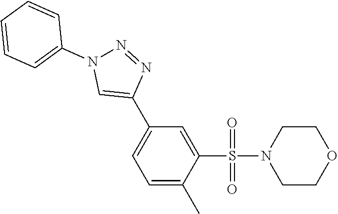 [SMILES: Cc1ccc(-c2cn(-c3ccccc3)nn2)cc1S(=O)(=O)N1CCOCC1] |
| SR-35186 | 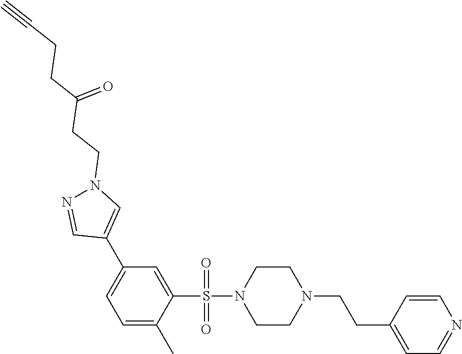 [SMILES: C#CCCC(=O)CCn1cc(-c2ccc(C)c(S(=O)(=O)N3CCN(CCc4ccncc4)CC3)c2)cn1] |
| SR-25584 | 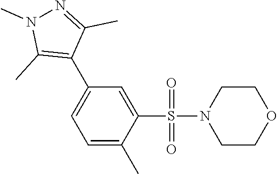 [SMILES: Cc1ccc(-c2c(C)nn(C)c2C)cc1S(=O)(=O)N1CCOCC1] |

In embodiments, the compound has the structure of Formula (V) or (VI),

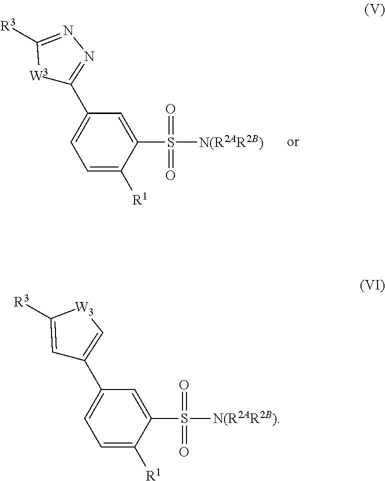

wherein:
W³ is a —S— or —O—;
R³ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R¹, R^{2A}, and R^{2B} are as described herein.

In embodiments, the compound has the structure of Formula (XIV) or (XV),

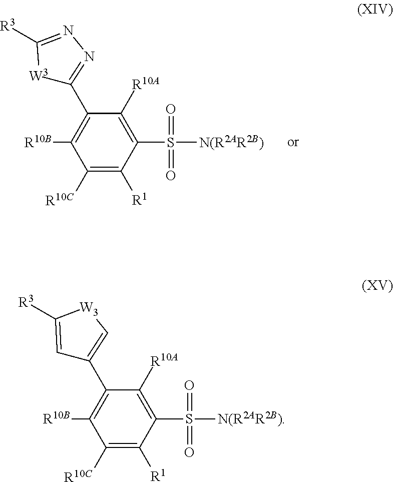

W³, R¹, R³, R^{2A}, R^{2B}, R^{10A}, R^{10B}, and R^{10C} are as described herein.

In embodiments, R³ is hydrogen. In embodiments, R³ is a substituted or unsubstituted C₁-C₄ alkyl. In embodiments, R³ is substituted or unsubstituted phenyl.

In embodiments, R^{2A} and R^{2B} together with the nitrogen attached thereto form a

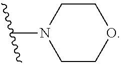 [SMILES: *N1CCOCC1]

In embodiments, R^{2A} and R^{2B} together with the nitrogen attached thereto form

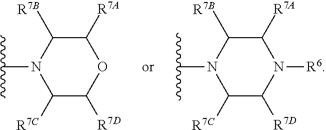

R⁶, R^{7A}, R^{7B}, R^{7C}, and R^{7D} are as described herein.

In embodiments, the compound has the structure of Formula (V-a),

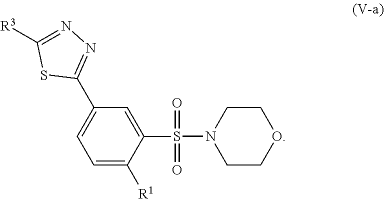

R¹ and R³ are as described herein.

In embodiments, the compound has the structure of Formula (V-b),

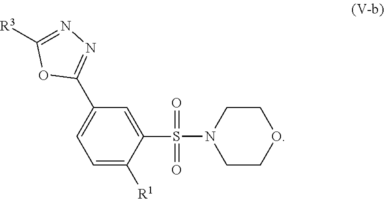

R¹ and R³ are as described herein.

In embodiments, the compound has the structure of Formula (VI-a),

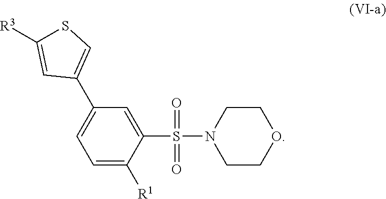

R¹ and R³ are as described herein.

In embodiments, the compound has the structure of Formula (VI-b),

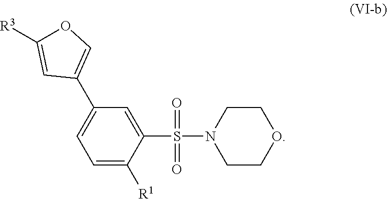

R¹ and R³ are as described herein.

In embodiments, the compound has the structure of Formula (XIV-a),

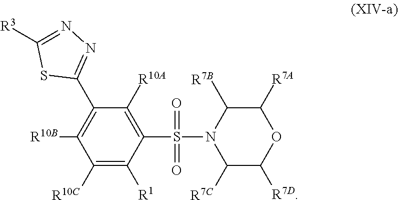

R¹, R³, R^{7A}, R^{7B}, R^{7C}, R^{7D}, R^{10A}, R^{10B}, and R^{10C} are as described herein.

In embodiments, the compound has the structure of Formula (XIV-b),

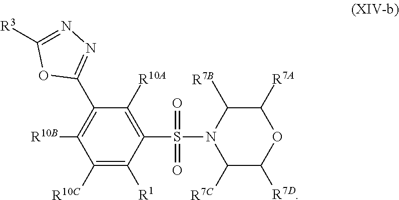

R¹, R³, R^{7A}, R^{7B}, R^{7C}, R^{7D}, R^{10A}, R^{10B} and R^{10C} are as described herein.

In embodiments, the compound has the structure of Formula (XV-a),

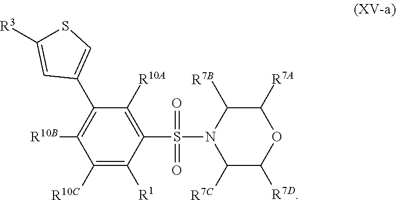

R¹, R³, R^{7A}, R^{7B}, R^{7C}, R^{7D}, R^{10A}, R^{10B} and R^{10C} are as described herein.

In embodiments, the compound has the structure of Formula (XV-b),

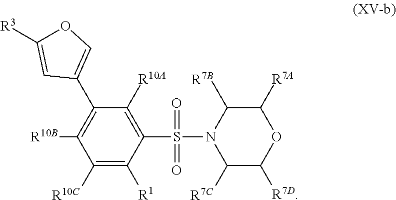

R¹, R³, R^{7A}, R^{7B}, R^{7C}, R^{7D}, R^{10A}, R^{10B} and R^{10C} are as described herein.

In embodiments, R³ is

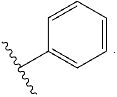 [SMILES: *c1ccccc1]

In embodiments, R¹ is —CH₃.

In embodiments, each R^{10A}, R^{10B} and R^{10C} is independently hydrogen, halogen, or —CH₃. In embodiments, R^{10A} is hydrogen, halogen, or —CH₃. In embodiments, R^{10B} is hydrogen, halogen, or —CH₃. In embodiments, R^{10C} is hydrogen, halogen, or —CH₃.

Exemplary compounds of Formulae (V), (VI), (XIV) and (XV) are shown in Table 4.

TABLE 4
Compound of Formula (V), (VI), (XIV) and (XV)

| Compound | Structure |
|---|---|
| SR-27558 | 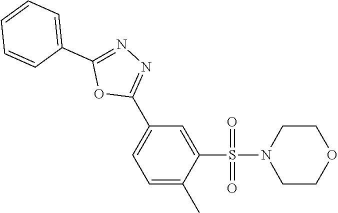 [SMILES: Cc1ccc(-c2nnc(-c3ccccc3)o2)cc1S(=O)(=O)N1CCOCC1] |
| SR-27524 | 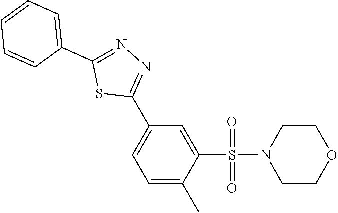 [SMILES: Cc1ccc(-c2nnc(-c3ccccc3)s2)cc1S(=O)(=O)N1CCOCC1] |
| SR-29044 | 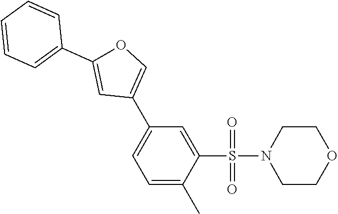 [SMILES: Cc1ccc(-c2coc(-c3ccccc3)c2)cc1S(=O)(=O)N1CCOCC1] |
| SR-29184 | 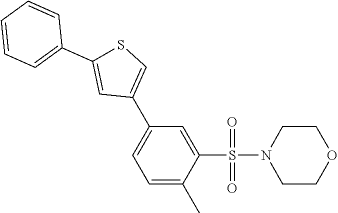 [SMILES: Cc1ccc(-c2csc(-c3ccccc3)c2)cc1S(=O)(=O)N1CCOCC1] |

In embodiments, the Ring A is bi-cyclic heteroaryl. In embodiments, the Ring A is selected from

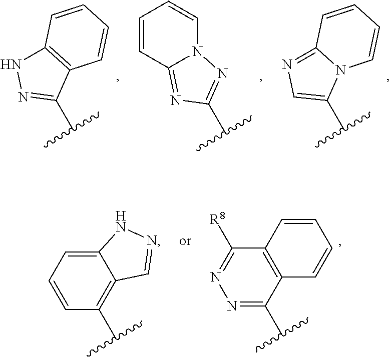

wherein R⁸ is hydrogen, or substituted or unsubstituted alkyl; the Ring A is unsubstituted or substituted with one or more R³, and R³ is independently halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

In embodiments, R⁸ is hydrogen. In embodiments, R⁸ is substituted or unsubstituted C₁₋₃ alkyl. In embodiments, R⁸ is unsubstituted C₁₋₃ alkyl. In embodiments, R⁸ is —CH₃. In embodiments, R⁸ is —CF₃. In embodiments, R⁸ is substituted or unsubstituted C₁₋₃ alkyl.

In embodiments, when Ring A is unsubstituted

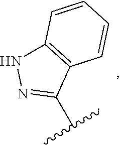 [SMILES: *c1n[nH]c2ccccc12]

—N(R^{2A}R^{2B}) is not a substituted piperidinyl. In embodiments, when Ring A is unsubstituted

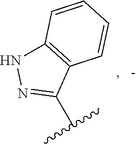 [SMILES: *c1n[nH]c2ccccc12]

N(R^{2A}R^{2B}) is not a 4-substituted piperidinyl.

In embodiments, R^{2A} and R^{2B} together with the nitrogen attached thereto form

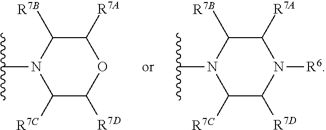

R⁶, R^{7A}, R^{7B}, R^{7C}, and R^{7D} are as described herein.

In embodiments, R⁶, R^{7A}, R^{7B}, R^{7C}, and R^{7D} are hydrogen.

In embodiments, R^{2A} and R^{2B} together with the nitrogen attached thereto form a

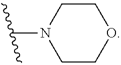 [SMILES: *N1CCOCC1]

In embodiments, R^{2A} and R^{2B} together with the nitrogen attached thereto form

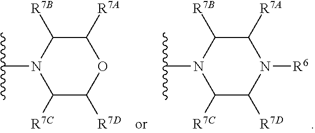

R⁶, R^{7A}, R^{7B}, R^{7C}, and R^{7D} are as described herein.

In embodiments, R³ is

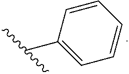 [SMILES: *c1ccccc1]

In embodiments, R¹ is —CH₃.

In embodiments, each R^{10A}, R^{10B} and R^{10C} is independently hydrogen, halogen, or —CH₃. In embodiments, R^{10A} is hydrogen, halogen, or —CH₃. In embodiments, R^{10B} is hydrogen, halogen, or —CH₃. In embodiments, R^{10C} is hydrogen, halogen, or —CH₃.

Exemplary compounds having Ring A of bicyclic heteroaryl are shown in Table 5.

TABLE 5
Compound having bicyclic-heteroaryl Rign A.

| Compound | Structure |
|---|---|
| SR-27564 | 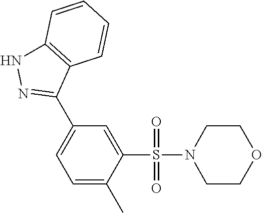 [SMILES: Cc1ccc(-c2n[nH]c3ccccc23)cc1S(=O)(=O)N1CCOCC1] |
| SR-26524 | 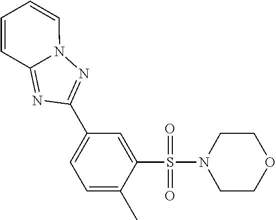 [SMILES: Cc1ccc(-c2nc3ccccn3n2)cc1S(=O)(=O)N1CCOCC1] |
| SR-27444 | 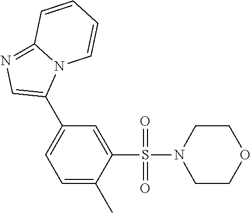 [SMILES: Cc1ccc(-c2cnc3ccccn23)cc1S(=O)(=O)N1CCOCC1] |
| SR-27484 | 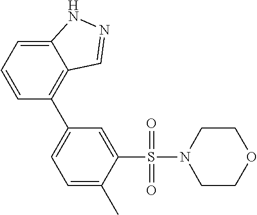 [SMILES: Cc1ccc(-c2cccc3[nH]ncc23)cc1S(=O)(=O)N1CCOCC1] |
| SR-26424 | 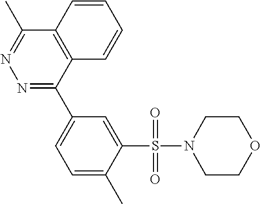 [SMILES: Cc1ccc(-c2nnc(C)c3ccccc23)cc1S(=O)(=O)N1CCOCC1] |

### Pharmaceutical Compositions

In an aspect, provided is a pharmaceutical composition including the compound described herein, a pharmaceutically acceptable salt form thereof, an isomer thereof, or a crystal form thereof. Also provided herein are pharmaceutical formulations. In embodiments, the pharmaceutical formulation includes a compound (e.g. formulae (I), (II), (III), (IV), (V), (X), (XI), (XII), (XIII), (XIV), (XV) and subordinate formulae thereof) including all embodiments thereof, or compounds in Tables 1-5 described above) and a pharmaceutically acceptable excipient.

The pharmaceutical composition may contain a dosage of the compound in a therapeutically effective amount.

In embodiments, the pharmaceutical composition includes any compound described above.

1. Formulations

The pharmaceutical composition may be prepared and administered in a wide variety of dosage formulations. Compounds described may be administered orally, rectally, or by injection (e.g. intravenously, intramuscularly, intracutaneously, subcutaneously, intraduodenally, or intraperitoneally).

For preparing pharmaceutical compositions from compounds described herein, pharmaceutically acceptable carriers can be either solid or liquid. Solid form preparations include powders, tablets, pills, capsules, cachets, suppositories, and dispersible granules. A solid carrier may be one or more substance that may also act as diluents, flavoring agents, binders, preservatives, tablet disintegrating agents, or an encapsulating material.

In powders, the carrier may be a finely divided solid in a mixture with the finely divided active component. In tablets, the active component may be mixed with the carrier having the necessary binding properties in suitable proportions and compacted in the shape and size desired.

The powders and tablets preferably contain from 5% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight. Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The pharmaceutical compositions may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The pharmaceutical composition may be intended for intravenous use. The pharmaceutically acceptable excipient can include buffers to adjust the pH to a desirable range for intravenous use. Many buffers including salts of inorganic acids such as phosphate, borate, and sulfate are known.

2. Effective Dosages

The pharmaceutical composition may include compositions wherein the active ingredient is contained in a therapeutically effective amount, i.e., in an amount effective to achieve its intended purpose. The actual amount effective for a particular application will depend, inter alia, on the condition being treated.

The dosage and frequency (single or multiple doses) of compounds administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds disclosed herein.

Therapeutically effective amounts for use in humans may be determined from animal models. For example, a dose for humans can be formulated to achieve a concentration that has been found to be effective in animals. The dosage in humans can be adjusted by monitoring response of the constipation or dry eye to the treatment and adjusting the dosage upwards or downwards, as described above.

Dosages may be varied depending upon the requirements of the subject and the compound being employed. The dose administered to a subject, in the context of the pharmaceutical compositions presented herein, should be sufficient to effect a beneficial therapeutic response in the subject over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered compounds effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

3. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the compound is used.

When parenteral application is needed or desired, particularly suitable admixtures for the compounds included in the pharmaceutical composition may be injectable, sterile solutions, oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. Pharmaceutical admixtures suitable for use in the pharmaceutical compositions presented herein may include those described, for example, in Pharmaceutical Sciences (17th Ed., Mack Pub. Co., Easton, PA) and WO 96/05309, the teachings of both of which are hereby incorporated by reference.

### Methods

In an aspect, provided is a method for inhibiting NAD consumption and/or increasing NAD synthesis in a patient, and the method includes administering to the patient an effective dose of a compound (e.g. formulae (I), (II), (III), (IV), (V), (X), (XI), (XII), (XIII), (XIV), (XV) and subordinate formulae thereof) including all embodiments thereof, or compounds in Tables 1-5) described above.

The compound can inhibit NAD consuming reactions such as protein ADP-ribosylation reactions. The compound can inhibit NAD cleavage by protein deacetylases or NAD hydrolases. The compound can increase NAD synthesis. The compound can activate enzymes of the NAD synthetic pathways such as the rate-limiting enzyme for NAD synthesis in the salvage pathway called NAMPT. The patient is afflicted with, or at risk for, a protein misfolding neurodegenerative disease or another protein misfolding disease.

The protein misfolding neurodegenerative disease includes a prion disease, Parkinson's disease, dementia with Lewy Bodies, multiple system atrophy or other synucleinopathies, Alzheimer's disease, amyotrophic lateral sclerosis, fronto-temporal dementia or other tauopathy, chronic traumatic encephalopathy, and the protein misfolding disease includes diabetes mellitus and amyloidoses.

In an aspect, provided is a method for preventing or inhibiting NAD depletion in a patient. In another aspect, provided is a method for increasing NAD levels to improve cellular function. In another aspect, provided is a method for improving a condition linked to alterations of NAD metabolism in a patient. The method includes administering to the patient an effective dose of the compound described herein.

The condition includes a metabolic disorder, a liver disorder, aging, a degenerative disease, a neurodegenerative disease, neuronal degeneration associated with multiple sclerosis, hearing loss, multiple sclerosis, retinal damage, macular degeneration, brain or cardiac ischemia, kidney failure, kidney disease, traumatic brain injury, or an axonopathy.

In an aspect, provided is a method for providing protection from toxicity of misfolded proteins in a patient. The method includes administering to the patient an effective dose of the compound described herein. The patient is afflicted with a prion disease, Parkinson's disease or other synucleinopathy, Alzheimer's disease, amyotrophic lateral sclerosis, a tauopathy, an amyloidosis or diabetes mellitus.

In an aspect, provided is a method for preventing or treating a protein misfolding neurodegenerative disease in a patient. The method includes administering to the patient an effective dose of the compound described herein.

In embodiments, the protein misfolding neurodegenerative disease is a disorder associated with protein aggregate-induced neurodegeneration and NAD depletion. In embodiments, the protein misfolding neurodegenerative disease includes a prion disease, Parkinson's disease, dementia with Lewy Bodies, multiple system atrophy or other synucleinopathy, Alzheimer's disease, amyotrophic lateral sclerosis, fronto-temporal dementia or other tauopathy, chronic traumatic encephalopathy. In embodiments, the neurodegenerative disease is multiple sclerosis, brain ischemia or an axonopathy.

In embodiments, the metabolic disorder includes diabetes or a liver disorder.

In embodiments, the condition linked to alterations of NAD metabolism includes aging, a retinal disease or a kidney disease.

In an aspect, provided is a method of preventing or treating a retinal disease in a patient. The method includes administering to the patient an effective dose of the compound described herein.

In an aspect, provided is a method of preventing or treating diabetes, non alcoholic fatty liver disease or other metabolic disease in a patient, comprising administering to the patient an effective dose of the compound described herein.

In an aspect, provided is a method of preventing or treating a kidney disease in a patient, comprising administering to the patient an effective dose of the compound described herein.

In an aspect, provided is a method of mitigating health effects of aging, comprising administering to the patient an effective dose of the compound described herein.

### EXAMPLES

### Examples 1-94: Chemical Synthesis Procedures

General Protocols. Materials were purchased from commercial vendors and used without purification. All moisture-sensitive reactions were performed under argon. Experiments were monitored by LCMS or TLC and visualized using an ultraviolet lamp (254 nm) or staining with KMnO₄. Purification via silica gel flash column chromatography was performed using a Teledyne ISCO Combiflash® Rf+ and Luknova silica gel cartridges. Purification via preparatory HPLC was performed on either an Agilent 1260 Infinity II series or a Shimadzu LC-8A instrument each using a Prep-C18 column (250×30 mm) with a flow rate of 30 mL/min, UV detection at 254, 280, and/or 210 nm, and reverse phase solvent system (A=0.1% TFA in de-ionized water and B=1:1 ACN/MeOH). All NMR data was collected at room temperature on a Bruker Ultrashield 400 MHz nuclear magnetic resonance spectrometer. Chemical shifts for ¹H NMR spectra are reported in parts per million (ppm) relative to residual solvent signal as an internal standard: DMSO (δ 2.50), CHCl₃ (δ 7.26), or MeOH (δ 3.31). Multiplicities are given as: s (singlet), d (doublet), t (triplet), q (quartet), or m (multiplet). Coupling constants are reported as a J value in Hertz (Hz). Mass spectra were recorded on a Thermo Scientific 3000 LCQ Fleet system (ESI) using a Discovery® HS C18 HPLC column (10 cm×2.1 mm, 5 μm) at 35° C. with UV detection at 254 nm. Flow rate was 0.7 mL/min using a solvent gradient of 5-95% B over 4 min (total run time=6 min), where A=0.1% formic acid in de-ionized water and B=0.1% formic acid in ACN. All compounds were dissolved in 100% DMSO as 10 mM stocks.

Abbreviations. Certain abbreviations for common chemicals were used in the Examples and are defined as follows:
ACN═acetonitrile
AgNO₃=silver nitrate
Br₂=bromine
1-BuOH=1-butanol
CDCl₃=deuterated chloroform
CD₃OD=deuterated methanol
(CD₃)₂CO=deuterated acetone
(CD₃)₂SO=deuterated DMSO
CsF=cesium fluoride
Cs₂CO₃=cesium carbonate
CuI=copper iodide
Cu(OAc)₂=copper acetate
DCE=dichloroethane
DCM=dichloromethane
DIPEA=diisopropylethylamine
DME=dimethoxyethane
DMF=N,N-dimethylformamide
DMSO=dimethylsulfoxide
Dppf=1,1′-bis(diphenylphosphino)ferrocene
EA=ethyl acetate
ESI=Electrospray ionization mass spectroscopy
Et₃N═triethylamine
Et₂O=diethylether
EtOH=ethanol
H₂SO₄=sulfuric acid
HATU=1-[Bis(dimethylamino)methylene]-1H-1,2,3-triazolo[4,5-b]pyridinium 3-oxide hexafluorophosphate
HCl=hydrochloric acid
HPLC=high performance liquid chromatography
K₂CO₃=potassium carbonate
KOAc=potassium acetate
LC-MS=liquid chromatography-mass spectroscopy
MeOH=methanol
MeI=methyl iodide
NaH=sodium hydride
NaHCO₃=sodium bicarbonate
Na₂CO₃=sodium carbonate
NaOCl=sodium hyopochlorite
NaN₃=sodium azide
Na₂S=sodium sulfide
Na₂SO₄=sodium sulfate
NBS=N-bromosuccinimide
NH₄OH=ammonium hydroxide
NMR=nuclear magnetic resonance spectroscopy
Pd₂(dba)₃=tris(dibenzylideneacetone)palladium (0)
Pd(PPh₃)₄=tetrakis(triphenylphosphine)palladium (0)
Pd(dppf)Cl₂·CH₂Cl₂=[1,1′-Bis(diphenylphosphino)ferrocene]dichloropalladium(II)
complex with DCM
PdCl₂(PPh₃)₂=Dichlorobis(triphenylphosphine)palladium(II)
SOCl₂=thionyl chloride
TBAF=tetrabutylammonium fluoride
TFA=trifluoroacetic acid
THE=tetrahydrofuran
TIPS=triisopropylsilyl
Zn(CN)₂=zinc cyanide
ZnBr₂=zinc bromide

Reaction Schemes. Compounds of the invention are synthesized as shown in the following general reaction schemes, representative schematic examples, and experimental details as provided in the Examples.

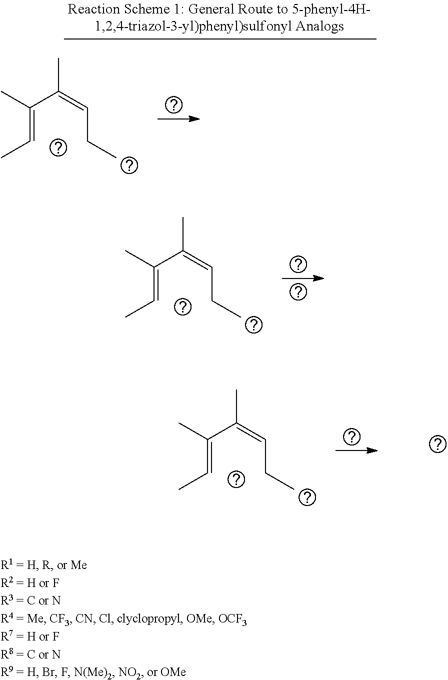

### Representative Example 1: Synthesis of SR-25604

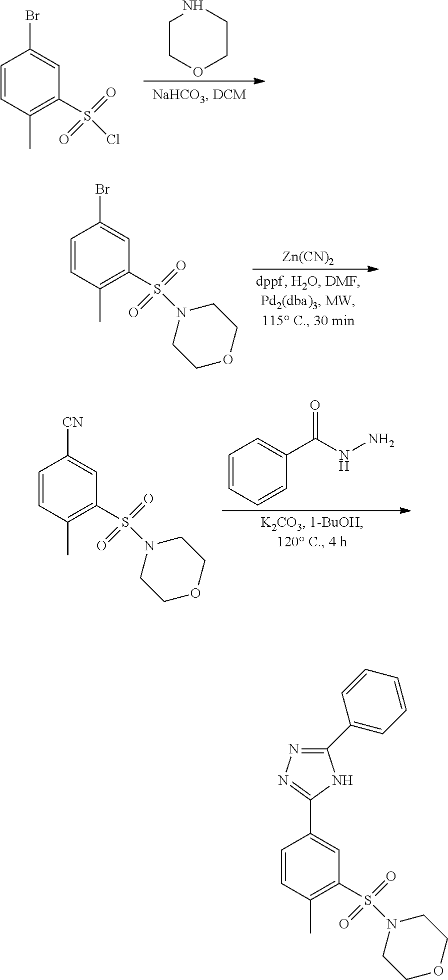

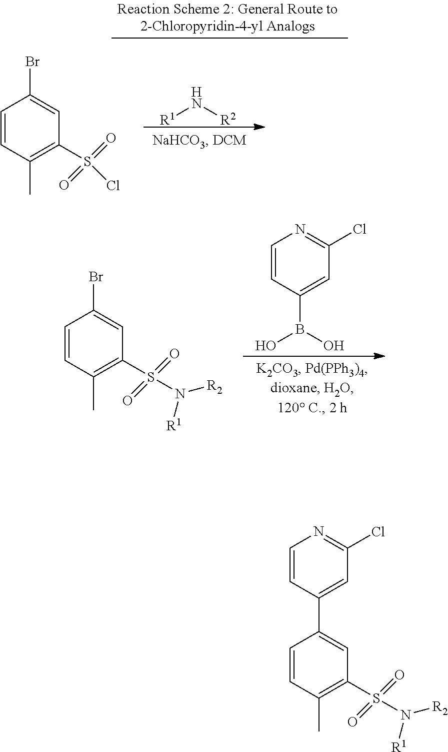

### Representative Example 2: Synthesis of SR-34793

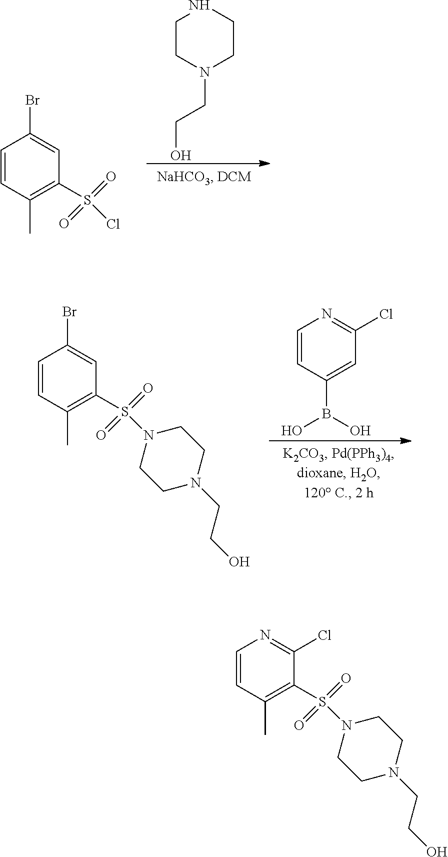 [SMILES: Cc1ccc(Br)cc1S(=O)(=O)Cl ; OCCN1CCNCC1 ; Cc1ccc(Br)cc1S(=O)(=O)N1CCN(CCO)CC1 ; OB(O)c1ccnc(Cl)c1 ; Cc1ccnc(Cl)c1S(=O)(=O)N1CCN(CCO)CC1]

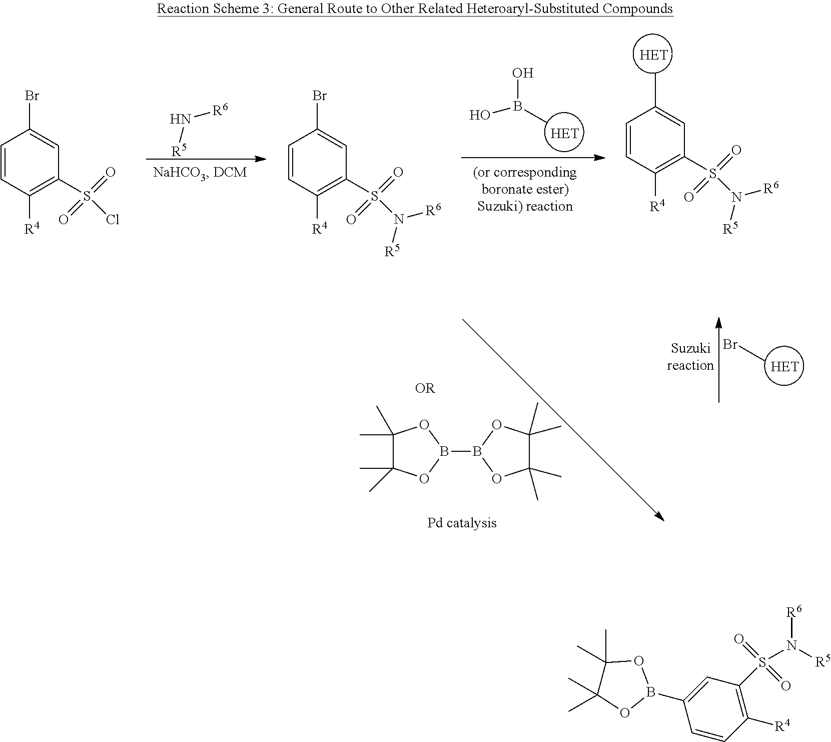

### Representative Example 3. Synthesis of 4-((2-Methyl-5-(1H-pyrazol-4-yl)phenyl)sulfonyl)morpholine

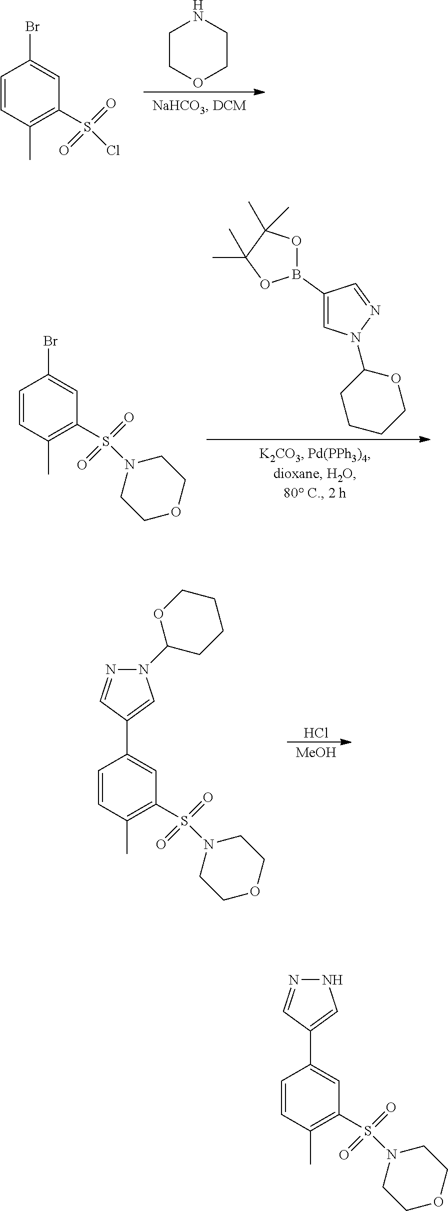

### Example 1. 4-((5-Bromo-2-methylphenyl)sulfonyl)morpholine

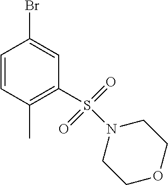 [SMILES: Cc1ccc(Br)cc1S(=O)(=O)N1CCOCC1]

A mixture of 5-bromo-2-methylbenzene sulfonyl chloride (320 mg, 1.2 mmol), NaHCO₃ (1 g, 12 mmol), and morpholine (123 μL, 1.4 mmol) in DCM was stirred overnight at room temperature. Upon completion, the solvent was removed under reduced pressure and purification via column chromatography afforded product 4-((5-bromo-2-methylphenyl)sulfonyl)morpholine (380 mg, 100%).

### Example 2. 4-((5-(2-Chloropyridin-4-yl)-2-methylphenyl)sulfonyl)morpholine

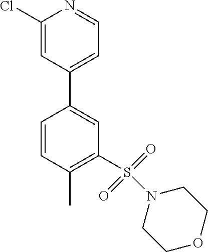 [SMILES: Cc1ccc(-c2ccnc(Cl)c2)cc1S(=O)(=O)N1CCOCC1]

A mixture of 4-((5-bromo-2-methylphenyl)sulfonyl)morpholine (50 mg, 0.16 mmol), K₂CO₃ (65 mg, 0.47 mmol), de-ionized water (400 μL), and 2-chloro-pyridine-4-boronic acid (29 mg, 0.18 mmol) in 1,4-dioxane (1.6 mL) was de-gassed and back-filled with argon 3 times at room temperature in a microwave vial. Pd(PPh₃)₄ (9 mg, 0.008 mmol) was added, the vial was sealed, and the mixture was de-gassed and back-filled with argon. Then, the reaction mixture was microwaved in a Biotage© microwave reactor at 120° C. for 2 hours under normal absorption conditions. Upon completion, solvent was removed under reduced pressure and purification via column chromatography afforded pure product 4-((5-(2-Chloropyridin-4-yl)-2-methylphenyl)sulfonyl)morpholine (45 mg, 82% yield). ¹H NMR (CDCl₃) δ 8.45 (d, J=5.2 Hz, 1H), 8.14 (d, J=2.0 Hz, 1H), 7.73 (dd, J=7.6, 2.0 Hz, 1H), 7.54 (t, J=0.6 Hz, 1H), 7.48 (d, J=8.0 Hz, 1H), 3.73 (t, J=4.8 Hz, 4H), 3.18 (t, J=4.8 Hz, 4H), 2.69 (s, 3H); MS (m/z): [M] calc'd for C₁₆H₁₇ClN₂O₃S is 352.06. found 352.96 [M+H].

### Example 3. N-(2-((4-(4-methyl-3-(morpholinosulfonyl)phenyl)pyridin-2-yl)amino)ethyl) acetamide (SR-25104)

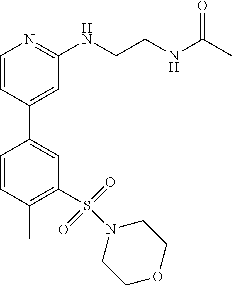 [SMILES: CC(=O)NCCNc1cc(-c2ccc(C)c(S(=O)(=O)N3CCOCC3)c2)ccn1]

A mixture of 4-((5-(2-Chloropyridin-4-yl)-2-methylphenyl)sulfonyl)morpholine (23 mg, 0.06 mmol), CsF (50 mg, 0.3 mmol), and ethylenediamine (250 μL, 3.7 mmol) in 1,4-dioxane (250 μL) was stirred at 120° C. for 40 h and then was cooled to room temperature. Upon completion, solvent and excess ethylenediamine were removed under reduced pressure to afford N-(4-(4-methyl-3-(morpholinosulfonyl)phenyl)pyridin-2-yl)ethane-1,2-diamine (30 mg). MS (m/z): [M] calc'd for C₁₈H₂₄N₄O₃S is 376.16. found 376.95.

Then, a mixture of the crude product (30 mg) and NaHCO₃ (67 mg, 0.8 mmol) in DCM was cooled in an ice-water bath. Acetyl chloride (5.1 μL, 0.07 mmol) in DCM was added dropwise and the reaction mixture was stirred for 2 hours in the ice-water bath. Upon completion, solvent was removed under reduced pressure and purification via prep HPLC followed by column chromatography afforded pure product SR-25104 (16 mg, 48% yield). ¹H NMR (CD₃OD) δ 8.16 (d, J=2.0 Hz, 1H), 7.59 (d, J=6.0 Hz, 1H), 7.91 (dd, J=8.0, 2.0 Hz, 1H), 7.59 (d, J=8.0 Hz, 1H), 7.06-7.03 (m, 2H), 3.70 (t, J=4.6 Hz, 4H), 3.53 (t, J=6.0 Hz, 2H), 3.43 (t, J=6.0 Hz, 2H), 3.16 (t, J=4.8 Hz, 4H), 2.70 (s, 3H), 1.96 (s, 3H); MS (m/z): [M] calc'd for C₂₀H₂₆N₄O₄S is 418.17 [M]. found 418.95 [M+H].

### Example 4. 4-Methyl-3-(morpholinosulfonyl)-N-(pyridin-4-ylmethyl)aniline (SR-25464)

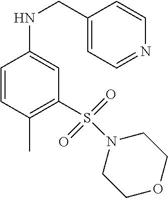 [SMILES: Cc1ccc(NCc2ccncc2)cc1S(=O)(=O)N1CCOCC1]

A mixture of 4-((5-bromo-2-methylphenyl)sulfonyl)morpholine (25 mg, 0.08 mmol), Cs₂CO₃ (51 mg, 0.16 mmol), xantphos (1.3 mg, 0.002 mmol), and 4-(aminomethyl)pyridine (16 μL, 0.16 mmol) in 1,4-dioxane (800 μL) was de-gassed and back-filled with argon 3 times at room temperature in a microwave vial. Pd₂(dba)₃ (1.8 mg, 0.002 mmol) was added, the vial was sealed, and the mixture was de-gassed and back-filled with argon. Then, the reaction mixture was microwaved in a Biotage© microwave reactor at 90° C. for 2 hours under normal absorption conditions. Upon completion, solvent was removed under reduced pressure and purification via prep HPLC afforded pure product SR-25464 (17 mg, 63% yield). ¹H NMR (CD₃OD) δ 8.77, (s, 1H), 8.06 (d, J=5.6 Hz, 2H), 7.15 (d, J=8.4 Hz, 1H), 7.02 (d, J=2.4 Hz, 1H), 6.77 (dd, J=8.2, 2.6 Hz, 1H), 4.72 (s, 2H), 3.65 (t, J=4.8 Hz, 4H), 3.00 (t, J=4.8 Hz, 4H), 2.45 (s, 3H); MS (m/z): [M] calc'd for C₁₇H₂₁N₃O₃S is 347.13. found 348.01 [M+H].

### Example 5. N-methyl-N-(2-(methyl(4-(4-methyl-3-(morpholinosulfonyl)phenyl)pyridin-2-yl)amino)ethyl)acetamide (SR-25124)

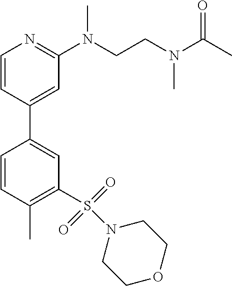 [SMILES: CC(=O)N(C)CCN(C)c1cc(-c2ccc(C)c(S(=O)(=O)N3CCOCC3)c2)ccn1]

A mixture of 4-((5-(2-Chloropyridin-4-yl)-2-methylphenyl)sulfonyl)morpholine (30 mg, 0.09 mmol), CsF (65 mg, 0.43 mmol), and N,N′-dimethylethylenediamine (55 μL, 5.1 mmol) was microwaved in a Biotage© microwave reactor at 120° C. for 13 hours under high absorption conditions. Upon completion, excess N,N′-dimethylethylenediamine was removed under reduced pressure to afford N¹,N²-dimethyl-N¹-(4-(4-methyl-3-(morpholinosulfonyl)phenyl)pyridin-2-yl)ethane-1,2-diamine (33 mg). MS (m/z): [M] calc'd for C₂₀H₂₈N₄O₃S is 404.19. found 404.93 [M+H].

Then, a mixture of the crude product (33 mg) and NaHCO₃ (71 mg, 0.8 mmol) in DCM was cooled in an ice-water bath. Acetyl chloride (4.8 μL, 0.07 mmol) in DCM was added dropwise and the reaction mixture was stirred for 2 hours in the ice-water bath. Upon completion, solvent was removed under reduced pressure and purification via column chromatography afforded pure product SR-25124 (28 mg, 77% yield). ¹H NMR (CD₃OD) δ 8.61 (d, J=5.6 Hz, 1H), 8.23 (d, J=2.0 Hz, 1H), 7.99 (dd, J=8.0, 2.0 Hz, 1H), 7.86 (d, J=1.2 Hz, 1H), 7.73 (dd, J=5.2, 1.2 Hz, 1H), 7.62 (d, J=8.0 Hz, 1H), 4.06 (t, J=6.8 Hz, 2H), 3.70 (t, J=4.8 Hz, 4H), 3.17 (t, J=4.8 Hz, 4H), 2.74 (t, J=6.8 Hz, 2H), 2.71 (s, 3H), 2.42 (s, 6H), 2.04 (s, 3H); MS (m/z): [M] calc'd for C₂₂H₃₀N₄O₄S is 446.20. found 446.97 [M+H].

### Example 6. 4-((2-Methyl-5-(1,3,5-trimethyl-1H-pyrazol-4-yl)phenyl)sulfonyl)morpholine (SR-25584)

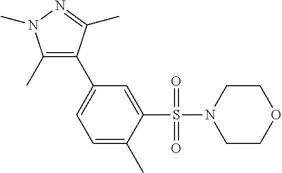 [SMILES: Cc1ccc(-c2c(C)nn(C)c2C)cc1S(=O)(=O)N1CCOCC1]

A mixture of 4-((5-bromo-2-methylphenyl)sulfonyl)morpholine (25 mg, 0.08 mmol), K₂CO₃ (54 mg, 0.39 mmol), and 1,3,5-trimethyl-1H-pyrazole-4-boronic acid pinacol ester (20 mg, 0.14 mmol) in DME/de-ionized water (1:1, 1.4 mL) was de-gassed and back-filled with argon 3 times at room temperature in a microwave vial. Pd(PPh₃)₄ (9 mg, 0.008 mmol) was added, the vial was sealed, and the mixture was de-gassed and back-filled with argon. Then, the reaction mixture was microwaved in a Biotage® microwave reactor at 100° C. for 5 min under normal absorption conditions. Upon completion, solvent was removed under reduced pressure and purification via prep HPLC afforded pure product SR-25584 (24 mg, 88% yield). ¹H NMR (CD₃OD) δ 7.58 (d, J=1.2 Hz, 1H), 7.52-7.46 (m, 2H), 3.84 (s, 3H), 3.69 (t, J=4.6 Hz, 4H), 3.13 (t, J=4.8 Hz, 4H), 2.66 (s, 3H), 2.30 (s, 3H), 2.25 (s, 3H); MS (m/z): [M] calc'd for C₁₇H₂₃N₃O₃S is 349.15. found 349.91.

### Example 7. 4-((2-Methyl-5-(1H-pyrazol-4-yl)phenyl)sulfonyl)morpholine

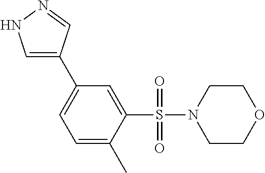 [SMILES: Cc1ccc(-c2cn[nH]c2)cc1S(=O)(=O)N1CCOCC1]

A mixture of 4-((5-bromo-2-methylphenyl)sulfonyl)morpholine (41 mg, 0.13 mmol), K₂CO₃ (35 mg, 0.25 mmol), and 1-(tetrahydro-2H-pyran-2-yl)-4-(4,4,5,5-tetramethyl-1,3,2-dioxaborolan-2-yl)-1H-pyrazole (43 mg, 0.15 mmol) in 1,4-dioxane/de-ionized water (4:1, 1.3 mL) was de-gassed and back-filled with argon 3 times at room temperature in a microwave vial. Pd(PPh₃)₄ (7.4 mg, 0.006 mmol) was added, the vial was sealed, and the mixture was de-gassed and back-filled with argon. Then, the reaction mixture was stirred at 80° C. in a pre-heated oil bath for 2 hours. Upon completion, solvent was removed under reduced pressure and purification via column chromatography afforded product 4-((2-methyl-5-(1-(tetrahydro-2H-pyran-2-yl)-1H-pyrazol-4-yl)phenyl)sulfonyl)morpholine (82 mg). MS (m/z): [M] calc'd for C₁₉H₂₅N₃O₄S is 391.16. found 391.43 [M+H].

Then, a mixture of the product (82 mg) and HCl (41 μL, 0.5 mmol) in MeOH was heated to 50° C. overnight. Upon completion, solvent was removed under reduced pressure and purification via prep HPLC afforded pure product 4-((2-methyl-5-(1H-pyrazol-4-yl)phenyl)sulfonyl)morpholine (35 mg, 89% yield over 2 steps). MS (m/z): [M] calc'd for C₁₄H₁₇N₃O₃S is 307.10. found 307.77 [M+H].

### Example 8. 4-((2-Methyl-5-(1-(methylsulfonyl)-1H-pyrazol-4-yl)phenyl)sulfonyl)morpholine

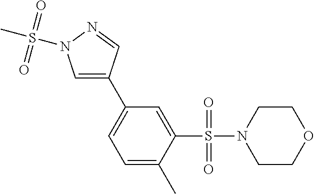 [SMILES: Cc1ccc(-c2cnn(S(C)(=O)=O)c2)cc1S(=O)(=O)N1CCOCC1]

A mixture of 4-((2-methyl-5-(1H-pyrazol-4-yl)phenyl)sulfonyl)morpholine (60 mg, 0.2 mmol), pyridine (46 μL, 0.57 mmol), and methanesulfonyl chloride (30 μL, 0.39 mmol) in DCM was stirred overnight at room temperature. Upon completion, solvent and excess pyridine were removed under reduced pressure and purification via column chromatography afforded pure product 4-((2-methyl-5-(1-(methylsulfonyl)-1H-pyrazol-4-yl)phenyl)sulfonyl)morpholine (70 mg, 88% yield). MS (m/z): [M] calc'd for C₁₅H₁₉N₃O₅S₂ is 385.08. found 385.78 [M+H].

### Example 9. 1-(4-(2-(4-(4-Methyl-3-(morpholinosulfonyl)phenyl)-1H-pyrazol-1-yl)ethyl)piperazin-1-yl)ethan-1-one (SR-25484)

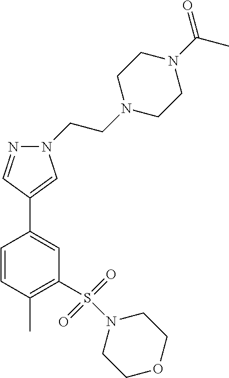 [SMILES: CC(=O)N1CCN(CCn2cc(-c3ccc(C)c(S(=O)(=O)N4CCOCC4)c3)cn2)CC1]

A mixture of 4-((2-methyl-5-(1-(methylsulfonyl)-1H-pyrazol-4-yl)phenyl)sulfonyl)morpholine (60 mg, 0.16 mmol), tert-butyl-4-(2-hydroxyethyl)piperazine-1-carboxylate (30 mg, 0.13 mmol), and sodium tert-butoxide (15 mg, 0.16 mmol) in DMF was stirred at 90° C. overnight. Upon completion, solvent was removed under reduced pressure and purification via column chromatography afforded product tert-butyl 4-(2-(4-(4-methyl-3-(morpholinosulfonyl)phenyl)-1H-pyrazol-1-yl)ethyl)piperazine-1-carboxylate (50 mg). MS (m/z): [M] calc'd for C₂₅H₃₇N₅O₅S is 519.25. found 519.78 [M+H].

Then, a mixture of the product (50 mg) and TFA/DCM (1:1) was stirred overnight at room temperature. Upon completion, solvent was removed under reduced pressure to yield the crude product 4-((2-methyl-5-(1-(2-(piperazin-1-yl)ethyl)-1H-pyrazol-4-yl)phenyl)sulfonyl)morpholine as a TFA salt (50 mg). MS (m/z): [M] calc'd for C₂₀H₂₉N₅O₃S is 419.20. found 419.99 [M+H].

Lastly, a mixture of the crude product (50 mg), NaHCO₃ (162 mg, 1.9 mmol), and acetyl chloride (13.8 μL, 0.19 mmol) in DCM was stirred overnight at room temperature. Upon completion, solvent was removed under reduced pressure and purification via column chromatography afforded pure product SR-25484 (14 mg, 19% yield over 3 steps). ¹H NMR (CD₃OD) δ 8.04 (s, 1H), 7.89 (d, J=1.6 Hz, 1H), 7.78 (s, 1H), 7.63 (dd, J=8.0, 1.6 Hz, 1H, 7.31 (d, J=8.0 Hz, 1H), 4.24 (t, J=6.2 Hz, 2H), 3.59 (t, J=4.6 Hz, 4H), 3.46-3.41 (m, 4H), 3.03 (t, J=4.6 Hz, 4H), 2.80 (t, J=6.0 Hz, 2H), 2.51 (s, 3H), 2.47-2.41 (m, 4H), 1.97 (s, 3H); MS (m/z): [M] calc'd for C₂₂H₃₁N₅O₄S is 461.21. found 462.03 [M+H].

### Example 10. 4-((2-Methyl-5-(5-phenyl-4H-1,2,4-triazol-3-yl)phenyl)sulfonyl)morpholine (SR-25604)

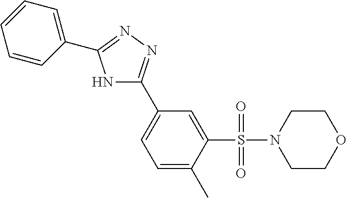 [SMILES: Cc1ccc(-c2nnc(-c3ccccc3)[nH]2)cc1S(=O)(=O)N1CCOCC1]

A mixture of 4-((5-bromo-2-methylphenyl)sulfonyl)morpholine (50 mg, 0.16 mmol), dppf (4.8 mg, 0.009 mmol), de-ionized water (31 μL), and Zn(CN)₂ (18 mg, 0.15 mmol) in DMF (3.1 mL) was de-gassed and back-filled with argon 3 times at room temperature in a microwave vial. Pd₂(dba)₃ (3.1 mg, 0.003 mmol) was added, the vial was sealed, and the mixture was de-gassed and back-filled with argon. Then, the reaction mixture was microwaved in a Biotage© microwave reactor at 115° C. for 30 min under normal absorption conditions. Upon completion, solvent was removed under reduced pressure and purification via column chromatography afforded pure product 4-methyl-3-(morpholinosulfonyl)benzonitrile (35 mg, 84% yield).

Then, a mixture of the product (35 mg, 0.13 mmol), K₂CO₃ (37 mg, 0.27 mmol), and benzhydrazide (18 mg, 0.13 mmol) in 1-BuOH (131 μL) was stirred at 120° C. in a pre-heated oil bath for 4 hours. Upon completion, solvent was removed under reduced pressure and purification via column chromatography followed by prep HPLC afforded pure product SR-25604 (16 mg, 24% yield). ¹H NMR (CD₃OD) δ 8.61 (d, J=1.6 Hz, 1H), 8.25 (dd, J=8.0, 2.0 Hz, 1H), 8.06 (dd, J=7.8, 1.8 Hz, 2H), 7.59-7.53 (m, 4H), 3.72 (t, J=4.6 Hz, 4H), 3.20 (t, J=4.8 Hz, 4H), 2.71 (s, 3H); MS (m/z): [M] calc'd for C₁₉H₂₀N₄O₃S is 384.13, found 384.96 [M+H].

### Example 11. N-(1-(2-(4-(4-methyl-3-(morpholinosulfonyl)phenyl)-1H-pyrazol-1-yl)ethyl)piperidin-4-yl)acetamide (SR-25864)

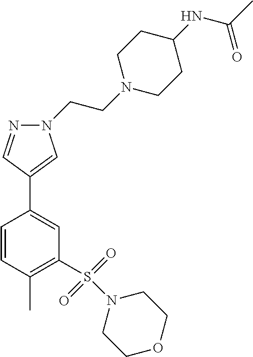 [SMILES: CC(=O)NC1CCN(CCn2cc(-c3ccc(C)c(S(=O)(=O)N4CCOCC4)c3)cn2)CC1]

A mixture of 4-boc-aminopiperidine (500 mg, 2.5 mmol), 2-bromoethanol (375 mg, 3.0 mmol), and K₂CO₃ (1.1 g, 8 mmol) in ACN was stirred overnight at 50° C. Upon completion, solvent was removed under reduced pressure and purification via column chromatography afforded product tert-butyl (1-(2-hydroxyethyl)piperidin-4-yl)carbamate.

Then, a mixture of the product (63 mg, 0.26 mmol) with 4-((2-methyl-5-(1-(methylsulfonyl)-1H-pyrazol-4-yl)phenyl)sulfonyl)morpholine (70 mg, 0.18 mmol) and sodium tert-butoxide (20 mg, 0.21 mmol) in DMF (0.61 mL) was stirred overnight at 90° C. in a pre-heated oil bath. Upon completion, solvent was removed under reduced pressure and purification via column chromatography afforded pure product tert-butyl (1-(2-(4-(4-methyl-3-(morpholinosulfonyl)phenyl)-1H-pyrazol-1-yl)ethyl)piperidin-4-yl)carbamate (70 mg, 72% yield). MS (m/z): [M] calc'd for C₂₆H₃₉N₅O₅S is 533.27. found 533.97 [M+H].

Then, a mixture of the product (70 mg) and TFA/DCM (1:1) was stirred overnight at room temperature. Upon completion, solvent was removed under reduced pressure to yield the crude product 1-(2-(4-(4-methyl-3-(morpholinosulfonyl)phenyl)-1H-pyrazol-1-yl)ethyl)piperidin-4-amine as a TFA salt (72 mg). MS (m/z): [M] calc'd for C₂₁H₃₁N₅O₃S is 433.21. found 434.06 [M+H].

Lastly, a mixture of the crude product (72 mg), NaHCO₃ (880 mg, 10.5 mmol), and acetyl chloride (76 μL, 1.1 mmol) in DCM was stirred overnight at room temperature. Upon completion, solvent was removed under reduced pressure and purification via column chromatography afforded pure product SR-25864 (28 mg, 45% yield over 2 steps). ¹H NMR (CD₃OD) δ 8.13 (s, 1H), 7.99 (d, J=2.0 Hz, 1H), 7.89, d, J=0.4 Hz, 1H), 7.73 (dd, J=7.6, 2.0 Hz, 1H), 7.41 (d, J=8.0 Hz, 1H), 4.39 (t, J=6.6 Hz, 2H), 3.69 (t, J=4.8 Hz, 5H), 3.13 (t, J=4.8 Hz, 4H), 3.05 (q, J=6.4 Hz, 4H), 2.61 (s, 3H), 2.45 (t, J=10.8 Hz, 2H), 1.96-1.89 (m, 6H), 1.58 (qd, J=5.6, 3.4 Hz, 2H); MS (m/z): [M] calc'd for C₂₃H₃₃N₅O₄S is 475.23. found 475.97 [M+H].

### Example 12. 4-((2-Chloro-4-(5-phenyl-4H-1,2,4-triazol-3-yl)phenyl)sulfonyl)morpholine (SR-26324)

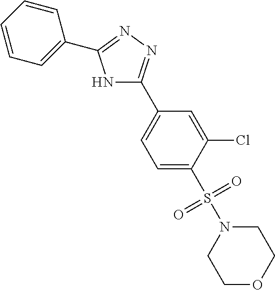 [SMILES: O=S(=O)(c1ccc(-c2nnc(-c3ccccc3)[nH]2)cc1Cl)N1CCOCC1]

This compound was prepared according to the procedure for SR-25604 in 35% overall yield starting from 4-bromo-2-chlorobenzene sulfonyl chloride. ¹H NMR ((CD₃)₂SO) δ 8.30 (s, 1H), 8.23 (dd, J=8.4, 1.6 Hz, 1H), 8.12-8.08 (m, 3H), 7.58-7.56 (m, 3H), 3.63 (t, J=4.6 Hz, 4H), 3.20 (t, J=4.6 Hz, 4H); MS (m/z): [M] calc'd for C₁₈H₁₇ClN₄O₃S is 404.07. found 404.93 [M+H].

### Example 13. 4-((4-(5-Phenyl-4H-1,2,4-triazol-3-yl)-2-(trifluoromethyl)phenyl)sulfonyl) morpholine (SR-26444)

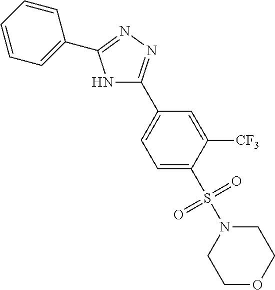 [SMILES: O=S(=O)(c1ccc(-c2nnc(-c3ccccc3)[nH]2)cc1C(F)(F)F)N1CCOCC1]

This compound was prepared according to the procedure for SR-25604 in 31% overall yield starting from 4-bromo-2-(trifluoromethyl)benzene sulfonyl chloride. ¹H NMR (CD₃OD) δ 8.77 (s, 1H), 8.60 (d, J=8.0 Hz, 1H), 8.29 (d, J=8.4 Hz, 1H), 8.12-8.11 (m, 2H), 7.61 (d, J=5.6 Hz, 3H), 3.77 (t, J=4.4 Hz, 4H), 3.31 (t, J=4.4 Hz, 4H); MS (m/z): [M] calc'd for C₁₉H₁₇F₃N₄O₃S is 438.10. found 438.95 [M+H].

### Example 14. 1-Bromo-4-methyl-phthalazine

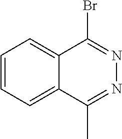

A mixture of 2-acetylbenzoic acid (500 mg, 3.0 mmol), K₂CO₃ (700 mg, 5.1 mmol), MeI (700 μL, 11.2 mmol) in DMF stirred overnight at room temperature. Upon completion, solvent was removed under reduced pressure and purification via column chromatography afforded pure product methyl 2-acetylbenzoate (526 mg, 97% yield). MS (m/z): [M] calc'd for C₁₀H₁₀O₃ is 178.06. found 178.58 [M+H].

Then, a mixture of the product (526 mg, 3.0 mmol) and hydrazine monohydrate (207 μL, 4.4 mmol) in EtOH stirred overnight at room temperature. Upon completion, solvent was removed under reduced pressure and purification via column chromatography afforded pure product 4-methylphthalazin-1(2H)-one (163 mg, 34% yield). MS (m/z): [M] calc'd for C₉H₈N₂O is 160.06. found 160.77 [M+H].

Lastly, a mixture of the product (163 mg, 1.0 mmol) and POBr₃ (583 mg, 2.0 mmol) was stirred 4 hours at 120° C. in a pre-heated oil bath and then was poured on ice. The mixture was basified with NH₄OH_{(aq)} and the solid precipitate was collected by filtration to 1-bromo-4-methyl-phthalazine (217 mg, 96% yield). MS (m/z): [M] calc'd for C₉H₉BrN₂ is 221.98/223.98. found 222.86/224.86 [M+H].

### Example 15. 4-((2-Methyl-5-(4-methylphthalazin-1-yl)phenyl)sulfonyl)morpholine (SR-26424)

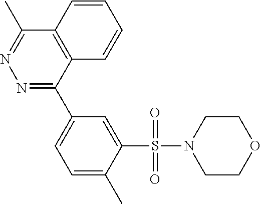

A mixture of 4-((5-bromo-2-methylphenyl)sulfonyl)morpholine (50 mg, 0.16 mmol), KOAc (60 mg, 0.61 mmol), bis(pinacolato)diboron (45 mg, 0.18 mmol) in 1,4-dioxane (1.6 mL) was de-gassed and back-filled with argon 3 times at room temperature in a microwave vial. Pd(dppf)Cl₂·CH₂Cl₂ (12 mg, 0.018 mmol) was added, the vial was sealed, and the mixture was de-gassed and back-filled with argon. Then, the reaction mixture was microwaved in a Biotage© microwave reactor at 120° C. for 30 min under normal absorption conditions. Upon completion, solvent was removed under reduced pressure and purification via column chromatography afforded pure product 4-((2-methyl-5-(4,4,5,5-tetramethyl-1,3,2-dioxaborolan-2-yl)phenyl)sulfonyl)morpholine (51 mg, 89% yield). MS (m/z): [M] calc'd for C₁₇H₂₆BNO₅S is 367.16. found 367.86 [M+H].

Then, a mixture of the product (51 mg, 0.14 mmol), Na₂CO_{3(aq)} (208 μL, 2M, 0.42 mmol), and 1-bromo-4-methyl-phthalazine (37 mg, 0.17 mmol) in DME (1.4 mL) was de-gassed and back-filled with argon 3 times at room temperature in a microwave vial. Pd(dppf)Cl₂·CH₂Cl₂ (8.4 mg, 0.011 mmol) was added, the vial was sealed, and the mixture was de-gassed and back-filled with argon. Then, the reaction mixture was microwaved in a Biotage© microwave reactor at 110° C. for 30 min under normal absorption conditions. Upon completion, solvent was removed under reduced pressure and purification via prep HPLC followed by column chromatography afforded pure product SR-26424 (8 mg, 15% yield). ¹H NMR (CD₃OD) δ 8.39 (d, J=8.0 Hz, 1H), 8.19 (d, J=1.6 Hz, 1H), 8.12-8.08 (m, 1H), 8.03-8.01 (m, 2H), 7.90 (dd, J=7.8, 1.8 Hz, 1H), 7.69 (d, J=8.0 Hz, 1H), 3.71 (t, J=4.6 Hz, 4H), 3.20 (t, J=4.6 Hz, 4H), 3.06 (s, 3H), 2.78 (s, 3H); MS (m/z): [M] calc'd for C₂₀H₂₁N₃O₃S is 383.13. found 382.17 [M+H].

### Example 16. 4-((5-([1,2,4]Triazolo[1,5-a]pyridin-2-yl)-2-methylphenyl)sulfonyl) morpholine (SR-26524)

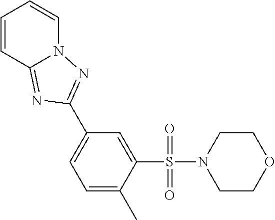 [SMILES: Cc1ccc(-c2nc3ccccn3n2)cc1S(=O)(=O)N1CCOCC1]

A mixture of 4-methyl-3-(morpholinosulfonyl)benzonitrile (51 mg, 0.19 mmol), 2-aminopyridine (22 mg, 0.23 mmol), CuI (1.8 mg, 0.009 mmol), ZnBr₂ (4.3 mg, 0.02 mmol), and 1,10-phenanthroline (1.7 mg, 0.009 mmol) in 1,2-dichlorobenzene (383 μL) was stirred at 130° C. in a pre-heated oil bath for 24 hours. Upon completion, solvent was removed under reduced pressure and purification via column chromatography followed by prep HPLC afforded pure product SR-26524 (4 mg, 5.8% yield). ¹H NMR (CD₃OD) δ 8.83 (dd, J=6.8, 1.2 Hz, 1H) 8.74 (d, J=1.6 Hz, 1H), 8.38 (dd, J=8.0, 2.0 Hz, 1H), 7.80 (dd, J=8.8, 1.2 Hz, 1H), 7.75-7.71 (m, 1H), 7.61 (d, J=8.0 Hz, 1H), 7.26-7.22 (m, 1H), 3.72 (t, J=4.8 Hz, 4H), 3.20 (t, J=4.6 Hz, 4H), 2.72 (s, 3H); MS (m/z): [M] calc'd for C₁₇H₁₈N₄O₃S is 358.11. found 358.98 [M+H].

### Example 17. 2-(Morpholinosulfonyl)-4-(5-phenyl-4H-1,2,4-triazol-3-yl)benzonitrile (SR-27504)

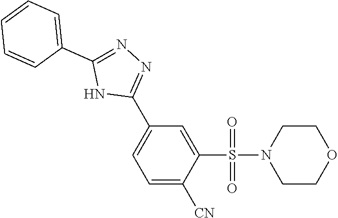 [SMILES: N#Cc1ccc(-c2nnc(-c3ccccc3)[nH]2)cc1S(=O)(=O)N1CCOCC1]

This compound was prepared according to the procedure for SR-25604 in 25% overall yield starting from 5-bromo-2-chlorobenzene sulfonyl chloride. ¹H NMR (CD₃OD) δ 8.76 (s, 1H), 8.55 (d, J=7.6 Hz, 1H), 8.14 (d, J=8.0 Hz, 1H), 8.06-8.05 (m, 2H), 7.57-7.56 (m, 3H), 3.75 (t, J=4.6 Hz, 4H), 3.26 (t, J=4.4H, 4H); MS (m/z): [M] calc'd for C₁₉H₁₇N₅O₃S is 395.11. found 395.91 [M+H].

### Example 18. 4-((2-Methoxy-5-(5-phenyl-4H-1,2,4-triazol-3-yl)phenyl)sulfonyl) morpholine (SR-26624)

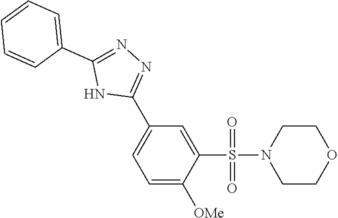 [SMILES: COc1ccc(-c2nnc(-c3ccccc3)[nH]2)cc1S(=O)(=O)N1CCOCC1]

This compound was prepared according to the procedure for SR-25604 in 18% overall yield starting from 5-bromo-2-methoxybenzene sulfonyl chloride. ¹H NMR (CD₃OD) δ 8.57 (d, J=2.0 Hz, 1H), 8.34 (dd, J=8.6, 2.2 Hz, 2H), 8.06 (dd, J=7.8, 1.8 Hz, 2H), 7.54-7.52 (m, 3H), 7.40 (d, J=8.4 Hz, 1H), 4.03 (s, 3H), 3.70 (t, J=4.6 Hz, 4H), 3.25 (t, J=4.8 Hz, 4H); MS (m/z): [M] calc'd for C₁₉H₂₀N₄O₄S is 400.12. found 400.93 [M+H].

### Example 19. 4-((2-Chloro-5-(5-phenyl-4H-1,2,4-triazol-3-yl)phenyl)sulfonyl)morpholine (SR-26525)

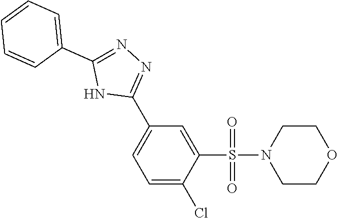 [SMILES: O=S(=O)(c1cc(-c2nnc(-c3ccccc3)[nH]2)ccc1Cl)N1CCOCC1]

This compound was prepared according to the procedure for SR-25604 in 11% overall yield starting from 5-bromo-2-chlorobenzene sulfonyl chloride and using Pd(PPh₃)₄ for the cyanation rather than Pd₂(dba)₃. ¹H NMR (CD₃OD) δ 8.76 (d, J=2.4 Hz, 1H), 8.33 (dd, J=8.2, 2.2 Hz, 1H), 8.05 (dd, J=7.6, 2.0 Hz, 2H), 7.78 (d, J=8.4 Hz, 1H), 7.58-7.53 (m, 3H), 3.71 (t, J=4.6 Hz, 4H), 3.31 (t, J=4.8 Hz, 4H); MS (m/z): [M] calc'd for C₁₈H₁₇ClN₄O₃S is 404.07. found 405.04 [M+H].

### Example 20. 4-((5-(Imidazo[1,2-a]pyridin-3-yl)-2-methylphenyl)sulfonyl)morpholine (SR-27444)

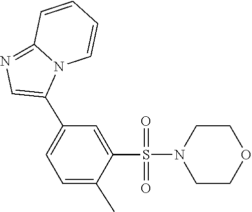 [SMILES: Cc1ccc(-c2cnc3ccccn23)cc1S(=O)(=O)N1CCOCC1]

This compound was prepared according to the procedure for SR-26424 but with 3-bromoimidazo[1,2-a]pyridine in 49% overall yield starting from 4-((5-bromo-2-methylphenyl)sulfonyl)morpholine. ¹H NMR (CD₃OD) δ 8.72 (d, J=6.8 Hz, 1H), 8.27 (s, 1H), 8.20 (d, J=2.0 Hz, 1H), 8.07-8.01 (m, 2H), 7.93 (dd, J=7.6, 2.0 Hz, 1H), 7.73 (d, J=8.0 Hz, 1H), 7.54 (td, J=6.0, 2.2 Hz, 1H), 3.72 (t, J=4.8 Hz, 4H), 3.20 (t, J=4.8 Hz, 4H), 2.77 (s, 3H); MS (m/z): [M] calc'd for C₁₈H₁₉N₃O₃S is 357.11 found 357.96 [M+H].

### Example 21. 4-((5-(1H-Indazol-4-yl)-2-methylphenyl)sulfonyl)morpholine (SR-27484)

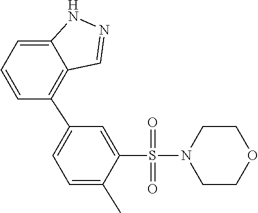 [SMILES: Cc1ccc(-c2cccc3[nH]ncc23)cc1S(=O)(=O)N1CCOCC1]

This compound was prepared according to the procedure for SR-26424 but with tert-butyl-4-(4,4,5,5-tetramethyl-1,3,2-dioxaborolan-2-yl)-1H-indazole-1-carboxylate in 20% overall yield starting from 4-((5-bromo-2-methylphenyl)sulfonyl)morpholine. ¹H NMR (CD₃OD) δ 8.20 (d, J=2.0 Hz, 1H), 8.14 (s, 1H), 7.92 (dd, J=7.8, 1.8 Hz, 1H), 7.59 (d, J=8.0 Hz, 2H), 7.49 (dd, J=8.4, 1.2 Hz, 1H), 7.28 (dd, J=6.8, 0.8 Hz, 1H), 3.71 (t, J=4.8 Hz, 4H), 3.17 (t, J=4.6 Hz, 4H), 2.72 (s, 3H); MS (m/z): [M] calc'd for C₁₈H₁₉N₃O₃S is 357.11 found 357.85 [M+H].

### Example 22. 4-((2-Methyl-5-(5-phenyl-1,3,4-thiadiazol-2-yl)phenyl)sulfonyl)morpholine (SR-27524)

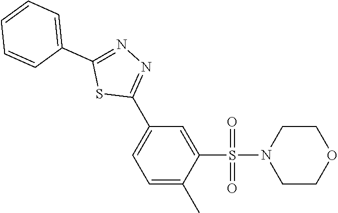 [SMILES: Cc1ccc(-c2nnc(-c3ccccc3)s2)cc1S(=O)(=O)N1CCOCC1]

This compound was prepared according to the procedure for SR-26424 but with 2-bromo-5-phenyl-1,3,4-thiadiazole in 33% overall yield starting from 4-((5-bromo-2-methylphenyl)sulfonyl)morpholine. ¹H NMR (CDCl₃) δ 8.46 (d, J=1.6 Hz, 1H), 8.17 (dd, J=8.0, 2.0 Hz, 1H), 8.03-8.00 (m, 2H), 7.53-7.48 (m, 4H), 3.76 (t, J=4.8 Hz, 4H), 3.24 (t, J=4.8 Hz, 4H), 2.72 (s, 3H); MS (m/z): [M] calc'd for C₁₉H₁₉N₃O₃S₂ is 401.09 found 402.02 [M+H].

### Example 23. 4-((2-Methyl-5-(5-phenyl-1,3,4-oxadiazol-2-yl)phenyl)sulfonyl)morpholine (SR-27558)

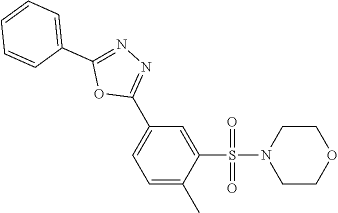 [SMILES: Cc1ccc(-c2nnc(-c3ccccc3)o2)cc1S(=O)(=O)N1CCOCC1]

This compound was prepared according to the procedure for SR-26424 but with 2-bromo-5-phenyl-1,3,4-oxadiazole in 60% overall yield starting from 4-((5-bromo-2-methylphenyl)sulfonyl)morpholine. ¹H NMR (CD₃OD) δ 8.60 (s, 1H), 8.29 (d, J=8.0 Hz, 1H), 8.16 (d, J=7.2 Hz, 2H), 7.68 (d, J=8.4 Hz, 1H), 7.65-7.60 (m, 3H), 3.72 (t, J=4.6 Hz, 4H), 3.21 (t, J=4.4 Hz, 4H), 2.74 (s, 3H); MS (m/z): [M] calc'd for C₁₉H₁₉N₃O₄S is 385.11 found 386.01 [M+H].

### Example 24. 4-((5-(1H-Indazol-3-yl)-2-methylphenyl)sulfonyl)morpholine (SR-27564)

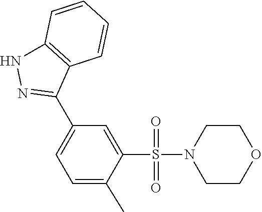 [SMILES: Cc1ccc(-c2n[nH]c3ccccc23)cc1S(=O)(=O)N1CCOCC1]

A mixture of 4-((2-methyl-5-(4,4,5,5-tetramethyl-1,3,2-dioxaborolan-2-yl)phenyl)sulfonyl)morpholine (57 mg, 0.16 mmol), K₂CO₃ (54 mg, 0.39 mmol), and 3-bromoindazole (25 mg, 0.13 mmol) in DME/de-ionized water (1:1, 1.3 mL) was de-gassed and back-filled with argon 3 times at room temperature in a microwave vial. Pd(PPh₃)₄ (7.3 mg, 0.006 mmol) was added, the vial was sealed, and the mixture was de-gassed and back-filled with argon. Then, the reaction mixture was microwaved in a Biotage© microwave reactor at 100° C. for 5 min under normal absorption conditions. Upon completion, solvent was removed under reduced pressure and purification via prep HPLC afforded pure product SR-27564 as a TFA salt (25 mg, 34% yield starting from 4-((5-bromo-2-methylphenyl)sulfonyl)morpholine). ¹H NMR (CD₃OD) δ 8.47 (d, J=1.6 Hz, 1H), 8.16 (dd, J=8.0, 2.0 Hz, 1H), 8.01 (d, J=8.4 Hz, 1H), 7.59 (dd, J=8.2, 2.6 Hz, 2H), 7.45 (td, J=7.6, 0.8 Hz, 1H), 7.26 (td, J=7.6, 0.8 Hz, 1H), 3.72 (t, J=4.6 Hz, 4H), 3.19 (t, J=4.8 Hz, 4H), 2.71 (s, 3H); MS (m/z): [M] calc'd for C₁₈H₁₉N₃O₃S is 357.11 found 357.91 [M+H].

### Example 25. 4-((5-Ethynyl-2-methylphenyl)sulfonyl)morpholine

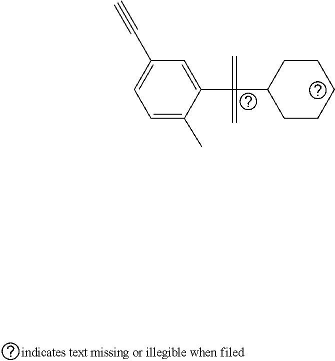

A mixture of 4-((5-bromo-2-methylphenyl)sulfonyl)morpholine (100 mg, 0.31 mmol), trimethylsilylacetylene (56 μL, 0.39 mmol), PdCl₂(PPh₃)₂ (6.5 mg, 0.009 mmol), and CuI (1.7 mg, 0.009 mmol) in 1:1 Et₃N/1,4-dioxane (3 mL) heated overnight at 65° C. Upon completion, solvent was removed under reduced pressure and purification via column chromatography afforded pure product 4-((2-methyl-5-((trimethylsilyl)ethynyl)phenyl)sulfonyl)morpholine (95 mg, 90%). MS (m/z): [M] calc'd for C₁₆H₂₃NO₃SSi is 337.11 found 337.84 [M+H].

Then, a mixture of the product (95 mg, 0.28 mmol) and K₂CO₃ (140 mg, 1.0 mmol) in 3:1 THF/MeOH was stirred overnight at room temperature. Upon completion, the reaction mixture was quenched water and the aqueous layer was extracted with DCM. The combined organic layers were dried over Na₂SO₄ and concentrated to dryness to yield crude 4-((5-ethynyl-2-methylphenyl)sulfonyl)morpholine, which was used without further purification. ¹H NMR (CDCl₃) δ 8.02 (s, 1H), 7.57 (dd, J=8.0, 1.6 Hz, 1H), 7.30 (d, J=8.0 Hz, 1H), 3.73 (t, J=4.8 Hz, 4H), 3.17 (4.6 Hz, 4H), 3.14 (s, 1H), 2.64 (s, 3H).

### Example 26. 4-((2-Methyl-5-(2-phenyl-1H-imidazol-4-yl)phenyl)sulfonyl)morpholine (SR-27824)

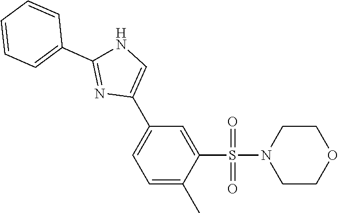 [SMILES: Cc1ccc(-c2c[nH]c(-c3ccccc3)n2)cc1S(=O)(=O)N1CCOCC1]

A mixture of 4-((5-ethynyl-2-methylphenyl)sulfonyl)morpholine (50 mg, 0.19 mmol), NBS (40 mg, 0.22 mmol), and AgNO₃ (3.2 mg, 0.02 mmol) in acetone (630 μL) was stirred for 1 hour at room temperature in an amber vial in the dark. After 1 hour, AgNO₃ (3.2 mg, 0.02 mmol) was added, and the reaction mixture was stirred for an additional 1 hour. Upon completion, the solvent was removed under reduced pressure and purification via column chromatography afforded pure product 4-((5-(bromoethynyl)-2-methylphenyl)sulfonyl)morpholine (51 mg, 79%). MS (m/z): [M] calc'd for C₁₃H₁₄BrNO₃S is 342.99/344.99 found 343.67/345.67.

Then, a mixture of the product (51 mg, 0.15 mmol), benzamidine hydrochloride (35 mg, 0.22 mmol), K₂CO₃ (82 mg, 0.59 mmol), de-ionized water (6.5 μL), and 2,2′-bipyridine (1.2 mg, 0.008 mmol) in toluene (300 μL) was heated to 120° C. for 10 hours. Upon completion, the solvent was removed under reduced pressure and purification via prep HPLC afforded pure product SR-27824 (26 mg, 46%). ¹H NMR (CD₃OD) δ 8.37 (d, J=2.0 Hz, 1H), 8.07 (s, 1H), 8.01 (dt, J=9.6, 2.0 Hz, 3H), 7.71-7.65 (m, 3H), 7.61 (d, J=8.0 Hz, 1H), 3.71 (t, J=4.8 Hz, 4H), 3.19 (t, J=4.8 Hz, 4H), 2.71 (s, 3H); MS (m/z): [M] calc'd for C₂₀H₂₁N₃O₃S is 383.13 found 384.04 [M+H].

### Example 27. 4-((5-(5-(4-Fluorophenyl)-4H-1,2,4-triazol-3-yl)-2-methylphenyl)sulfonyl) morpholine (SR-27964)

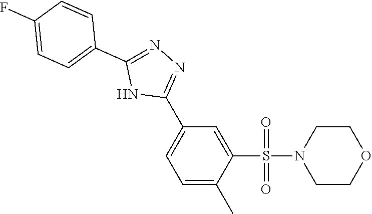 [SMILES: Cc1ccc(-c2nnc(-c3ccc(F)cc3)[nH]2)cc1S(=O)(=O)N1CCOCC1]

This compound was prepared according to the procedure for SR-25604 with 4-fluorobenzhydrazide in 21% overall yield. ¹H NMR (CD₃OD) δ 8.60 (s, 1H), 8.24 (d, J=7.6 Hz, 1H), 8.12-8.09 (m, 2H), 7.59 (d, J=7.6 Hz, 1H), 7.28 (t, J=8.4 Hz, 2H), 3.72 (t, J=4.6 Hz, 4H), 3.19 (t, J=4.6 Hz, 4H), 2.71 (s, 3H); MS (m/z): [M] calc'd for C₁₉H₁₉FN₄O₃S is 402.12. found 402.97 [M+H].

### Example 28. 4-((4-(5-Phenyl-4H-1,2,4-triazol-3-yl)pyridin-2-yl)sulfonyl)morpholine (SR-28004)

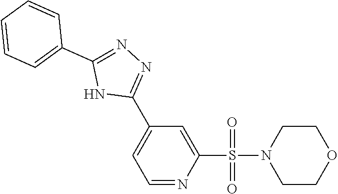 [SMILES: O=S(=O)(c1cc(-c2nnc(-c3ccccc3)[nH]2)ccn1)N1CCOCC1]

A mixture of 2-chloro-4-cyanopyridine (200 mg, 1.4 mmol) and thiourea (110 mg, 1.4 mmol) was refluxed for 4 hours in isopropanol. Upon completion, the solid precipitate was collected by filtration and then was suspended in concentrated H₂SO₄ (4.8 mL). The mixture was cooled in an ice-water bath and NaOCl (18 mL, 14.5%) was added dropwise. The reaction mixture was stirred for 15 min in the ice-water bath and then was diluted with DCM. The aqueous layer was extracted with DCM. The combined organic layers were dried over Na₂SO₄ and concentrated to dryness to yield crude 4-cyanopyridine-2-sulfonyl chloride, which was used without further purification.

Then, a mixture of the crude product (329 mg, 1.6 mmol), NaHCO₃ (1.4 g, 16.7 mmol), and morpholine (168 μL, 1.9 mmol) in DCM was stirred overnight at room temperature. Upon completion, the solvent was removed under reduced pressure and purification via column chromatography afforded pure product 2-(morpholinosulfonyl)isonicotinonitrile (190 mg, 52% yield over 3 steps). ¹H NMR (CDCl₃) δ 8.89 (dd, J=4.8, 0.8 Hz, 1H), 8.12 (t, J=1.0 Hz, 1H), 7.74 (dd, J=5.0, 1.4 Hz, 1H), 3.72 (t, J=4.6 Hz, 4H), 3.32 (t, J=4.8 Hz, 4H).

Lastly, a mixture of the product (30 mg, 0.12 mmol), K₂CO₃ (16 mg, 0.12 mmol), and benzhydrazide (32 mg, 0.24 mmol) in 1-BuOH (118 μL) was stirred for 4 hours at 120° C. in a pre-heated oil bath. Upon completion, the solvent was removed under reduced pressure and purification via prep HPLC afforded pure product SR-28004 (29 mg, 66% yield). ¹H NMR (CD₃OD) δ 8.83 (d, J=4.8 Hz, 1H), 8.60 (s, 1H), 8.30 (d, J=4.8 Hz, 1H), 8.06-8.04 (m, 2H), 7.55 (d, J=5.2 Hz, 3H), 3.72 (t, J=4.6 Hz, 4H), 3.31 (t, J=4.6 Hz, 4H); MS (m/z): [M] calc'd for C₁₇H₁₇N₅O₃S is 371.11. found 371.90 [M+H].

### Example 29. 4-((5-(5-(3,4-Difluorophenyl)-4H-1,2,4-triazol-3-yl)-2-methylphenyl)sulfonyl) morpholine (SR-27984)

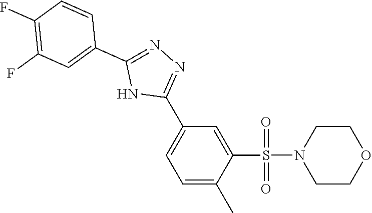 [SMILES: Cc1ccc(-c2nnc(-c3ccc(F)c(F)c3)[nH]2)cc1S(=O)(=O)N1CCOCC1]

This compound was prepared according to the procedure for SR-25604 with 3,4-difluorobenzohydrazide in 42% overall yield. ¹H NMR (CD₃OD) δ 8.58 (d, J=0.8 Hz, 1H), 8.21 (d, J=5.2 Hz, 1H), 8.00-7.96 (m, 1H), 7.91 (dd, J=4.2, 1.0 Hz, 1H), 7.58 (d, J=5.2 Hz, 1H), 7.43 (q, J=6.0 Hz, 1H), 3.72 (t, J=3.0 Hz, 4H), 3.12 (t, J=3.2 Hz, 4H), 2.70 (s, 3H); MS (m/z): [M] calc'd for C₁₉H₁₈F₂N₄O₃S is 420.11. found 420.94 [M+H].

### Example 30. 4-((5-(5-Phenyl-4H-1,2,4-triazol-3-yl)-2-(trifluoromethyl)phenyl)sulfonyl) morpholine (SR-28125)

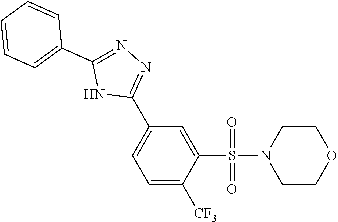 [SMILES: O=S(=O)(c1cc(-c2nnc(-c3ccccc3)[nH]2)ccc1C(F)(F)F)N1CCOCC1]

A mixture of 3-chloro-4-(trifluoromethyl)benzonitrile (100 mg, 0.49 mmol) and Na₂S (76 mg, 0.97 mmol) in DMF (490 μL) was microwaved in a Biotage© microwave reactor at 120° C. for 4 hours under high absorption conditions. Upon completion, solvent was removed under reduced pressure and the residue was suspended in concentrated H₂SO₄ (1.2 mL). The mixture was cooled in an ice-water bath and NaOCl (4.5 mL, 14.5%) was added dropwise. The reaction mixture was stirred for 15 min in the ice-water bath and then was diluted with DCM. The aqueous layer was extracted with DCM. The combined organic layers were dried over Na₂SO₄ and concentrated to dryness to yield crude 5-cyano-2-(trifluoromethyl)benzenesulfonyl chloride, which was used without further purification.

Then, a mixture of the crude product, NaHCO₃ (334 mg, 4.0 mmol), and morpholine (41 μL, 0.48 mmol) in DCM was stirred overnight at room temperature. Upon completion, the solvent was removed under reduced pressure and purification via column chromatography afforded pure product 3-(morpholinosulfonyl)-4-(trifluoromethyl)benzonitrile (46 mg, 30% yield over 3 steps). ¹H NMR (CDCl₃) δ 8.39 (s, 1H), 8.05 (d, J=8.0 Hz, 1H), 8.00 (dd, J=8.2, 0.6 Hz, 1H), 3.74 (t, J=4.8 Hz, 4H), 3.28 (t, J=4.8 Hz).

Lastly, a mixture of the product (20 mg, 0.06 mmol), K₂CO₃ (9 mg, 0.06 mmol), and benzhydrazide (17 mg, 0.12 mmol) in 1-BuOH (63 μL) was stirred for 4 hours at 120° C. in a pre-heated oil bath. Upon completion, the solvent was removed under reduced pressure and purification via prep HPLC afforded pure product SR-28004 (9 mg, 26% yield). ¹H NMR (CD₃OD) δ 8.73 (s, 1H), 8.56 (d, J=8.4 Hz, 1H), 8.25 (d, J=8.4 Hz, 1H), 8.08-8.06 (m, 2H), 7.56 (d, J=5.6 Hz, 3H), 3.73 (t, J=4.6 Hz, 4H), 3.26 (t, J=4.6 Hz, 4H); MS (m/z): [M] calc'd for C₁₉H₁₇F₃N₄O₃S is 438.10. found 438.91 [M+H].

### Example 31. 4-((5-(5-(4-Methoxyphenyl)-4H-1,2,4-triazol-3-yl)-2-methylphenyl)sulfonyl) morpholine (SR-28104)

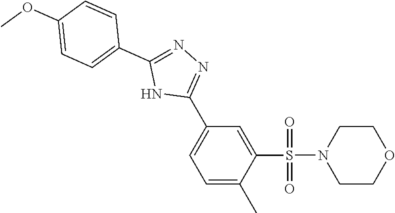 [SMILES: COc1ccc(-c2nnc(-c3ccc(C)c(S(=O)(=O)N4CCOCC4)c3)[nH]2)cc1]

This compound was prepared according to the procedure for SR-25604 with 4-methoxybenzohydrazide in 35% overall yield. ¹H NMR (CD₃OD) δ 8.60 (d, J=2.0 Hz, 1H), 8.24 (dd, J=8.0, 2.0 Hz, 1H), 7.99 (dd, J=6.8, 2.0 Hz, 2H), 7.57 (d, J=8.0 Hz, 1H), 7.09 (dd, J=6.8, 2.0 Hz, 2H), 3.88 (s, 3H), 3.72 (t, J=4.8 Hz, 4H), 3.19 (t, J=4.8 Hz, 4H), 2.70 (s, 3H); MS (m/z): [M] calc'd for C₂₀H₂₂N₄O₄S is 414.14. found 414.96 [M+H].

### Example 32. N,N-dimethyl-4-(5-(4-methyl-3-(morpholinosulfonyl)phenyl)-4H-1,2,4-triazol-3-yl)aniline (SR-28124)

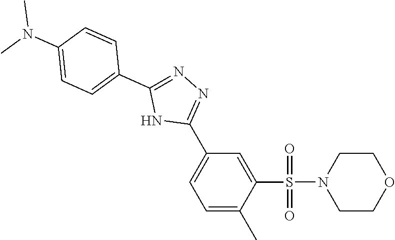 [SMILES: Cc1ccc(-c2nnc(-c3ccc(N(C)C)cc3)[nH]2)cc1S(=O)(=O)N1CCOCC1]

This compound was prepared according to the procedure for SR-25604 with 4-(dimethylamino)benzhydrazide in 18% overall yield. ¹H NMR (CD₃OD) δ 8.60 (d, J=1.6 Hz, 1H), 8.24 (dd, J=7.8, 1.8 Hz, 1H), 7.90 (d, J=8.8 Hz, 2H), 7.57 (d, J=7.6 Hz, 1H), 6.92 (d, J=9.2 Hz, 2H), 3.72 (t, J=4.8 Hz, 4H), 3.19 (t, J=4.8 Hz, 4H), 3.07 (s, 6H), 2.71 (s, 3H); MS (m/z): [M] calc'd for C₂₁H₂₅N₅O₃S is 427.17. found 428.09 [M+H].

### Example 33. 4-((5-(5-(4-Bromophenyl)-4H-1,2,4-triazol-3-yl)-2-methylphenyl)sulfonyl) morpholine (SR-28144)

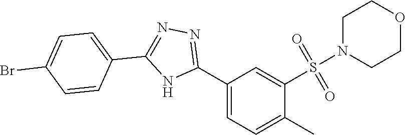 [SMILES: Cc1ccc(-c2nnc(-c3ccc(Br)cc3)[nH]2)cc1S(=O)(=O)N1CCOCC1]

This compound was prepared according to the procedure for SR-25604 with 4-bromobenzohydrazide in 26% overall yield. ¹H NMR (CD₃OD) δ 8.59 (d, J=1.6 Hz, 1H), 8.23 (d, J=7.6 Hz, 1H), 7.99 (d, J=8.4 Hz, 2H), 7.70 (d, J=8.4 Hz, 2H), 7.58 (d, J=8.0 Hz, 1H), 3.72 (t, J=4.6 Hz, 4H), 3.19 (t, J=4.8 Hz, 4H), 2.71 (s, 3H); MS (m/z): [M] calc'd for C₁₉H₁₉BrN₄O₃S is 462.04/464.04. found 462.88/464.89 [M+H].

### Example 34. 4-((2-Methyl-5-(5-(pyridin-4-yl)-4H-1,2,4-triazol-3-yl)phenyl)sulfonyl) morpholine (SR-28224)

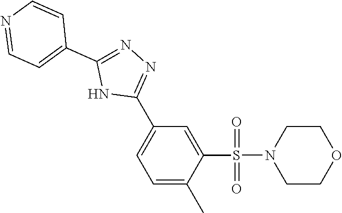 [SMILES: Cc1ccc(-c2nnc(-c3ccncc3)[nH]2)cc1S(=O)(=O)N1CCOCC1]

This compound was prepared according to the procedure for SR-25604 with isonicotinic hydrazide in 15% overall yield. ¹H NMR (CD₃OD) δ 8.86 (br, 2H), 8.62 (d, J=2.0 Hz, 1H), 8.53 (br, 2H), 8.24 (dd, J=8.0, 1.6 Hz, 1H), 7.65 (d, J=8.0 Hz, 1H), 3.72 (t, J=4.8 Hz, 4H), 3.19 (t, J=4.8 Hz, 4H), 2.73 (s, 3H); MS (m/z): [M] calc'd for C₁₈H₁₉N₅O₃S is 385.12. found 385.96 [M+H].

### Example 35. 4-((5-(5-Cyclohexyl-4H-1,2,4-triazol-3-yl)-2-methylphenyl)sulfonyl)morpholine (SR-28565)

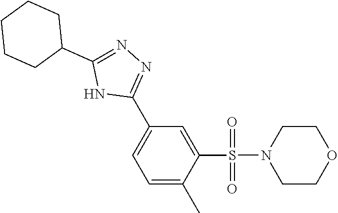 [SMILES: Cc1ccc(-c2nnc(C3CCCCC3)[nH]2)cc1S(=O)(=O)N1CCOCC1]

This compound was prepared according to the procedure for SR-25604 with cyclohexanebenzhydrazide in 30% overall yield. ¹H NMR (CD₃OD) δ 8.51 (d, J=1.6 Hz, 1H), 8.15 (dd, J=7.6, 1.6 Hz, 1H), 7.53 (d, J=8.0 Hz, 1H), 3.70 (t, J=4.8 Hz, 4H), 3.17 (t, J=4.8 Hz, 4H), 2.89 (tt, J=11.8, 3.4 Hz, 1H), 2.68 (s, 3H), 2.06 (dd, J=13.6, 2.0 Hz, 2H), 1.89 (dt, J=12.8, 3.2 Hz, 2H), 1.81-1.77 (m, 1H), 1.63 (qd, J=12.4, 2.8 Hz, 2H), 1.52-1.29 (m, 3H); MS (m/z): [M] calc'd for C₁₉H₂₆N₄O₃S is 390.17. found 390.96 [M+H].

### Example 36. 4-((2-Methyl-5-(1-phenyl-1H-pyrazol-4-yl)phenyl)sulfonyl)morpholine (SR-28925)

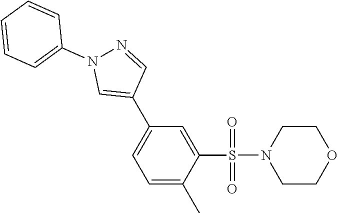 [SMILES: Cc1ccc(-c2cnn(-c3ccccc3)c2)cc1S(=O)(=O)N1CCOCC1]

A mixture of 4-((2-methyl-5-(1-(methylsulfonyl)-1H-pyrazol-4-yl)phenyl)sulfonyl)morpholine (52 mg, 0.17 mmol), Cs₂CO₃ (110 mg, 0.34 mmol), N,N′-dimethylethylenediamine (3.6 μL, 0.034 mmol), iodobenzene (38 μL, 0.34 mmol), and CuI (1.6 mg, 0.008 mmol) in DMF (850 μL) was stirred for 48 hours at 120° C. in a pre-heated oil bath. Upon completion, solvent was removed under reduced pressure and purification via column chromatography afforded pure product SR-28925 (46 mg, 71% yield). ¹H NMR (CD₃OD) δ 8.71 (s, 1H), 8.12 (t, J=2.2 Hz, 2H), 7.86-7.81 (m, 3H), 7.51 (t, J=8.0 Hz, 2H), 7.45 (d, J=8.0 Hz, 1H), 7.35 (t, J=7.4 Hz, 1H), 3.70 (t, J=4.6 Hz, 4H), 3.15 (t, J=4.6 Hz, 4H), 2.64 (s, 3H); MS (m/z): [M] calc'd for C₂₀H₂₁N₃O₃S is 383.13. found 384.08 [M+H].

### Example 37. 4-((2-Methyl-5-(5-phenyl-1H-imidazol-2-yl)phenyl)sulfonyl)morpholine (SR-28804)

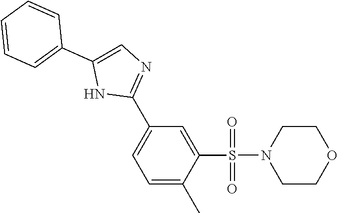 [SMILES: Cc1ccc(-c2ncc(-c3ccccc3)[nH]2)cc1S(=O)(=O)N1CCOCC1]

A mixture of 3-(chlorosulfonyl)-4-methylbenzoic acid (1 g, 4.3 mmol) and morpholine (1.5 mL, 17 mmol) in DCM was stirred overnight at room temperature. Upon completion, the reaction mixture was quenched with 10% HCl_{(aq)}. The aqueous layer was extracted with DCM. The combined organic layers were dried over Na₂SO₄ and concentrated to dryness to yield crude 4-methy-3-(morpholinosulfonyl)benzoic acid, which was used without further purification. MS (m/z): [M] calc'd for C₁₂H₁₅NO₅S is 285.07. found 285.77 [M+H].

Then, a mixture of 4-methyl-3-(morpholinosulfonyl)benzoic acid (50 mg, 0.18 mmol), 2-aminoacetophenon HCl (36 mg, 0.21 mmol), DIPEA (61 μL, 0.35 mmol), and HATU (67 mg, 0.18 mmol) in DMF stirred overnight at room temperature. Upon completion, the solvent was removed under reduced pressure and purification via column chromatography afforded 4-methyl-3-(morpholinosulfonyl)-N-(2-oxo-2-phenylethyl)benzamide (115 mg, >100% yield). MS (m/z): [M] calc'd for C₂₀H₂₂N₂O₅S is 402.12 found 402.79 [M+H].

Lastly, a mixture of 4-methyl-3-(morpholinosulfonyl)-N-(2-oxo-2-phenylethyl)benzamide (115 mg, 0.29 mmol) and NH₄OAc (265 mg, 3.4 mmol) in xylenes was stirred overnight at 135° C. in a pre-heated oil bath. Upon completion, the solvent was removed under reduced pressure and purification via prep HPLC afforded pure product SR-28804 as a TFA salt (36 mg, 41% yield over 2 steps). ¹H NMR (CD₃OD) δ 8.57 (s, 1H), 8.16 (dd, J=8.0, 2.0 Hz, 1H), 7.98 (s, 1H), 7.85-7.82 (m, 2H), 7.30 (d, J=8.0 Hz, 1H), 7.62-7.46 (m, 3H), 3.72 (t, J=4.8 Hz, 4H), 3.21 (t, J=4.6 Hz, 4H), 2.75 (s, 3H); MS (m/z): [M] calc'd for C₂₀H₂₁N₃O₃S is 383.13 found 383.96 [M+H].

### Example 38. 4-((5-(1-Benzyl-1H-pyrazol-4-yl)-2-methylphenyl)sulfonyl)morpholine (SR-28764)

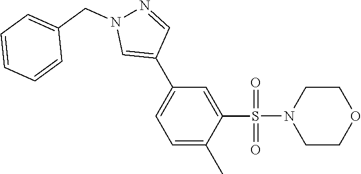 [SMILES: Cc1ccc(-c2cnn(Cc3ccccc3)c2)cc1S(=O)(=O)N1CCOCC1]

A mixture of 4-((5-bromo-2-methylphenyl)sulfonyl)morpholine (25 mg, 0.08 mmol), 1-benzyl-pyrazole-4-boronic acid pinacol ester (33 mg, 0.12 mmol) and Na₂CO_{3(aq)} (117 μL, 2M, 0.23 mmol) in 1,4-dioxane (1.6 mL) was de-gassed and back-filled with argon 3 times at room temperature in a microwave vial. Pd(PPh₃)₄ (4.5 mg, 0.004 mmol) was added, the vial was sealed, and the mixture was de-gassed and back-filled with argon. Then, the reaction mixture was microwaved in a Biotage© microwave reactor at 120° C. for 30 min under normal absorption conditions. Upon completion, solvent was removed under reduced pressure and purification via prep HPLC afforded pure product SR-28764 (12 mg, 39% yield). ¹H NMR (CD₃OD) δ 8.14 (s, 1H), 7.99 (d, J=2.0 Hz, 1H), 7.90 (s, 1H), 7.71 (dd, J=7., 1.8 Hz, 1H), 7.39 (d, J=8.0 Hz, 1H), 7.36-7.26 (m, 5H), 5.36 (s, 2H), 3.67 (t, J=4.8 Hz, 4H), 3.11 (t, J=4.8 Hz, 4H), 2.60 (s, 3H); MS (m/z): [M] calc'd for C₂₁H₂₃N₃O₃S is 397.15 found 397.88 [M+H].

### Example 39. 4-((2-Methyl-5-(4-methyl-5-phenyl-4H-1,2,4-triazol-3-yl)phenyl)sulfonyl) morpholine (SR-28864)

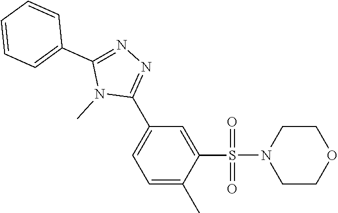 [SMILES: Cc1ccc(-c2nnc(-c3ccccc3)n2C)cc1S(=O)(=O)N1CCOCC1]

A mixture of 4-methy-3-(morpholinosulfonyl)benzoic acid (90 mg, 0.32 mmol), DMF (2 drops), and SOCl₂ (50 μL, 0.69 mmol) in DCM was stirred overnight at room temperature. Upon completion, solvent was removed under reduced pressure. The remaining residue was re-dissolved in DCM. Methylamine HCl (26 mg, 0.39 mmol) and NaHCO₃ (265 mg, 3.2 mmol) were added to the solution and the reaction mixture was stirred overnight at room temperature. Upon completion, solvent was removed under reduced pressure and purification via column chromatography afforded N,4-dimethy-3-(morpholinosulfonyl)benzamide (72 mg, 77% yield over 2 steps). MS (m/z): [M] calc'd for C₁₃H₁₈N₂O₄S is 298.36. found 298.88 [M+H].

Then, a mixture of the product (72 mg, 0.24 mmol) and 2-fluoropyridine (24 μL, 0.28 mmol) in DCE (804 μL) was cooled in an ice-water bath under argon. Trifluoromethane sulfonic anhydride (45 μL, 0.27 mmol) was added dropwise and the reaction mixture was stirred for 10 min in the ice-water bath under argon. Benzhydrazide (36 mg, 0.26 mmol) was added, and the reaction mixture was stirred for 10 min at room temperature. Then, the reaction mixture was microwaved in a Biotage© microwave reactor at 140° C. for 2 hours under very high absorption conditions. Upon completion, solvent was removed under reduced pressure and purification via prep HPLC afforded pure product SR-28864 (16 mg, 17% yield). ¹H NMR (CD₃OD) δ 8.35 (d, J=2.0 Hz, 1H), 8.03 (dd, J=7.8, 1.8 Hz, 1H), 7.86 (dt, J=6.4, 1.6 Hz, 2H), 7.78-7.69 (m, 4H), 3.87 (s, 3H), 3.72 (t, J=4.6 Hz, 4H), 3.22 (t, J=4.8 Hz, 4H), 2.78 (s, 3H); MS (m/z): [M] calc'd for C₂₀H₂₂N₄O₃S is 398.14. found 399.20 [M+H].

### Example 40. 4-((2-Methyl-5-(1-phenyl-1H-pyrrol-3-yl)phenyl)sulfonyl)morpholine (SR-29084)

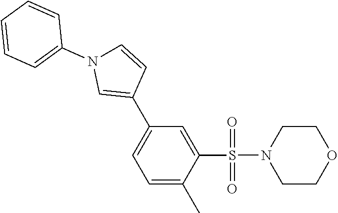 [SMILES: Cc1ccc(-c2ccn(-c3ccccc3)c2)cc1S(=O)(=O)N1CCOCC1]

A mixture of 4-((5-bromo-2-methylphenyl)sulfonyl)morpholine (100 mg, 0.31 mmol), 1-(triisopropylsilyl)pyrroleboronic acid (100 mg, 0.37 mmol), de-ionized water (800 μL), and K₂CO₃ (108 mg, 0.78 mmol) in DME (3.2 mL) was de-gassed and back-filled with argon 3 times at room temperature in a microwave vial. Pd(PPh₃)₄ (18 mg, 0.015 mmol) was added, the vial was sealed, and the mixture was de-gassed and back-filled with argon. Then, the reaction mixture was microwaved in a Biotage© microwave reactor at 110° C. for 4 hours under normal absorption conditions. Upon completion, solvent was removed under reduced pressure and purification via column chromatography afforded pure product 4-((2-methyl-5-(1-(triisopropylsilyl)-1H-pyrrol-3-yl)phenyl)sulfonyl)morpholine (42 mg, 29% yield). MS (m/z): [M] calc'd for C₂₄H₃₈N₂O₃SSi is 462.24 found 463.30 [M+H].

Then, a mixture of the product (42 mg, 0.09 mmol) and TBAF (109 μL, 1M in THF) in THF stirred for 5 min at room temperature under argon. Upon completion, solvent was removed under reduced pressure and purification via column chromatography afforded pure product 4-((2-methyl-5-(1H-pyrrol-3-yl)phenyl)sulfonyl)morpholine (22 mg, 79% yield). MS (m/z): [M] calc'd for C₁₅H₁₈N₂O₃S is 306.10. found 306.85 [M+H].

Finally, a mixture of the product (22 mg, 0.07 mmol), Cs₂CO₃ (47 mg, 0.14 mmol), iodobenzene (16 μL, 0.14 mmol), N,N′-dimethylethylenediamine (1.5 μL, 0.014 mmol), and CuI (1.0 mg, 0.005 mmol) in DMF (361 μL) was stirred for 48 hours at 120° C. in a pre-heated oil bath. Upon completion, solvent was removed under reduced pressure and purification via column chromatography afforded pure product SR-29084 (6 mg, 22% yield). ¹H NMR (CD₃OD) δ 8.05 (s, 1H), 7.78 (d, J=6.4 Hz, 1H), 7. 66 (s, 1H), 7.55 (d, J=4.8 Hz, 2H), 7.50-7.47 (m, 2H), 7.39 (dd, J=8.0, 3.2 Hz, 1H), 7.28 (m, 2H), 6.68 (s, 1H), 3.70 (d, J=4.0 Hz, 4H), 3.14 (d, J=4.0 Hz, 4H), 2.62 (d, J=3.2 Hz, 3H); MS (m/z): [M] calc'd for C₂₁H₂₂N₂O₃S is 382.14 found 382.97.

### Example 41. 4-((2-Methyl-5-(2-phenylpyridin-4-yl)phenyl)sulfonyl)morpholine (SR-28984)

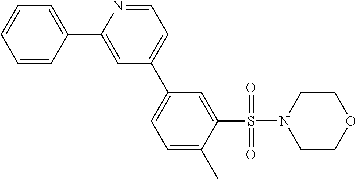 [SMILES: Cc1ccc(-c2ccnc(-c3ccccc3)c2)cc1S(=O)(=O)N1CCOCC1]

A mixture of 4-((5-(2-Chloropyridin-4-yl)-2-methylphenyl)sulfonyl)morpholine (35 mg, 0.1 mmol), phenylboronic acid (18 mg, 0.15 mmol), de-ionized water (250 μL), and K₂CO₃ (34 mg, 0.25 mmol) in 1,4-dioxane (1 mL) was de-gassed and back-filled with argon 3 times at room temperature in a microwave vial. Pd(dppf)Cl₂·CH₂Cl₂ (7.2 mg, 0.009 mmol) was added, the vial was sealed, and the mixture was de-gassed and back-filled with argon. Then, the reaction mixture was microwaved in a Biotage© microwave reactor at 120° C. for 2 hours under normal absorption conditions. Upon completion, solvent was removed under reduced pressure and purification via prep HPLC followed by column chromatography afforded pure product SR-28984 (36 mg, 92% yield). ¹H NMR (CD₃OD) δ 8.70 (d, J=5.2 Hz, 1H), 8.26 (d, J=1.6 Hz, 1H), 8.20 (d, J=0.8 Hz, 1H), 8.04-8.02 (m, 3H), 7.67 (dd, J=5.2, 1.2 Hz, 1H), 7.62 (d, J=8.0 Hz, 1H), 7.54-7.46 (m, 3H), 3.71 (t, J=4.6 Hz, 4H), 3.18 (t, J=4.6 Hz, 4H), 2.72 (s, 3H); MS (m/z): [M] calc'd for C₂₂H₂₂N₂O₃S is 394.14, found 395.30 [M+H].

### Example 42. 4-((5-(2-(4-Fluorophenyl)pyridin-4-yl)-2-methylphenyl)sulfonyl)morpholine (SR-28924)

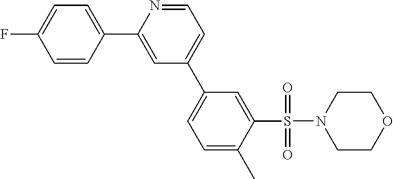 [SMILES: Cc1ccc(-c2ccnc(-c3ccc(F)cc3)c2)cc1S(=O)(=O)N1CCOCC1]

This compound was prepared according to the procedure for SR-28984 with 4-fluorophenylboronic acid pinacol ester in 86% yield. ¹H NMR (CD₃OD) δ 8.67 (d, J=5.2 Hz, 1H), 8.24 (d, J=1.6 Hz, 1H), 8.09-8.05 (m, 3H), 8.01 (dd, J=8.0, 2.0 Hz, 1H), 7.64 (dd, J=5.2, 1.6 Hz, 1H), 7.60 (d, J=7.6 Hz, 1H), 7.26-7.22 (m, 2H), 3.70 (t, J=4.6 Hz, 4H), 3.17 (t, J=4.8 Hz, 4H), 2.70 (s, 3H); MS (m/z): [M] calc'd for C₂₂H₂₁FN₂O₃S is 412.13. found 413.05 [M+H].

### Example 43. 4-((2-Methyl-5-(5-phenylfuran-3-yl)phenyl)sulfonyl)morpholine (SR-29044)

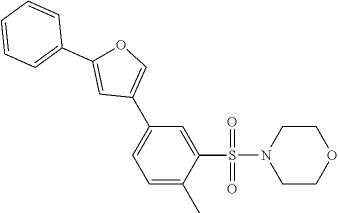 [SMILES: Cc1ccc(-c2coc(-c3ccccc3)c2)cc1S(=O)(=O)N1CCOCC1]

A mixture of 4-((5-bromo-2-methylphenyl)sulfonyl)morpholine (50 mg, 0.16 mmol), 3-furanylboronic acid (26 mg, 0.23 mmol), de-ionized water (400 μL), and K₂CO₃ (65 mg, 0.47 mmol) in 1,4-dioxane (1.6 mL) was de-gassed and back-filled with argon 3 times at room temperature in a microwave vial. Pd(PPh₃)₄ (9 mg, 0.008 mmol) was added, the vial was sealed, and the mixture was de-gassed and back-filled with argon. Then, the reaction mixture was microwaved in a Biotage© microwave reactor at 120° C. for 2 hours under normal absorption conditions. Upon completion, solvent was removed under reduced pressure and purification via column chromatography afforded product 4-((5-(furan-3-yl)-2-methylphenyl)sulfonyl)morpholine (55 mg). MS (m/z): [M] calc'd for C₁₅H₁₇NO₄S is 307.09. found 307.87 [M+H].

Then a mixture of the product (55 mg) and Br₂ (10 μL, 0.19 mmol) in Et₂O was stirred overnight at room temperature. Upon completion, solvent was removed under reduced pressure and purification via column chromatography afforded pure product 4-((5-(5-bromofuran-3-yl)-2-methylphenyl)sulfonyl)morpholine (51 mg, 85% yield over 2 steps). MS (m/z): [M] calc'd for C₁₅H₁₆BrNO₄S is 385.00/387.00. found 385.81/387.81 [M+1].

Finally, a mixture of the product (51 mg, 0.13 mmol), phenylboronic acid (24 mg, 0.2 mmol), de-ionized water (333 μL), and K₂CO₃ (45 mg, 0.33 mmol) in 1,4-dioxane (1.3 mL) was de-gassed and back-filled with argon 3 times at room temperature in a microwave vial. Pd(dppf)Cl₂·CH₂Cl₂ (9.6 mg, 0.013 mmol) was added, the vial was sealed, and the mixture was de-gassed and back-filled with argon. Then, the reaction mixture was microwaved in a Biotage© microwave reactor at 120° C. for 2 hours under normal absorption conditions. Upon completion, solvent was removed under reduced pressure and purification via prep HPLC afforded pure product SR-29044 (22 mg, 43% yield). ¹H NMR (CD₃OD) δ 7.76 (d, J=2.0 Hz, 1H), 7.64 (d, J=1.6 Hz, 1H), 7.58 (dd, J=7.8, 1.8 Hz, 1H), 7.45-7.42 (m, 3H), 7.35-7.29 (m, 3H), 6.67 (d, J=1.6 Hz, 1H), 3.64 (t, J=4.8 Hz, 4H), 3.01 (t, J=4.8 Hz, 4H), 2.64 (s, 3H); MS (m/z): [M] calc'd for C₂₁H₂₁NO₄S is 383.12. found 383.87 [M+1].

### Example 44. 4-((2-Methyl-5-(5-(4-nitrophenyl)-4H-1,2,4-triazol-3-yl)phenyl)sulfonyl) morpholine (SR-29045)

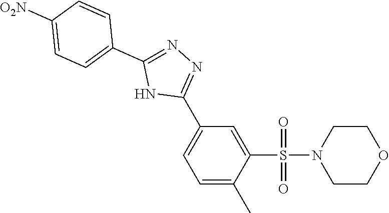 [SMILES: Cc1ccc(-c2nnc(-c3ccc([N+](=O)[O-])cc3)[nH]2)cc1S(=O)(=O)N1CCOCC1]

This compound was prepared according to the procedure for SR-25604 with 4-nitrobenzhydrazide in 15% overall yield. ¹H NMR (CD₃OD) δ 8.61 (d, J=1.6 Hz, 1H), 8.36 (q, J=8.8 Hz, 4H), 8.24 (d, J=8.0 Hz, 1H), 7.62 (d, J=8.0 Hz, 1H), 3.72 (t, J=4.6 Hz, 4H), 3.20 (t, J=4.6 Hz, 4H), 2.72 (s, 3H); MS (m/z): [M] calc'd for C₉H₁₉N₅O₅S is 429.11. found 429.96 [M+1].

### Example 45. 4-((2-Methyl-5-(5-phenylthiophen-3-yl)phenyl)sulfonyl)morpholine (SR-29184)

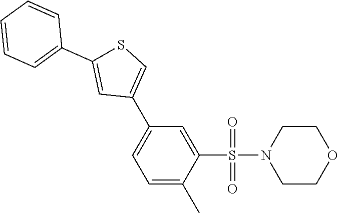 [SMILES: Cc1ccc(-c2csc(-c3ccccc3)c2)cc1S(=O)(=O)N1CCOCC1]

This compound was prepared according to the procedure for SR-29044 with thiophene-3-boronic acid in 35% overall yield. ¹H NMR (CD₃OD) δ 7.57-7.55 (m, 2H), 7.51 (d, J=5.6 Hz, 1H), 7.40 (d, J=7.6 Hz, 1H), 7.32-7.25 (m, 5H), 7.21 (d, J=5.2 Hz, 1H), 3.61 (t, J=4.8 Hz, 4H), 2.83 (t, J=4.8 Hz, 4H), 2.60 (s, 3H); MS (m/z): [M] calc'd for C₂₁H₂₁NO₃S₂ is 399.10. found 399.85 [M+1].

### Example 46. 4-((5-([2,4′-Bipyridin]-4-yl)-2-methylphenyl)sulfonyl)morpholine (SR-29204)

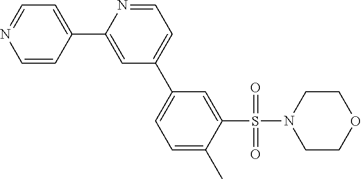 [SMILES: Cc1ccc(-c2ccnc(-c3ccncc3)c2)cc1S(=O)(=O)N1CCOCC1]

This compound was prepared according to the procedure for SR-28984 with 4-pyridineboronic acid pinacol ester in 71% yield. ¹H NMR (CD₃OD) δ, 8.79 (dd, J=5.2, 0.8 Hz, 1H), 8.69 (br, 2H), 8.29-8.28 (m, 2H), 8.15 (d, J=6.0 Hz, 2H), 8.05 (dd, J=8.0, 2.0 Hz, 1H), 7.78 (dd, J=5.2, 1.6 Hz, 1H), 7.63 (d, J=8.0 Hz, 1H), 3.71 (t, J=4.8 Hz, 4H), 3.18 (t, J=4.8 Hz, 4H), 2.72 (s, 3H); MS (m/z): [M] calc'd for C₂₁H₂₁N₃O₃S is 395.13. found 395.99 [M+1].

### Example 47. 4-(4-(4-Methyl-3-(morpholinosulfonyl)phenyl)pyridin-2-yl)morpholine (SR-29224)

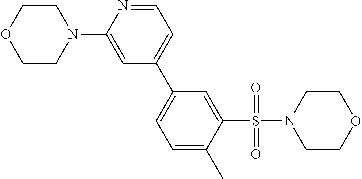 [SMILES: Cc1ccc(-c2ccnc(N3CCOCC3)c2)cc1S(=O)(=O)N1CCOCC1]

A mixture of 4-((5-bromo-2-methylphenyl)sulfonyl)morpholine (20 mg, 0.06 mmol), K₂CO₃ (22 mg, 0.16 mmol), de-ionized water (300 μL), and 2-morpholinopyridine-4-boronic acid pinacol ester (27 mg, 0.09 mmol) in 1,4-dioxane (1.2 mL) was de-gassed and back-filled with argon 3 times at room temperature in a microwave vial. Pd(PPh₃)₄ (3.6 mg, 0.003 mmol) was added, the vial was sealed, and the mixture was de-gassed and back-filled with argon. Then, the reaction mixture was microwaved in a Biotage© microwave reactor at 120° C. for 2 hours under normal absorption conditions. Upon completion, solvent was removed under reduced pressure and purification via prep HPLC followed by column chromatography afforded pure product SR-29224 (25 mg, 100% yield). ¹H NMR (CDCl₃) δ 8.22 (d, J=1.6 Hz, 1H), 8.09 (d, J=6.4 Hz, 1H), 7.99 (dd, J=7.8, 1.8 Hz, 1H), 7.62 (d, J=8.0 Hz, 1H), 7.41 (s, 1H), 7.22 (d, J=6.4 Hz, 1H), 3.87 (t, J=4.8 Hz, 4H), 3.71-3.68 (m, 8H), 3.17 (t, J=4.8 Hz, 4H), 2.71 (s, 3H); MS (m/z): [M] calc'd for C₂₀H₂₅N₃O₄S is 403.16. found 404.30 [M+1].

### Example 48. 4-((2-Methyl-5-(4-phenyl-1H-pyrrol-3-yl)phenyl)sulfonyl)morpholine (SR-29285)

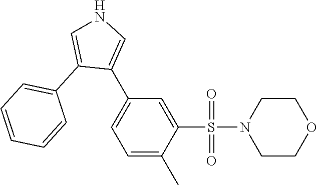 [SMILES: Cc1ccc(-c2c[nH]cc2-c2ccccc2)cc1S(=O)(=O)N1CCOCC1]

This compound was prepared according to the procedure for SR-29044 with 1-(triisopropyl)pyrrole-3-boronic acid pinacol ester in 32% overall yield after TBAF de-protection of the TIPS group. ¹H NMR (CD₃OD) δ 7.51 (s, 1H), 7.48 (d, J=8.0 Hz, 1H), 7.30 (d, J=8.0 Hz, 1H), 7.26-7.14 (m, 5H), 6.95 (d, J=1.2 Hz, 1H), 6.87 (d, J=1.2 Hz, 1H), 3.62 (t, J=4.6 Hz, 4H), 2.88 (t, J=4.6 Hz, 4H), 2.58 (s, 3H); MS (m/z): [M] calc'd for C₂₁H₂₂N₂O₃S is 382.14. found 383.20 [M+1].

### Example 49. 4-((2-Cyclopropyl-5-(5-phenyl-4H-1,2,4-triazol-3-yl)phenyl)sulfonyl) morpholine (SR-29784)

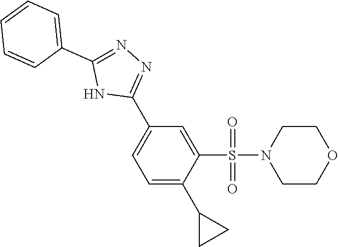 [SMILES: O=S(=O)(c1cc(-c2nnc(-c3ccccc3)[nH]2)ccc1C1CC1)N1CCOCC1]

A mixture of 4-((5-bromo-2-chlorophenyl)sulfonyl)morpholine (100 mg, 0.29 mmol) and Zn(CN)₂ (18 mg, 0.15 mmol) in DMF (2.9 mL) was de-gassed and back-filled with argon 3 times at room temperature in a microwave vial. Pd(PPh₃)₄ (6.4 mg, 0.006 mmol) was added, the vial was sealed, and the mixture was de-gassed and back-filled with argon. Then, the reaction mixture was microwaved in a Biotage© microwave reactor at 90° C. for 60 min under normal absorption conditions. Upon completion, solvent was removed under reduced pressure and purification via column chromatography afforded pure product 4-chloro-3-(morpholinosulfonyl)benzonitrile (73 mg, 87% yield).

Then, a mixture of the product (73 mg, 0.25 mmol), K₂CO₃ (106 mg, 0.77 mmol), cyclopropylboronic acid (33 mg, 0.38 mmol), and de-ionized water (325 μL) in 1,4-dioxane (1.3 mL) was de-gassed and back-filled with argon 3 times at room temperature in a microwave vial. Pd(dppf)Cl₂·CH₂Cl₂ (9.3 mg, 0.013 mmol) was added, the vial was sealed, and the mixture was de-gassed and back-filled with argon. Then, the reaction mixture was microwaved in a Biotage© microwave reactor at 120° C. for 2 hours min under normal absorption conditions. Upon completion, solvent was removed under reduced pressure and purification via column chromatography afforded pure product 4-cyclopropyl-3-(morpholinosulfonyl)benzonitrile (29 mg, 39% yield).

Then, a mixture of the product (29 mg, 0.1 mmol), K₂CO₃ (14 mg, 0.1 mmol), and benzhydrazide (27 mg, 0.2 mmol) in 1-BuOH (99 μL) was stirred at 120° C. in a pre-heated oil bath for 24 hours. Upon completion, solvent was removed under reduced pressure and purification via prep HPLC afforded pure product SR-29784 (19 mg, 47% yield). ¹H NMR (CD₃OD) δ 8.64 (s, 1H), 8.24 (d, J=8.0 Hz, 1H), 8.06 (d, J=6.8 Hz, 2H), 7.54 (d, J=6.4 Hz, 3H), 7.21 (d, J=8.4 Hz, 1H), 3.72 (t, J=4.6 Hz, 4H), 3.20 (t, J=4.8 Hz, 4H), 2.94-2.88 (m, 1H), 1.20 (dt, J=10.0, 5.2 Hz, 2H), 0.97 (t, J=4.6 Hz, 2H); MS (m/z): [M] calc'd for C₂₁H₂₂N₄O₃S is 410.14. found 410.88 [M+1].

### Example 50. 4-((2-Methyl-5-(1-phenyl-1H-1,2,3-triazol-4-yl)phenyl)sulfonyl)morpholine (SR-30084)

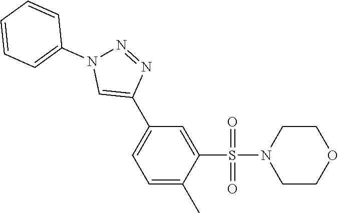 [SMILES: Cc1ccc(-c2cn(-c3ccccc3)nn2)cc1S(=O)(=O)N1CCOCC1]

A mixture of phenylboronic acid (20 mg, 0.16 mmol), NaN₃ (16 mg, 0.25 mmol), and Cu(OAc)₂ (3.0 mg, 0.016 mmol) in MeOH (840 μL) was stirred for 1.5 hours at 55° C. in a pre-heated oil bath and then was cooled to room temperature. 4-((5-Ethynyl-2-methylphenyl)sulfonyl)morpholine (50 mg, 0.19 mmol) and sodium ascorbate (3.7 mg, 0.019 mmol) were added, and the reaction mixture was stirred overnight at room temperature. Upon completion, solvent was removed under reduced pressure and purification via column chromatography afforded pure product SR-30084 (32 mg, 51% yield). ¹H NMR (CDCl₃) δ 8.33 (s, 1H), 8.28 (d, J=1.2 Hz, 1H), 8.12 (d, J=7.6 Hz, 1H), 7.80 (d, J=8.4 Hz, 2H), 7.56 (td, J=6.8, 1.2 Hz, 2H), 7.49-7.44 (m, 2H), 3.744 (d, J=4.4 Hz, 4H), 3.20 (d, J=4.4 Hz, 4H), 2.68 (s, 3H); MS (m/z): [M] calc'd for C₁₉H₂₀N₄O₃S is 384.13. found 384.80 [M+1].

### Example 51. (cis)-2,6-dimethyl-4-((5-(5-phenyl-4H-1,2,4-triazol-3-yl)-2-(trifluoromethyl) phenyl)sulfonyl)morpholine (SR-30786)

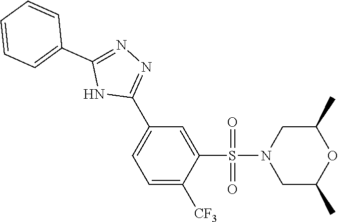 [SMILES: C[C@@H]1CN(S(=O)(=O)c2cc(-c3nnc(-c4ccccc4)[nH]3)ccc2C(F)(F)F)C[C@H](C)O1]

A mixture of 2-(trifluoromethyl)benzene sulfonyl chloride (100 mg, 0.41 mmol), NaHCO₃ (343 mg, 4.1 mmol), and cis-2,6-dimethylmorpholine (60 μL, 0.49 mmol) in DCM was stirred overnight at room temperature. Upon completion, the solvent was removed under reduced pressure and purification via column chromatography afforded product (cis)-2,6-dimethyl-4-((2-(trifluoromethyl)phenyl)sulfonyl)morpholine (187 mg). MS (m/z): [M] calc'd for C₁₃H₁₆F₃NO₃S is 323.08. found 323.77 [M+1].

Then, a mixture of the product (187 mg) and NBS (119 mg, 0.67 mmol) in concentrated H₂SO₄ (512 μL) stirred overnight at room temperature then was diluted with brine. The aqueous layer was extracted with DCM. The combined organic layers were dried over Na₂SO₄ and concentrated to dryness. Purification via column chromatography afforded pure product (cis)-4-((5-bromo-2-(trifluoromethyl)phenyl)sulfonyl)-2,6-dimethylmorpholine (50 mg, 30% yield over 2 steps). ¹H NMR (CDCl₃) δ 8.02 (d, J=1.6 Hz, 1H), 7.95 (d, J=8.8 Hz, 1H), 7.85 (dd, J=8.4, 1.6 Hz, 1H), 3.70-3.62 (m, 2H), 3.56 (d, J=12.0 Hz, 2H), 2.38 (t, J=11.2 Hz, 2H), 1.15 (d, J=6.0 Hz, 6H).

Then, a mixture of the product (50 mg, 0.12 mmol), dppf (3.8 mg, 0.007 mmol), de-ionized water (25 μL), and Zn(CN)₂ (18 mg, 0.15 mmol) in DMF (2.5 mL) was de-gassed and back-filled with argon 3 times at room temperature in a microwave vial. Pd₂(dba)₃ (2.5 mg, 0.003 mmol) was added, the vial was sealed, and the mixture was de-gassed and back-filled with argon. Then, the reaction mixture was microwaved in a Biotage© microwave reactor at 115° C. for 30 min under normal absorption conditions. Upon completion, solvent was removed under reduced pressure and purification via column chromatography afforded pure product 3-(((cis)-2,6-dimethylmorpholino)sulfonyl)-4-(trifluoromethyl)benzonitrile (38 mg, 88% yield).

Finally, a mixture of the product (38 mg, 0.11 mmol), K₂CO₃ (15 mg, 0.11 mmol), and benzhydrazide (30 mg, 0.22 mmol) in 1-BuOH (109 μL) was stirred at 120° C. in a pre-heated oil bath for 24 hours. Upon completion, solvent was removed under reduced pressure and purification via prep HPLC afforded pure product SR-30786 (28 mg, 55% yield). ¹H NMR (CD₃OD) δ 8.71 (s, 1H), 8.55 (d, J=8.0 Hz, 1H), 8.23 (d, J=8.4 Hz, 1H), 8.06 (d, J=5.6 Hz, 2H), 7.57-7.55 (m, 3H), 3.69-3.64 (m, 4H), 2.42 (t, J=11.8 Hz, 2H), 1.15 (d, J=6.0 Hz, 6H); ¹⁹F NMR (CD₃OD) δ −58.52; MS (m/z): [M] calc'd for C₂₁H₂₁F₃N₄O₃S is 466.13. found 466.93 [M+1].

### Example 52. 4-((5-(5-Phenyl-4H-1,2,4-triazol-3-yl)-2-(trifluoromethoxy)phenyl)sulfonyl) morpholine (SR-31545)

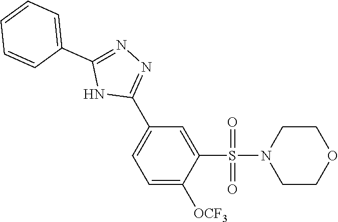 [SMILES: O=S(=O)(c1cc(-c2nnc(-c3ccccc3)[nH]2)ccc1OC(F)(F)F)N1CCOCC1]

This compound was prepared according to the procedure for SR-30786 in 31% overall yield starting with morpholine, rather than cis-2,6-dimethylmorpholine, and 2-(trifluoromethoxy)benzene sulfonyl chloride. ¹H NMR (CD₃OD) δ 8.71 (d, J=1.6 Hz, 1H), 8.49 (d, J=8.0 Hz, 1H), 8.05 (m, 2H), 7.70 (d, J=8.4 Hz, 1H), 7.55 (m, 3H), 3.72 (t, J=4.6 Hz, 4H), 3.24 (t, J=4.6 Hz, 4H); MS (m/z): [M] calc'd for C₁₉H₁₇F₃N₄O₄S is 454.09. found 454.70 [M+1].

### Example 53. 2-Methyl-5-(5-phenyl-4H-1,2,4-triazol-3-yl)-N-(pyridin-3-ylmethyl)benzene sulfonamide (SR-31584)

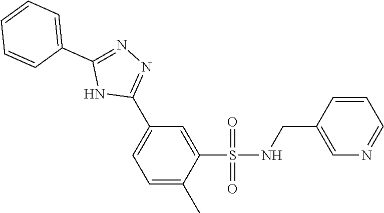 [SMILES: Cc1ccc(-c2nnc(-c3ccccc3)[nH]2)cc1S(=O)(=O)NCc1cccnc1]

This compound was prepared according to the procedure for SR-25604 in 69% overall yield starting with 3-(aminomethyl)pyridine and 5-bromo-2-methylbenzene sulfonyl chloride. ¹H NMR (CD₃OD) δ 8.60 (s, 1H), 8.48-8.42 (br, 2H), 8.16 (dd, J=8.0, 2.0 Hz, 1H), 8.06 (dd, J=7.8, 1.8 Hz, 2H), 7.90 (d, J=7.6 Hz, 1H), 7.57-7.43 (m, 5H), 4.28 (s, 2H), 2.67 (s, 3H); MS (m/z): [M] calc'd for C₂₁H₁₉N₅O₂S is 405.13. found 404.20 [M+1].

### Example 54. 2-Methyl-N-(2-morpholinoethyl)-5-(5-phenyl-4H-1,2,4-triazol-3-yl)benzene sulfonamide (SR-31824)

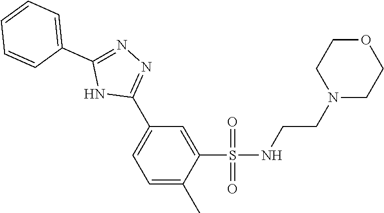 [SMILES: Cc1ccc(-c2nnc(-c3ccccc3)[nH]2)cc1S(=O)(=O)NCCN1CCOCC1]

This compound was prepared according to the procedure for SR-25604 in 68% overall yield starting with 4-(2-aminoethyl)morpholine and 5-bromo-2-methylbenzene sulfonyl chloride. ¹H NMR (CD₃OD) δ 8.67 (d, J=1.6 Hz, 1H), 8.20 (dd, J=7.8, 1.8 Hz, 1H), 8.07 (dd, J=7.6, 1.6 Hz, 2H), 7.56-7.51 (m, 4H), 3.58 (t, J=4.8 Hz, 4H), 3.12 (t, J=6.4 Hz, 2H), 2.74 (s, 3H), 2.39 (t, J=6.6 Hz, 2H), 2.32 (t, J=4.4 Hz, 4H); MS (m/z): [M] calc'd for C₂₁H₂₅N₅O₃S is 427.17. found 428.10 [M+1].

### Example 55. (2-Methyl-5-(5-phenyl-4H-1,2,4-triazol-3-yl)phenyl)(morpholino)methanone (SR-32044)

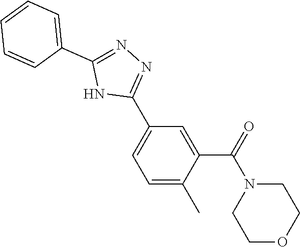 [SMILES: Cc1ccc(-c2nnc(-c3ccccc3)[nH]2)cc1C(=O)N1CCOCC1]

A mixture of 5-bromo-2-methylbenzoic acid (50 mg, 0.23 mmol), DIPEA (81 μL, 0.46 mmol), morpholine (20 μL, 0.23 mmol), and HATU (106 mg, 0.28 mmol) in DMF was stirred overnight at 50° C. Upon completion, the solvent was removed under reduced pressure and purification via column chromatography afforded product (5-bromo-2-methylphenyl)(morpholino)methanone (88 mg). MS (m/z): [M] calc'd for C₁₂H₁₄BrNO₂ is 283.02/285.02. found 284.10/286.10.

Then, a mixture of the product (88 mg), dppf (7.1 mg, 0.013 mmol), de-ionized water (12 μL), and Zn(CN)₂ (32 mg, 0.27 mmol) in DMF (1.2 mL) was de-gassed and back-filled with argon 3 times at room temperature in a microwave vial. Pd₂(dba)₃ (4.7 mg, 0.005 mmol) was added, the vial was sealed, and the mixture was de-gassed and back-filled with argon. Then, the reaction mixture was microwaved in a Biotage© microwave reactor at 115° C. for 30 min under normal absorption conditions. Upon completion, solvent was removed under reduced pressure and purification via column chromatography afforded pure product 4-methyl-3-(morpholine-4-carbonyl)benzonitrile (44 mg, 82% yield over 2 steps).

Finally, a mixture of the product (44 mg, 0.19 mmol), K₂CO₃ (26 mg, 0.19 mmol), and benzhydrazide (52 mg, 0.38 mmol) in 1-BuOH (191 μL) was stirred at 150° C. in a pre-heated oil bath for 24 hours. Upon completion, solvent was removed under reduced pressure and purification via prep HPLC followed by column chromatography afforded pure product SR-32044 (53 mg, 80% yield). ¹H NMR (CD₃OD) δ 8.04 (m, 3H), 7.93 (s, 1H), 7.51-7.44 (m, 4H), 3.86-3.61 (m, 8H), 2.37 (s, 3H); MS (m/z): [M] calc'd for C₂₀H₂₀N₄O₂ is 348.16. found 348.70 [M+1].

### Example 56. N-(1-hydroxy-2-methylpropan-2-yl)-2-methyl-5-(5-phenyl-4H-1,2,4-triazol-3-yl)benzenesulfonamide (SR-32184)

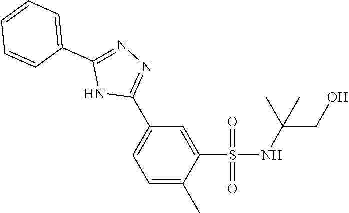 [SMILES: Cc1ccc(-c2nnc(-c3ccccc3)[nH]2)cc1S(=O)(=O)NC(C)(C)CO]

This compound was prepared according to the procedure for SR-25604 in 26% overall yield starting with 2-amino-2-methylpropanol and 5-bromo-2-methylbenzene sulfonyl chloride. ¹H NMR (CD₃OD) δ 8.73 (s, 1H), 8.19 (d, J=8.0 Hz, 1H), 8.07 (d, J=6.4 Hz, 2H), 7.54-7.51 (m, 4H), 3.42 (s, 2H), 2.74 (s, 3H), 1.16 (s, 6H); MS (m/z): [M] calc'd for C₁₉H₂₂N₄O₃S is 386.14. found 387.10 [M+1].

### Example 57. 1-((2-Methyl-5-(5-phenyl-4H-1,2,4-triazol-3-yl)phenyl)sulfonyl)piperidin-4-ol (SR-32045)

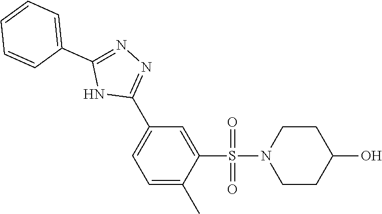 [SMILES: Cc1ccc(-c2nnc(-c3ccccc3)[nH]2)cc1S(=O)(=O)N1CCC(O)CC1]

This compound was prepared according to the procedure for SR-25604 in 53% overall yield starting with 4-hydroxypiperidine and 5-bromo-2-methylbenzene sulfonyl chloride. ¹H NMR (CD₃OD) δ 8.61 (s, 1H), 8.22 (d, J=8.0 Hz, 1H), 8.06 (d, J=6.4 Hz, 2H), 7.56-7.52 (m, 4H), 3.74 (tt, J=8.0, 3.8 Hz, 1H), 3.60-3.54 (m, 2H), 2.06-3.00 (m, 2H), 2.68 (s, 3H), 1.93-1.88 (m, 2H), 1.62-1.58 (m, 2H); MS (m/z): [M] calc'd for C₂₀H₂₂N₄O₃S is 398.14. found 399.10 [M+1].

### Example 58. 1-((2-Methyl-5-(5-phenyl-4H-1,2,4-triazol-3-yl)phenyl)sulfonyl)azetidin-3-ol (SR-32944)

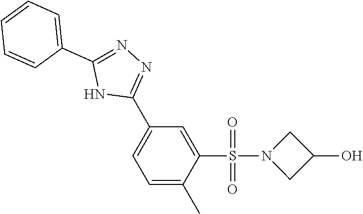 [SMILES: Cc1ccc(-c2nnc(-c3ccccc3)[nH]2)cc1S(=O)(=O)N1CC(O)C1]

This compound was prepared according to the procedure for SR-25604 in 49% overall yield starting with 3-hydroxyazetidine HCl and 5-bromo-2-methylbenzene sulfonyl chloride. ¹H NMR (CD₃OD) δ 8.66 (d, J=1.6 Hz, 1H), 8.20 (dd, J=7.8, 1.4 Hz, 1H), 8.06 (dd, J=6.8, 1.6 Hz, 2H), 7.54-7.52 (m, 4H), 3.66 (quintet, 6.0 Hz, 1H), 3.53-3.44 (m, 2H), 3.10 (dd, J=13.2, 5.2 Hz, 1H), 2.93 (dd, J=13.4, 7.0 Hz, 1H), 2.72 (s, 3H).

### Example 59. 2-Methyl-5-(5-phenyl-4H-1,2,4-triazol-3-yl)-N-(pyridin-2-ylmethyl)benzene sulfonamide (SR-32144)

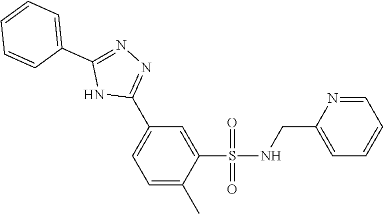 [SMILES: Cc1ccc(-c2nnc(-c3ccccc3)[nH]2)cc1S(=O)(=O)NCc1ccccn1]

This compound was prepared according to the procedure for SR-25604 in 27% overall yield starting with 2-aminomethylpyridine and 5-bromo-2-methylbenzene sulfonyl chloride. ¹H NMR (CD₃OD) δ 8.69 (d, J=5.2 Hz, 1H), 8.56 (d, J=1.6 Hz, 1H), 8.44 (td, J=8.0, 1.6 Hz, 1H), 8.20 (dd, J=8.0, 1.6 Hz, 1H), 8.07-8.03 (m, 2H), 7.85 (t, J=6.4 Hz, 1H), 7.61-7.51 (m, 5H), 4.53 (s, 2H), 2.73 (s, 3H).

### Example 60. 1-((2-Methyl-5-(5-phenyl-4H-1,2,4-triazol-3-yl)phenyl)sulfonyl)pyrrolidin-3-ol (SR-32284)

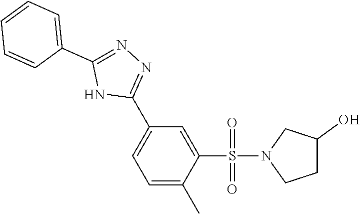 [SMILES: Cc1ccc(-c2nnc(-c3ccccc3)[nH]2)cc1S(=O)(=O)N1CCC(O)C1]

This compound was prepared according to the procedure for SR-25604 in 66% overall yield starting with 3-pyrrolidinol and 5-bromo-2-methylbenzene sulfonyl chloride. ¹H NMR (CD₃OD) δ 8.63 (d, J=2.0 Hz, 1H), 8.20 (dd, J=7.8, 1.8H, 1H), 8.06 (dt, J=5.6, 1.6 Hz, 2H), 7.56-7.50 (m, 4H), 4.45-4.42 (m, 1H), 3.52-3.47 (m, 3H), 3.29-3.28 (m, 1H), 2.70 (s, 3H), 2.12-1.96 (m, 2H); MS (m/z): [M] calc'd for C₁₉H₂₀N₄O₃S is 384.13, found 384.94 [M+1].

### Example 61. 3-Methyl-4-((2-methyl-5-(5-phenyl-4H-1,2,4-triazol-3-yl)phenyl)sulfonyl) morpholine (SR-32286)

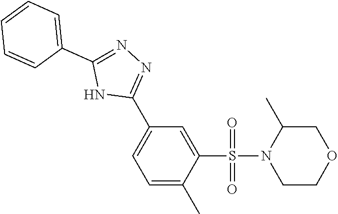 [SMILES: Cc1ccc(-c2nnc(-c3ccccc3)[nH]2)cc1S(=O)(=O)N1CCOCC1C]

This compound was prepared according to the procedure for SR-25604 in 48% overall yield starting with 3-methylmorpholine and 5-bromo-2-methylbenzene sulfonyl chloride. ¹H NMR (CDCl₃) δ 8.72 (d, J=1.2 Hz, 1H), 8.22 (dd, J=8.0, 1.6H, 1H), 8.04-8.01 (m, 2H), 7.47-7.45 (m, 3H), 7.40 (d, J=8.0 Hz, 1H), 3.92 (q, J=6.8 Hz, 1H), 3.80 (dd, J=9.2, 1.6 Hz, 1H), 3.67 (qd, J=7.8, 2.6 Hz, 1H), 3.50-3.35 (m, 2H), 3.28 (d, J=13.2 Hz, 1H), 2.64 (s, 3H), 1.34 (d, J=6.8 Hz, 3H); MS (m/z): [M] calc'd for C₂₀H₂₂N₄O₃S is 398.14. found 398.7 [M+1].

### Example 62. 1-((2-Methyl-5-(5-phenyl-4H-1,2,4-triazol-3-yl)phenyl)sulfonyl)-4-(oxetan-3-yl)piperazine (SR-32324)

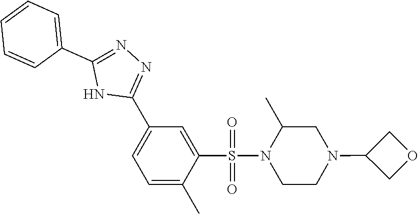 [SMILES: Cc1ccc(-c2nnc(-c3ccccc3)[nH]2)cc1S(=O)(=O)N1CCN(C2COC2)CC1C]

This compound was prepared according to the procedure for SR-25604 in 34% overall yield starting with 1-(oxetan-3-yl)piperazine and 5-bromo-2-methylbenzene sulfonyl chloride. ¹H NMR (CDCl₃) δ 8.58 (s, 1H), 8.26 (d, J=8.0 Hz, 1H), 8.04-8.12 (m, 2H), 7.51-7.48 (m, 3H), 7.44 (d, J=8.0 Hz, 1H), 4.65 (qd, 6.4 Hz, 3.2 Hz, 4H), 3.65 (quintet, J=6.4 Hz, 1H), 3.41 (m, 4H), 2.67 (s, 3H), 2.57 (m, 4H); MS (m/z): [M] calc'd for C₂₂H₂₅N₅O₃S is 439.17. found 440.3 [M+1].

### Example 63. (3aR,6aS)-5-((2-Methyl-5-(5-phenyl-4H-1,2,4-triazol-3-yl)phenyl)sulfonyl) hexahydro-1H-furo[3,4-c]pyrrole (SR-32404)

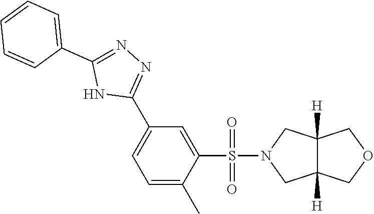 [SMILES: Cc1ccc(-c2nnc(-c3ccccc3)[nH]2)cc1S(=O)(=O)N1C[C@H]2COC[C@H]2C1]

This compound was prepared according to the procedure for SR-25604 in 72% overall yield starting with (3aR,6aS)-hexahydro-1H-furo[3,4-c]pyrrole and 5-bromo-2-methylbenzene sulfonyl chloride. ¹H NMR (CDCl₃) δ 8.54 (d, J=1.2 Hz, 1H), 8.27 (dd, J=8.0, 1.6 Hz, 1H), 8.09-8.07 (m, 2H), 7.47-7.42 (m, 4H), 3.88 (dd, J=6.4, 2.8 Hz, 2H), 3.75 (dd, J=10.6, 2.6 Hz, 2H), 3.54 (dd, J=7.2, 3.2 Hz, 2H); 3.24 (dd, J=10.4, 2.4 Hz, 2H), 3.00 (q, J=3.2 Hz, 2H), 2.69 (s, 3H); MS (m/z): [M] calc'd for C₂₁H₂₂N₄O₃S is 410.14. found 410.6 [M+1].

### Example 64. 8-((2-Methyl-5-(5-phenyl-4H-1,2,4-triazol-3-yl)phenyl)sulfonyl)-3-oxa-8-azabicyclo[3.2.1]octane (SR-32704)

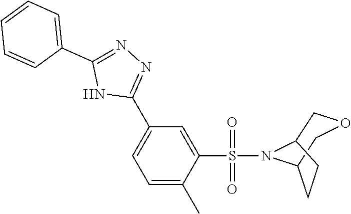 [SMILES: Cc1ccc(-c2nnc(-c3ccccc3)[nH]2)cc1S(=O)(=O)N1C2CCC1COC2]

This compound was prepared according to the procedure for SR-25604 in 48% overall yield starting with 3-oxa-8-azabicyclo[3.2.1]octane and 5-bromo-2-methylbenzene sulfonyl chloride. ¹H NMR (CDCl₃) δ 8.74 (d, J=1.2 Hz, 1H), 8.23 (dd, J=7.6, 1.6 Hz, 1H), 8.03-8.01 (m, 2H), 7.47-7.42 (m, 3H), 7.41 (d, J=8.0 Hz, 1H), 4.03 (s, 2H), 3.67 (d, J=10.8 Hz, 2H), 3.57 (d, J=10.0 Hz, 2H), 2.73 (s, 3H), 2.08-1.99 (m, 4H); MS (m/z): [M] calc'd for C₂₁H₂₂N₄O₃S is 410.14. found 410.9 [M+1].

### Example 65. 8-((2-Methyl-5-(5-phenyl-4H-1,2,4-triazol-3-yl)phenyl)sulfonyl)-1,4-dioxa-8-azaspiro[4.5]decane (SR-32705)

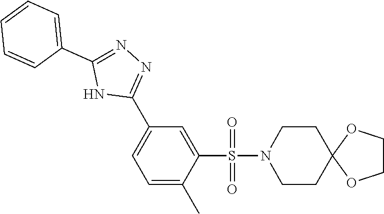 [SMILES: Cc1ccc(-c2nnc(-c3ccccc3)[nH]2)cc1S(=O)(=O)N1CCC2(CC1)OCCO2]

This compound was prepared according to the procedure for SR-25604 in 65% overall yield starting with 1,4-dioxa-8-azaspiro[4.5]decane and 5-bromo-2-methylbenzene sulfonyl chloride. ¹H NMR (CDCl₃) δ 8.61 (s, 1H), 8.18 (d, J=8.0 Hz, 1H), 8.03-8.01 (m, 2H), 7.44-7.42 (m, 3H), 7.36 (d, J=8.0 Hz, 1H), 3.90 (s, 4H), 3.33 (t, J=5.6 Hz, 4H), 2.64 (s, 3H), 1.75 (t, J=5.6 Hz, 4H); MS (m/z): [M] calc'd for C₂₂H₂₄N₄O₄S is 440.15, found 440.9 [M+1].

### Example 66. 9-((2-Methyl-5-(5-phenyl-4H-1,2,4-triazol-3-yl)phenyl)sulfonyl)-1,5-dioxa-9-azaspiro[5.5]undecane (SR-32744)

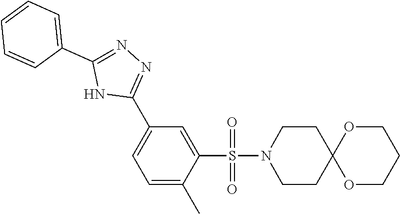 [SMILES: Cc1ccc(-c2nnc(-c3ccccc3)[nH]2)cc1S(=O)(=O)N1CCC2(CC1)OCCCO2]

This compound was prepared according to the procedure for SR-25604 in 69% overall yield starting with piperidone-4-propyleneketal and 5-bromo-2-methylbenzene sulfonyl chloride. ¹H NMR (CDCl₃) δ 8.63 (d, J=1.2 Hz, 1H), 8.21 (dd, J=7.6, 1.6 Hz, 1H), 8.05-8.02 (m, 2H), 7.47-7.40 (m, 3H), 7.39 (d, J=8.0 Hz, 1H), 3.83 (t, J=5.4 Hz, 4H), 3.27 (t, J=5.4 Hz, 4H), 2.65 (s, 3H), 1.93 (t, J=5.6 Hz, 4H), 1.68 (quintet, J=5.6 Hz, 2H); MS (m/z): [M] calc'd for C₂₃H₂₆N₄O₄S is 454.17. found 455.1 [M+1].

### Example 67. 2-Methyl-5-(5-phenyl-4H-1,2,4-triazol-3-yl)-N-(1,4-dioxaspiro[4.5]decan-8-yl)benzenesulfonamide (SR-32764)

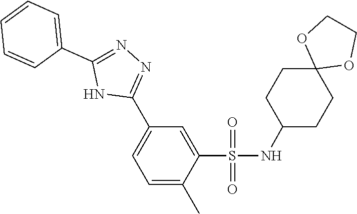 [SMILES: Cc1ccc(-c2nnc(-c3ccccc3)[nH]2)cc1S(=O)(=O)NC1CCC2(CC1)OCCO2]

This compound was prepared according to the procedure for SR-25604 in 51% overall yield starting with 1,4-dioxaspiro[4,5] dec-8-yl amine and 5-bromo-2-methylbenzene sulfonyl chloride. ¹H NMR (CD₃OD) δ 8.70 (d, J=1.2 Hz, 1H), 8.18 (d, J=8.0 Hz, 1H), 8.06 (m, 3H), 7.52 (m, 4H), 3.90-3.83 (m, 4H), 3.19 (tt, J=8.0, 4.0 Hz, 1H), 2.71 (s, 3H), 1.75-1.55 (m, 8H); MS (m/z): [M] calc'd for C₂₃H₂₆N₄O₄S is 454.17. found 454.7 [M+1].

### Example 68. 1-((2-Methyl-5-(5-phenyl-4H-1,2,4-triazol-3-yl)phenyl)sulfonyl)-4-phenyl piperidin-4-ol (SR-32784)

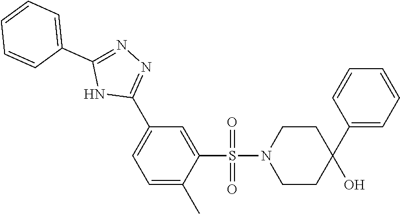 [SMILES: Cc1ccc(-c2nnc(-c3ccccc3)[nH]2)cc1S(=O)(=O)N1CCC(O)(c2ccccc2)CC1]

This compound was prepared according to the procedure for SR-25604 in 51% overall yield starting with 4-hydroxy-4-phenylpiperidine and 5-bromo-2-methylbenzene sulfonyl chloride. ¹H NMR (CD₃OD) δ 8.66 (d, J=1.2 Hz, 1H), 8.24 (d, J=8.0 Hz, 1H), 8.06 (m, 3H), 7.53 (m, 4H), 7.46 (d, J=8.4 Hz, 2H), 7.32 (t, J=7.6 Hz, 2H), 7.22 (td, J=7.w, 1.2 Hz, 1H), 3.73 (d, J=12.0 Hz, 2H), 3.21 (t, J=12.0 Hz, 2H), 2.73 (s, 3H), 2.17-2.08 (m, 2H), 1.79 (d, J=13.2 Hz, 2H).

### Example 69. 1-((2-Methyl-5-(5-phenyl-4H-1,2,4-triazol-3-yl)phenyl)sulfonyl)-4-(pyridin-3-yl)piperidin-4-ol (SR-32924)

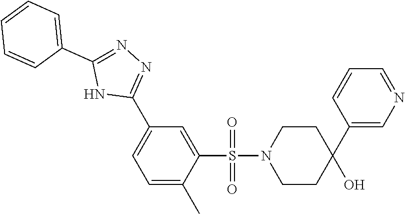 [SMILES: Cc1ccc(-c2nnc(-c3ccccc3)[nH]2)cc1S(=O)(=O)N1CCC(O)(c2cccnc2)CC1]

This compound was prepared according to the procedure for SR-25604 in 63% overall yield starting with 4-(pyridin-3-yl)piperidin-4-ol and 5-bromo-2-methylbenzene sulfonyl chloride. ¹H NMR (CD₃OD) δ 8.84 (s, 1H), 8.66 (d, J=1.6 Hz, 1H), 8.60 (s, 1H), 8.36 (d, J=8.4 Hz, 1H), 8.25 (dd, J=8.0, 1.6 Hz, 1H), 8.08-8.05 (m, 2H), 7.75 (dd, J=5.4, 2.4 Hz, 1H), 7.60-7.53 (m, 4H), 3.80 (dd, J=11.6, 2.4 Hz, 2H), 3.23 (td, J=12.0, 2.0 Hz, 2H), 2.74 (s, 3H), 2.20 (td, J=13.0, 4.4 Hz, 2H), 1.84 (d, J=13.2 Hz, 2H).

### Example 70. 8-((2-Methyl-5-(5-phenyl-4H-1,2,4-triazol-3-yl)phenyl)sulfonyl)-1-oxa-8-azaspiro[4.5]decane (SR-32925)

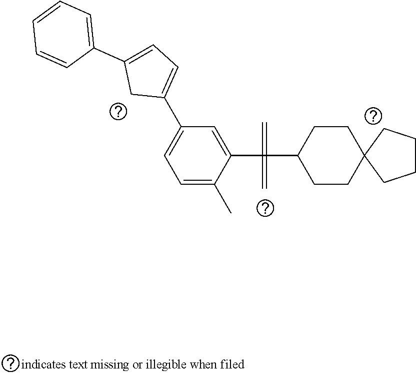

This compound was prepared according to the procedure for SR-25604 in 62% overall yield starting with 1-oxa-8-azaspiro[4.5]decane HCl and 5-bromo-2-methylbenzene sulfonyl chloride. ¹H NMR (CDCl₃) δ 8.58 (s, 1H), 8.15 (d, J=7.6 Hz, 1H), 8.03-8.00 (m, 2H), 7.42-7.41 (m, 3H), 7.32 (d, J=8.0 Hz, 1H), 3.75 (t, J=6.8 Hz, 2H), 3.49-3.46 (m, 2H), 3.08 (td, J=10.8, 4.0 Hz, 2H), 2.62 (s, 3H), 1.87 (quintet, J=6.8 Hz, 2H), 1.67-1.64 (m, 6H); MS (m/z): [M] calc'd for C₂₃H₂₆N₄O₃S is 438.17. found 438.7 [M+1].

### Example 71. (4-((2-Methyl-5-(5-phenyl-4H-1,2,4-triazol-3-yl)phenyl)sulfonyl)morpholin-3-yl)methanol (SR-32945)

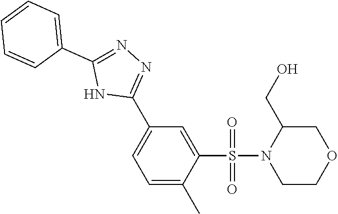 [SMILES: Cc1ccc(-c2nnc(-c3ccccc3)[nH]2)cc1S(=O)(=O)N1CCOCC1CO]

This compound was prepared according to the procedure for SR-25604 in 50% overall yield starting with 3-hydroxymethylmorpholine and 5-bromo-2-methylbenzene sulfonyl chloride. ¹H NMR (CD₃OD) δ 8.72 (s, 1H), 8.22 (d, J=7.6 Hz, 1H), 8.06 (d, J=6.4 Hz, 2H), 7.56-7.52 (m, 4H), 4.07 (d, J=12.0 Hz, 1H), 3.98-3.92 (m, 1H), 3.81 (dd, J=11.2, 2.4 Hz, 1H), 3.72-3.68 (m, 2H), 3.59 (dd, J=12.0, 2.4 Hz, 1H), 3.49-3.33 (m, 3H), 2.68 (s, 3H); MS (m/z): [M] calc'd for C₂₀H₂₂N₄O₄S is 414.14. found 414.6 [M+1].

### Example 72. 1′-((2-Methyl-5-(5-phenyl-4H-1,2,4-triazol-3-yl)phenyl)sulfonyl)-3H-spiro[isobenzofuran-1,4′-piperidine] (SR-32984)

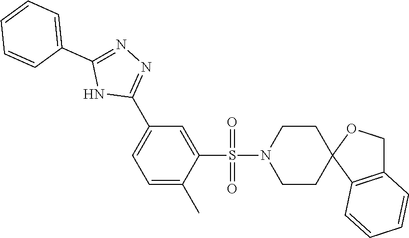 [SMILES: Cc1ccc(-c2nnc(-c3ccccc3)[nH]2)cc1S(=O)(=O)N1CCC2(CC1)OCc1ccccc12]

This compound was prepared according to the procedure for SR-25604 in 56% overall yield starting with 3H-spiro[isobenzofuran-1,4′-piperidine] HCl and 5-bromo-2-methylbenzene sulfonyl chloride. ¹H NMR (CDCl₃) δ 8.64 (s, 1H), 8.22 (d, J=7.6 Hz 1H), 8.05-8.03 (m, 2H), 7.45-7.44 (m, 3H), 7.40 (d, J=8.0 Hz, 1H), 7.29-7.24 (m, 3H), 7.18 (dd, J=6.8, 2.0 Hz, 1H), 7.08 (d, J=6.8, 1.6 Hz, 1H), 5.00 (s, 2H), 3.83 (d, J=11.2 Hz, 2H), 3.10 (t, J=11.6 Hz, 2H), 2.70 (s, 3H), 2.02 (td, J=13.0, 5.2 Hz, 2H), 1.77 (d, J=13.2 Hz, 2H); MS (m/z): [M] calc'd for C₂₇H₂₆N₄O₃S is 486.17. found 487.0 [M+1].

### Example 73. 4-Benzyl-1-((2-methyl-5-(5-phenyl-4H-1,2,4-triazol-3-yl)phenyl)sulfonyl) piperidin-4-ol (SR-33024)

This compound was prepared according to the procedure for SR-25604 in 47% overall yield starting with 4-benzylpiperidin-4-ol and 5-bromo-2-methylbenzene sulfonyl chloride. ¹H NMR (CDCl₃) δ 8.55 (s, 1H), 8.22 (dd, J=7.6, 1.2 Hz 1H), 8.01-7.99 (m, 2H), 7.42 (t, J=3.2 Hz, 3H), 7.35 (d, J=8.0 Hz, 1H), 7.31-7.14 (m, 3H), 7.12 (d, J=6.4 Hz, 2H), 3.57 (d, J=12.4 Hz, 2H), 2.97 (t, J=11.2 Hz, 2H), 2.72 (s, 2H), 2.62 (s, 3H), 1.72 (td, J=13.0, 4.2 Hz, 2H), 1.52 (d, J=13.2 Hz, 2H); MS (m/z): [M] calc'd for C₂₇H₂₅N₄O₃S is 488.19. found 489.1 [M+1].

### Example 74. 7-((2-Methyl-5-(5-phenyl-4H-1,2,4-triazol-3-yl)phenyl)sulfonyl)-1,4-dioxa-7-azaspiro[4.5]decane (SR-33045)

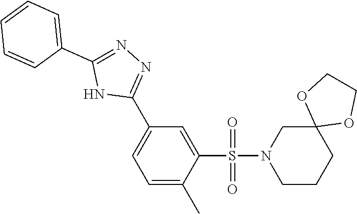 [SMILES: Cc1ccc(-c2nnc(-c3ccccc3)[nH]2)cc1S(=O)(=O)N1CCCC2(C1)OCCO2]

This compound was prepared according to the procedure for SR-25604 in 59% overall yield starting with 1,4-dioxa-7-azaspiro[4.5]decane and 5-bromo-2-methylbenzene sulfonyl chloride. ¹H NMR (CDCl₃) δ 8.69 (d, J=1.6 Hz, 1H), 8.22 (dd, J=7.6, 1.6 Hz, 1H), 8.06-8.04 (m, 2H), 7.46-7.44 (m, 3H), 7.40 (d, J=8.0 Hz, 1H), 3.95-3.84 (m, 4H), 3.19 (s, 4H), 2.68 (s, 3H), 1.77-1.70 (m, 4H); MS (m/z): [M] calc'd for C₂₂H₂₄N₄O₄S is 440.15. found 441.1 [M+1].

### Example 75. 2-Methyl-5-(5-phenyl-4H-1,2,4-triazol-3-yl)-N-(pyrimidin-5-ylmethyl)benzene sulfonamide (SR-33344)

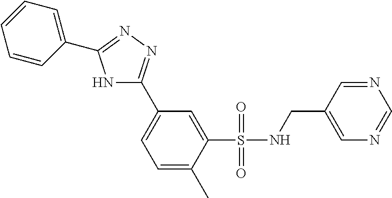 [SMILES: Cc1ccc(-c2nnc(-c3ccccc3)[nH]2)cc1S(=O)(=O)NCc1cncnc1]

This compound was prepared according to the procedure for SR-25604 in 42% overall yield starting with 5-pyrimidinemethanamine and 5-bromo-2-methylbenzene sulfonyl chloride. ¹H NMR (CD₃OD) δ 8.95 (s, 1H), 8.66 (s, 2H), 8.62 (d, J=1.6 Hz, 1H), 8.17 (dd, J=8.0, 1.6 Hz, 1H), 8.07 (dd, J=7.4, 1.4 Hz, 2H), 7.55-7.53 (m, 3H), 7.49 (d, J=8.0 Hz, 1H), 4.26 (s, 2H), 2.68 (s, 3H); MS (m/z): [M] calc'd for C₂₀H₁₅N₆O₂S is 406.12. found 406.6 [M+1].

### Example 76. 1-((2-Methyl-5-(5-phenyl-4H-1,2,4-triazol-3-yl)phenyl)sulfonyl)-4-(2-(pyridin-4-yl)ethyl)piperazine (SR-33364)

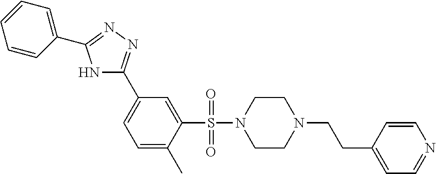 [SMILES: Cc1ccc(-c2nnc(-c3ccccc3)[nH]2)cc1S(=O)(=O)N1CCN(CCc2ccncc2)CC1]

This compound was prepared according to the procedure for SR-25604 in 32% overall yield starting with 1-(2-pyridin-4-yl)ethylpiperazine and 5-bromo-2-methylbenzene sulfonyl chloride. ¹H NMR (CD₃OD) δ 8.64 (d, J=1.6 Hz, 1H), 8.58 (s, 1H), 8.27 (dd, J=8.0, 1.6 Hz, 1H), 8.07-8.04 (m, 2H), 7.62-7.58 (m, 3H), 7.55-7.53 (m, 3H), 3.48-3.47 (m, 4H), 3.28-3.14 (m, 8H), 2.70 (s, 3H); MS (m/z): [M] calc'd for C₂₆H₂₈N₆O₂S is 488.20, found 488.8 [M+1].

### Example 77. 2-(4-((3-Fluoro-2-methyl-5-(5-phenyl-4H-1,2,4-triazol-3-yl)phenyl)sulfonyl) piperazin-1-yl)ethan-1-ol (SR-33604)

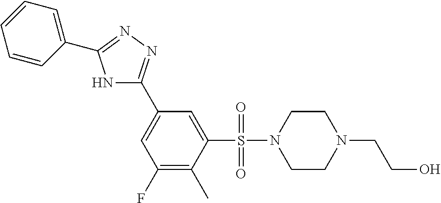 [SMILES: Cc1c(F)cc(-c2nnc(-c3ccccc3)[nH]2)cc1S(=O)(=O)N1CCN(CCO)CC1]

A mixture of 3-fluoro-2-methylbenzene sulfonyl chloride (100 mg, 0.48 mmol), NaHCO₃ (403 mg, 4.8 mmol), and N-(2-hydroxyethyl)piperazine (71 μL, 0.58 mmol) in DCM was stirred overnight at room temperature. Upon completion, the solvent was removed under reduced pressure and purification via column chromatography afforded product 2-(4-((3-fluoro-2-methylphenyl)sulfonyl)piperazin-1-yl)ethan-1-ol (164 mg). MS (m/z): [M] calc'd for C₁₃H₁₉FN₂O₃S is 302.11. found 303.0 [M+1].

Then, a mixture of the product (164 mg) and tribromoisocyanuric acid (66 mg, 0.18 mmol) in TFA (270 μL) stirred overnight at room temperature and then was poured into ice. The aqueous layer was extracted with DCM. The combined organic layers were dried over Na₂SO₄ and concentrated to dryness to afford the crude product 2-(4-((5-bromo-3-fluoro-2-methylphenyl)sulfonyl)piperazin-1-yl)ethan-1-ol, which was used without further purification. MS (m/z): [M] calc'd for C₁₃H₁₈BrFN₂O₃S is 382.02/380.02. found 382.9/380.9 [M+1].

Then, a mixture of the crude product, dppf (11 mg, 0.02 mmol), de-ionized water (24 μL), and Zn(CN)₂ (45 mg, 0.38 mmol) in DMF (2.4 mL) was de-gassed and back-filled with argon 3 times at room temperature in a microwave vial. Pd₂(dba)₃ (7.8 mg, 0.008 mmol) was added, the vial was sealed, and the mixture was de-gassed and back-filled with argon. Then, the reaction mixture was microwaved in a Biotage© microwave reactor at 115° C. for 30 min under normal absorption conditions. Upon completion, solvent was removed under reduced pressure and the crude product 3-fluoro-5-((4-(2-hydroxyethyl)piperazin-1-yl)sulfonyl)-4-methylbenzonitrile was used without purification. MS (m/z): [M] calc'd for C₁₄H₁₈FN₃O₃S is 327.11. found 327.8.

Finally, a mixture of the crude product, K₂CO₃ (68 mg, 0.49 mmol), and benzhydrazide (68 mg, 0.49 mmol) was stirred at 150° C. in a pre-heated oil bath for 4 hours. Purification via prep HPLC followed by column chromatography afforded pure product SR-33604 (13 mg, 6.1% yield over 4 steps). ¹H NMR (CD₃OD) δ 8.48 (s, 1H), 8.09-8.04 (m, 3H), 7.58-7.52 (m, 3H), 3.70 (t, J=5.6 Hz, 2H), 3.36 (t, J=5.0 Hz, 4H), 2.83 (t, J=4.4 Hz, 4H), 2.74 (t, J=5.6 Hz, 2H), 2.59 (d, J=2.4 Hz, 3H); MS (m/z): [M] calc'd for C₂₁H₂₄FN₅O₃S is 445.16. found 445.8 [M+1].

### Example 78. 2-(1-((2-Methyl-5-(5-phenyl-4H-1,2,4-triazol-3-yl)phenyl)sulfonyl)piperidin-4-yl)ethan-1-ol (SR-33725)

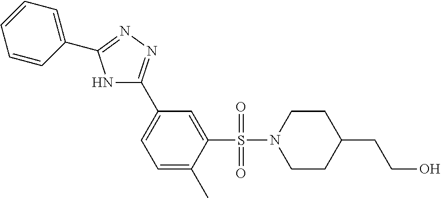 [SMILES: Cc1ccc(-c2nnc(-c3ccccc3)[nH]2)cc1S(=O)(=O)N1CCC(CCO)CC1]

This compound was prepared according to the procedure for SR-25604 in 49% overall yield starting with 4-piperidine ethanol and 5-bromo-2-methylbenzene sulfonyl chloride. ¹H NMR (CD₃OD) δ 8.60 (s, 1H) 8.22 (d, J=7.6 Hz, 1H), 8.07-8.05 (m, 2H), 7.56-7.52 (m, 4H), 3.78 (d, J=12.4 Hz, 2H), 3.59 (t, J=6.6 Hz, 2H), 2.72 (d, J=12.0 Hz, 2H), 2.68 (s, 3H), 1.80 (d, J=12.0 Hz, 3H), 1.56-1.45 (m, 3H), 1.31-1.24 (m, 2H); MS (m/z): [M] calc'd for C₂₂H₂₆N₄O₃S is 426.17. found 427.0 [M+1].

### Example 79. 1-(2-Methoxyethyl)-4-((2-methyl-5-(5-phenyl-4H-1,2,4-triazol-3-yl)phenyl) sulfonyl)piperazine (SR-34533)

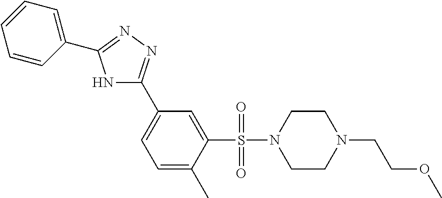 [SMILES: COCCN1CCN(S(=O)(=O)c2cc(-c3nnc(-c4ccccc4)[nH]3)ccc2C)CC1]

A solution of 3-((4-(2-Hydroxyethyl)piperazin-1-yl)sulfonyl-4-methylbenzonitrile (89 mg, 0.29 mmol) in THF was cooled in an ice-water bath under argon. NaH (14 mg, 0.35 mmol) was added and the reaction mixture was stirred for 30 min in the ice-water bath. MeI (21 μL, 0.34 mmol) was added and the reaction mixture was warmed slowly to room temperature overnight. Upon completion, solvent was removed under reduced pressure and purification via column chromatography afforded pure product 3-((4-(2-methoxyethyl)piperazin-1-yl)sulfonyl)-4-methylbenzonitrile (29 mg, 31% yield). MS (m/z): [M] calc'd for C₁₅H₂₁N₃O₃S is 323.13. found 324.0 [M+1].

Then, a mixture of the product (29 mg, 0.09 mmol), K₂CO₃ (12 mg, 0.09 mmol), and benzhydrazide (24 mg, 0.18 mmol) in 1-BuOH (90 μL) was stirred at 150° C. in a pre-heated oil bath for 24 hours. Upon completion, solvent was removed under reduced pressure and purification via prep HPLC followed by column chromatography afforded pure product SR-34533 (12 mg, 30% yield). ¹H NMR (CD₃OD) δ 8.64 (d, J=1.6 Hz, 1H), 8.24 (dd, J=8.0, 1.6 Hz, 1H), 8.06 (dd, J=7.2, 1.8 Hz, 2H), 7.57-7.51 (m, 4H), 3.50 (t, J=5.4 Hz, 2H), 3.30 (s, 3H), 3.25 (t, J=4.8 Hz, 4H), 2.69 (s, 3H), 2.61-2.58 (m, 6H); MS (m/z): [M] calc'd for C₂₂H₂₇N₅O₃S is 441.18. found 441.8 [M+1].

### Example 80. tert-Butyl (2-(4-((2-methyl-5-(5-phenyl-4H-1,2,4-triazol-3-yl)phenyl)sulfonyl)piperazin-1-yl)ethyl)carbamate (SR-34024)

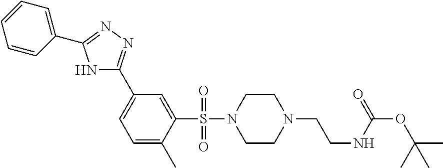 [SMILES: Cc1ccc(-c2nnc(-c3ccccc3)[nH]2)cc1S(=O)(=O)N1CCN(CCNC(=O)OC(C)(C)C)CC1]

This compound was prepared according to the procedure for SR-25604 in 16% overall yield starting with 1-(2N-Boc-aminoethyl)piperazine and 5-bromo-2-methylbenzene sulfonyl chloride. ¹H NMR (CD₃OD) δ 8.60 (s, 1H) 8.22 (d, J=7.6 Hz, 1H), 8.06-8.04 (m, 2H), 7.55-7.52 (m, 4H), 3.23 (m, 4H), 3.14 (t, J=6.2 Hz, 2H), 2.62 (s, 3H), 2.57-2.62 (m, 4H), 2.46 (t, J=6.4 Hz, 2H), 1.38 (s, 9H).

### Example 81. 1-((2-Methyl-5-(2-morpholinopyridin-4-yl)phenyl)sulfonyl)azetidin-3-ol (SR-34464)

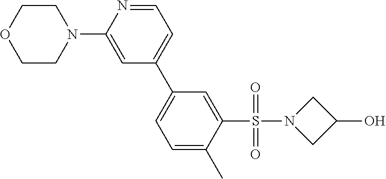 [SMILES: Cc1ccc(-c2ccnc(N3CCOCC3)c2)cc1S(=O)(=O)N1CC(O)C1]

This compound was prepared according to the procedure for SR-29224 in 32% overall yield starting with 3-hydroxyazetidine HCl and 5-bromo-2-methylbenzene sulfonyl chloride. ¹H NMR (CD₃OD) δ 8.24 (d, J=2.0 Hz, 1H), 8.08 (d, J=6.4 Hz, 1H), 7.99 (dd, J=7.6, 2.0 Hz, 1H), 7.60 (d, J=8.0 Hz, 1H), 7.42, (s, 1H), 7.23 (dd, J=6.2, 1.4 Hz, 1H), 4.54-4.48 (m, 1H), 3.99 (td, J=7.8, 2.0, 2H), 3.87 (t, J=5.0 Hz, 4H), 3.75-3.69 (m, 6H), 2.70 (s, 3H); MS (m/z): [M] calc'd for C₁₉H₂₃N₃O₄S is 389.14. found 390.1 [M+H].

### Example 82. 2-(4-((2,4-Dimethyl-5-(5-phenyl-4H-1,2,4-triazol-3-yl)phenyl)sulfonyl)

piperazin-1-yl)ethan-1-ol (SR-34778)

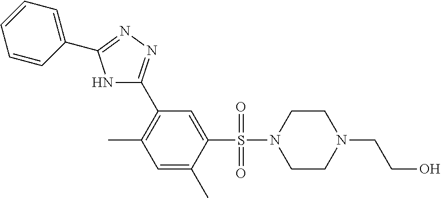 [SMILES: Cc1cc(C)c(S(=O)(=O)N2CCN(CCO)CC2)cc1-c1nnc(-c2ccccc2)[nH]1]

This compound was prepared according to the procedure for SR-25604 in 32% overall yield starting with N-(2-hydroxyethyl)piperazine and 5-bromo-2,4-dimethylbenzene sulfonyl chloride. ¹H NMR (CD₃OD) δ 8.36 (s, 1H), 8.06-8.04 (m, 2H), 7.54-7.53 (m, 3H), 7.46 (s, 1H), 3.86 (t, J=5.2 Hz, 2H), 3.61-3.43 (m, 8H), 3.28 (t, J=5.2 Hz, 2H), 2.66 (s, 3H); MS (m/z): [M] calc'd for C₂₂H₂₇N₅O₃S is 441.18. found 442.2 [M+H].

### Example 83. 2-(4-((5-(2-Chloropyridin-4-yl)-2-methylphenyl)sulfonyl)piperazin-1-yl)ethan-1-ol (SR-34793)

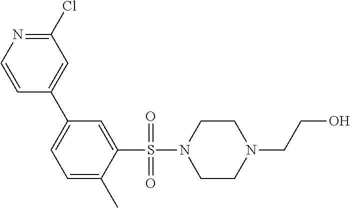 [SMILES: Cc1ccc(-c2ccnc(Cl)c2)cc1S(=O)(=O)N1CCN(CCO)CC1]

A mixture of 5-bromo-2-methylphenyl sulfonyl piperazin-1-yl (71 mg, 0.20 mmol), K₂CO₃ (54 mg, 0.39 mmol), de-ionized water (49 μL), and 2-chloro-pyridine-4-boronic acid (38 mg, 0.24 mmol) in 1,4-dioxane (1.9 mL) was de-gassed and back-filled with argon 3 times at room temperature in a microwave vial. Pd(PPh₃)₄ (11 mg, 0.01 mmol) was added, the vial was sealed, and the mixture was de-gassed and back-filled with argon. Then, the reaction mixture was stirred for 2 hours at 120° C. in a pre-heated oil bath. Upon completion, solvent was removed under reduced pressure and purification via prep HPLC followed by column chromatography afforded pure product SR-34793 (55 mg, 71% yield). ¹H NMR (CDCl₃) δ 8.45 (dd, J=5.2, 0.4 Hz, 1H), 8.24 (d, J=2.0 Hz, 1H), 7.98 (dd, J=7.8, 1.8 Hz, 1H), 7.80 (d, J=1.2 Hz, 1H), 7.70 (dd, J=5.2, 1.6 Hz, 1H) 7.63 (d, J=8.0 Hz, 1H), 3.87 (t, J=5.2 Hz, 2H), 3.70-3.46 (m, 10H), 2.70 (s, 3H); MS (m/z): [M] calc'd for C₁₈H₂₂ClN₃O₃S is 395.11. found 396.1 [M+H].

### Example 84. 2-(4-((5-(2-Chloropyridin-4-yl)-4-fluoro-2-methylphenyl)sulfonyl)piperazin-1-yl)ethan-1-ol (SR-35015)

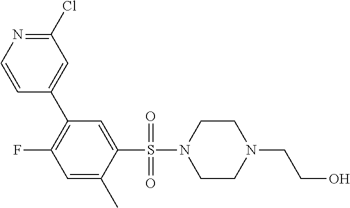 [SMILES: Cc1cc(F)c(-c2ccnc(Cl)c2)cc1S(=O)(=O)N1CCN(CCO)CC1]

This compound was prepared according to the procedure for SR-34793 in 86% overall yield starting with 5-bromo-4-fluoro-2-methylbenzene sulfonyl chloride. ¹H NMR (CD₃OD) δ 8.48 (d, J=5.2 Hz, 1H), 8.14 (d, J=7.6 Hz, 1H), 7.72 (s, 1H), 7.61 (dt, J=5.6, 1.4 Hz, 1H), 7.45 (d, J=11.2 Hz, 1H), 3.87 (t, J=5.2 Hz, 2H), 3.48-3.45 (m, 10H), 2.69 (s, 3H); MS (m/z): [M] calc'd for C₁₈H₂₁ClFN₃O₃S is 413.10. found 414.2 [M+H].

### Example 85. 2-(4-((5-(2-Chloropyridin-4-yl)-2,4-dimethylphenyl)sulfonyl)piperazin-1-yl)ethan-1-ol (SR-34951)

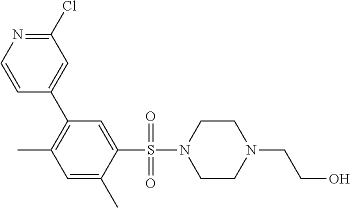 [SMILES: Cc1cc(C)c(S(=O)(=O)N2CCN(CCO)CC2)cc1-c1ccnc(Cl)c1]

This compound was prepared according to the procedure for SR-34793 in 53% overall yield starting with 5-bromo-2,4-dimethylbenzene sulfonyl chloride. ¹H NMR (CD₃OD) δ 8.45 (d, J=5.2 Hz, 1H), 7.75 (s, 1H), 7.51 (s, 1H), 7.46 (s, 1H), 7.41 (dd, J=4.6 Hz, 1.2 Hz, 1H), 3.88 (t, J=5.0 Hz, 2H), 3.46 (m, 10H), 2.65 (s, 3H), 2.34 (s, 3H); MS (m/z): [M] calc'd for C₁₉H₂₄ClN₃O₃S is 409.12. found 409.88 [M+H].

### Example 86. 1-((5-(2-Chloropyridin-4-yl)-2-methylphenyl)sulfonyl)-4-(2-fluoroethyl) piperazine (SR-35017)

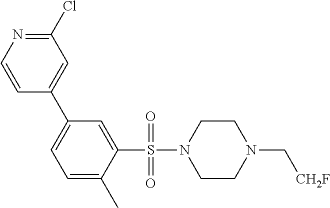

This compound was prepared according to the procedure for SR-34793 in 27% overall yield starting with 1-(2-fluoroethyl)piperazine HCl. ¹H NMR (CD₃OD) δ 8.44 (d, J=5.2 Hz, 1H), 8.18 (d, J=1.6 Hz, 1H), 7.94 (dd, J=7.8, 1.8 Hz, 1H), 7.78 (s, 1H), 7.68 (dd, J=5.2, 1.6 Hz, 1H), 7.59 (d, J=8.0 Hz, 1H), 4.60 (t, J=4.8 Hz, 1H), 4.48 (t, J=4.6 Hz, 1H), 3.24 (t, J=4.6 Hz, 4H), 2.75 (t, J=4.6 Hz, 1H), 2.69-2.67 (m, 4H), 2.62 (t, J=4.6 Hz, 4H); MS (m/z): [M] calc'd for C₁₈H₂₁ClFN₃O₂S is 397.10. found 397.88 [M+H].

### Example 87. 3-(4-((5-(2-Chloropyridin-4-yl)-2-methylphenyl)sulfonyl)piperazin-1-yl)propan-1-ol (SR-35129)

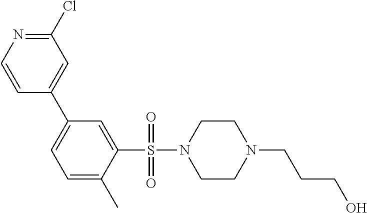 [SMILES: Cc1ccc(-c2ccnc(Cl)c2)cc1S(=O)(=O)N1CCN(CCCO)CC1]

This compound was prepared according to the procedure for SR-34793 in 24% overall yield starting with N-(3-hydroxypropyl)piperazine. ¹H NMR (CD₃OD) δ 8.44 (d, J=5.2 Hz, 1H), 8.18 (s, 1H), 7.94 (d, J=8.0 Hz, 1H), 7.78 (s, 1H), 7.68 (dd, J=5.2, 1.6 Hz, 1H), 7.59 (d, J=8.0 Hz, 1H), 3.59 (t, J=6.2 Hz, 2H), 3.23 (m, 4H), 2.69 (s, 3H), 2.57 (m, 4H), 2.50 (t, J=7.2 Hz, 2H), 1.70 (quintet, J=6.8 Hz, 2H); MS (m/z): [M] calc'd for C₁₉H₂₄ClN₃O₃S is 409.12. found 410.1 [M+H].

### Example 88. 1-((5-(2-Chloropyridin-4-yl)-2-methylphenyl)sulfonyl)-4-(2,2-difluoroethyl) piperazine (SR-35124)

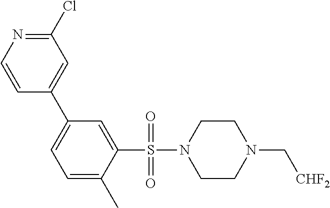 [SMILES: Cc1ccc(-c2ccnc(Cl)c2)cc1S(=O)(=O)N1CCN(CC(F)F)CC1]

This compound was prepared according to the procedure for SR-34793 in 24% overall yield starting with 1-(2,2-difluoroethyl)piperazine. ¹H NMR ((CD₃)₂CO) δ 8.49 (d, J=5.2 Hz, 1H), 8.21 (d, J=2.0 Hz, 1H), 8.02 (dd, J=8.0, 2.0 Hz, 1H), 7.80 (d, J=0.8 Hz, 1H), 7.73 (dd, J=5.2, 1.6 Hz, 1H), 7.63 (d, J=8.0 Hz, 1H), 6.02 (tt, J=55.8, 4.2 Hz, 1H), 3.24 (t, J=5.0 Hz, 4H), 2.86 (td, J=15.2, 4.4 Hz, 2H), 2.74 (t, J=4.8 Hz, 4H), 2.70 (s, 3H); MS (m/z): [M] calc'd for C₁₈H₂₀ClF₂N₃O₂S is 415.09. found 416.2 [M+H].

### Example 89. 1-(4-(4-Methyl-3-((4-(2-(pyridin-4-yl)ethyl)piperazin-1-yl)sulfonyl)phenyl)-1H-pyrazol-1-yl)hept-6-yn-3-one (SR-35186)

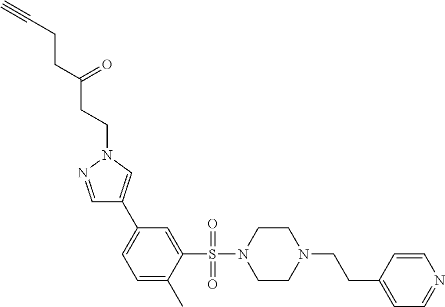 [SMILES: C#CCCC(=O)CCn1cc(-c2ccc(C)c(S(=O)(=O)N3CCN(CCc4ccncc4)CC3)c2)cn1]

A mixture of 1-hydroxyhept-6-yn-3-one (31 mg, 0.25 mmol) and Et₃N (41 μL, 0.3 mmol) in DCM was cooled in an ice-water bath. Methane sulfonyl chloride (29 μL, 0.37 mmol) was added and the reaction mixture was warmed slowly to room temperature overnight. Upon completion, the solvent was removed under reduced pressure and the residue was re-suspended in ACN (972 μL). 1-((2-Methyl-5-(1H-pyrazol-4-yl)phenyl)sulfonyl)-4-(2-pyridin-4-yl)ethyl)piperazine (40 mg, 0.10 mmol) and Cs₂CO₃ (38 mg, 0.12 mmol) were added, and then the reaction mixture was stirred overnight at 90° C. in a pre-heated oil bath. Upon completion, the solvent was removed under reduced pressure and purification via prep HPLC followed by column chromatography afforded pure product SR-35186 (7.1 mg, 14% yield). ¹H NMR (CD₃OD) δ 8.70 (br, 2H), 8.04 (s, 1H), 7.98 (d, J=2.0 Hz, 1H), 7.88 (s, 1H), 7.83 (br, 2H), 7.71 (dd, J=8.0, 1.6 Hz, 7.42 (d, J=8.4 Hz, 1H), 4.61 (t, J=6.6 Hz, 2H), 3.58 (t, J=7.2 Hz, 2H), 3.55-3.36 (m, 10H), 3.03 (t, J=6.8 Hz, 2H), 2.74 (t, J=7.2 Hz, 2H), 2.60 (s, 3H), 2.44-2.40 (m, 2H), 2.24 (t, J=2.6 Hz, 1H); MS (m/z): [M] calc'd for C₂₈H₃₃N₅O₃S is 519.23. found 520.2 [M+H].

### Example 90. 1-((5-(2-Chloropyridin-4-yl)-2-methylphenyl)sulfonyl)-4-(2,2,2-trifluoroethyl) piperazine (SR-35324)

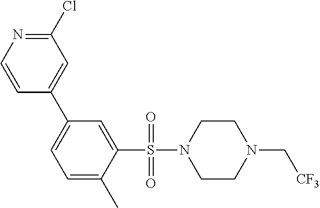 [SMILES: Cc1ccc(-c2ccnc(Cl)c2)cc1S(=O)(=O)N1CCN(CC(F)(F)F)CC1]

This compound was prepared according to the procedure for SR-34793 in 51% overall yield starting with 1-(2,2,2-trifluoroethyl)piperazine dihydrochloride. ¹H NMR ((CD₃)₂CO) δ 8.48 (d, J=5.2 Hz, 1H), 8.21 (d, J=2.0 Hz, 1H), 8.02 (dd, J=8.0, 2.0 Hz, 1H), 7.80 (t, J=0.8 Hz, 1H), 7.73 (dd, J=5.0, 1.4 Hz, 1H), 7.63 (d, J=8.0 Hz, 1H), 3.24-3.14 (m, 6H), 2.79 (t, J=4.8 Hz, 4H), 2.70 (s, 3H); MS (m/z): [M] calc'd for C₁₈H₁₉ClF₃N₃O₂S is 433.08. found 434.1 [M+H].

### Example 91. 1-((5-(2-Chloropyridin-4-yl)-2-methylphenyl)sulfonyl)piperazine (SR-35422)

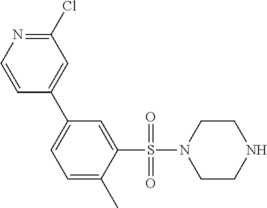 [SMILES: Cc1ccc(-c2ccnc(Cl)c2)cc1S(=O)(=O)N1CCNCC1]

This compound was prepared according to the procedure for SR-34793 in 68% overall yield starting with 1-piperazine carboxaldehyde. ¹H NMR (CD₃OD) δ 8.44 (d, J=5.2 Hz, 1H), 8.24 (d, J=2.0 Hz, 1H), 7.98 (dd, J=8.0, 2.0 Hz, 1H), 7.80 (t, J=1.2 Hz, 1H), 7.70 (dd, J=5.2, 1.6 Hz, 1H), 7.62 (d, J=8.4 Hz, 1H), 3.51 (t, J=5.2 Hz, 4H), 3.33 (m, 4H), 2.69 (s, 3H); MS (m/z): [M] calc'd for C₁₆H₁₈ClN₃O₂S is 351.08. found 352.3 [M+H].

### Example 92. 1-(4-((5-(2-Chloropyridin-4-yl)-2-methylphenyl)sulfonyl)piperazin-1-yl)propan-2-ol (SR-35464)

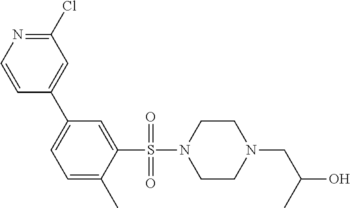 [SMILES: Cc1ccc(-c2ccnc(Cl)c2)cc1S(=O)(=O)N1CCN(CC(C)O)CC1]

A mixture of SR-35422 (27 mg, 0.08 mmol), K₂CO₃ (21 mg, 0.15 mmol), and 1-bromo-2-propanol (10 μL, 0.11 mmol) in ACN was stirred for 3 days at 80° C. in a pre-heated oil bath. Upon completion, the solvent was removed under reduced pressure and purification via column chromatography followed by prep HPLC afforded pure product SR-35464 (8 mg, 25% yield). ¹H NMR (CD₃OD) δ 8.45 (d, J=5.2 Hz, 1H), 8.25 (d, J=2.0 Hz, 1H), 7.99 (dd, J=8.0, 2.0 Hz, 1H), 7.81 (d, J=0.8 Hz, 1H), 7.70 (dd, J=5.2, 1.6 Hz, 1H), 7.62 (d, J=8.0 Hz, 1H), 4.19-4.14 (m, 1H), 3.90-3.50 (m, 8H), 3.21 (dd, J=13.0, 2.6 Hz, 1H), 3.08 (t, J=11.8 Hz, 1H), 2.70 (s, 3H), 1.22 (d, J=6.4 Hz, 3H); MS (m/z): [M] calc'd for C₁₉H₂₄ClN₃O₃S is 409.12. found 409.93 [M+H].

### Example 93. (trans)-1-allyl-4-((5-(2-chloropyridin-4-yl)-2-methylphenyl)sulfonyl)-2,5-dimethylpiperazine (SR-35465)

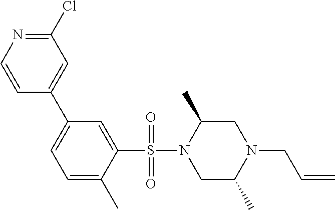 [SMILES: C=CCN1C[C@H](C)N(S(=O)(=O)c2cc(-c3ccnc(Cl)c3)ccc2C)C[C@H]1C]

This compound was prepared according to the procedure for SR-34793 in 54% overall yield starting with trans-1-allyl-2,5-dimethylpiperazine. ¹H NMR (CD₃OD) δ 8.45 (d, J=5.2 Hz, 1H), 8.32 (d, J=1.6 Hz, 1H), 7.98 (dd, J=8.0, 2.0 Hz, 1H), 7.81 (s, 1H), 7.71 (dd, J=5.2, 1.2 Hz, 1H), 7.62 (d, J=8.0 Hz, 1H), 6.01-5.91 (m, 1H), 5.67-5.61 (m, 2H), 3.81-3.76 (m, 4H), 3.66 (m, 2H), 3.45 (dd, J=13.2, 4.4 Hz, 2H), 2.65 (s, 3H), 1.30 (br, 3H), 1.22 (d, J=7.2 Hz, 3H); MS (m/z): [M] calc'd for C₂₁H₂₆ClN₃O₂S is 419.14. found 419.98 [M+H].

### Example 94. (4-((5-(2-Chloropyridin-4-yl)-2-methylphenyl)sulfonyl)piperazin-1-yl)(furan-2-yl)methanone (SR-35516)

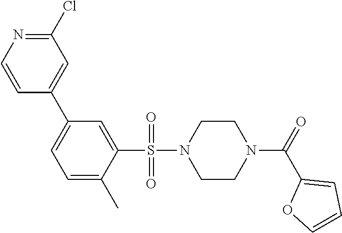 [SMILES: Cc1ccc(-c2ccnc(Cl)c2)cc1S(=O)(=O)N1CCN(C(=O)c2ccco2)CC1]

This compound was prepared according to the procedure for SR-34793 in 24% overall yield starting with 1-(2-furoyl)piperazine. ¹H NMR (CD₃OD) δ 8.45 (d, J=5.2 Hz, 1H), 8.23 (d, J=2.0 Hz, 1H), 7.98 (dd, J=8.0, 2.0 Hz, 1H), 7.80 (s, 1H), 7.70 (dd, J=5.6, 1.6 Hz, 1H), 7.66 (d, J=1.2 Hz, 1H), 7.60 (d, J=8.0 Hz, 1H), 7.04 (d, J=3.2 Hz, 1H), 6.57 (dd, J=3.6, 1.6 Hz, 1H), 3.88 (m, 8H), 2.71 (s, 3H); MS (m/z): [M] calc'd for C₂₁H₂₀ClN₃O₄S is 445.09. found 445.91 [M+H].

### Example 95: Cell Viability Assays

The tables below show the structures of specific examples of compounds useful for practice of methods of the invention, associated with corresponding data such as compound identifier, and biological results.

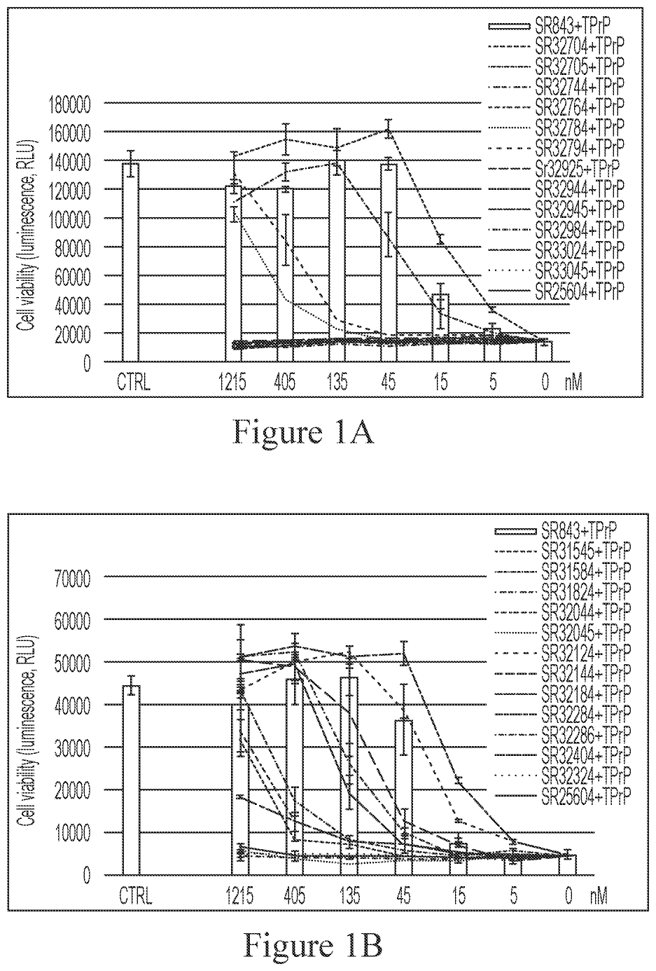

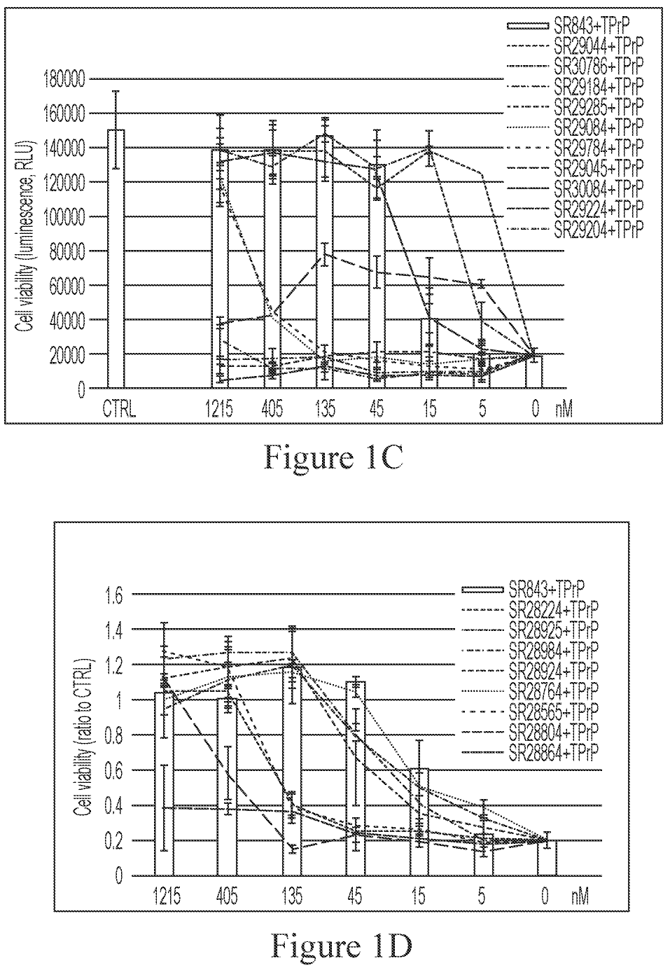

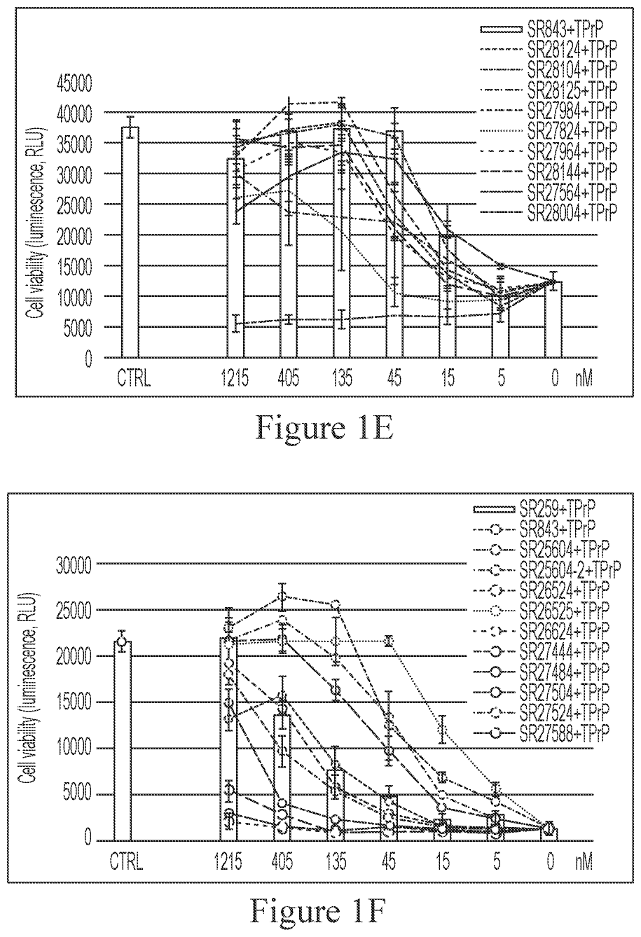

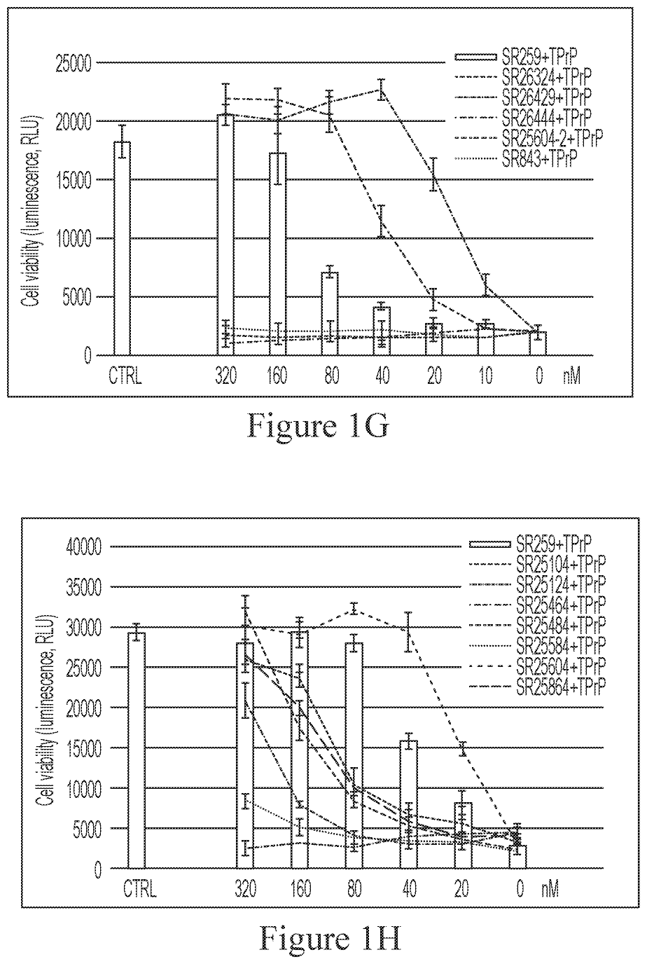

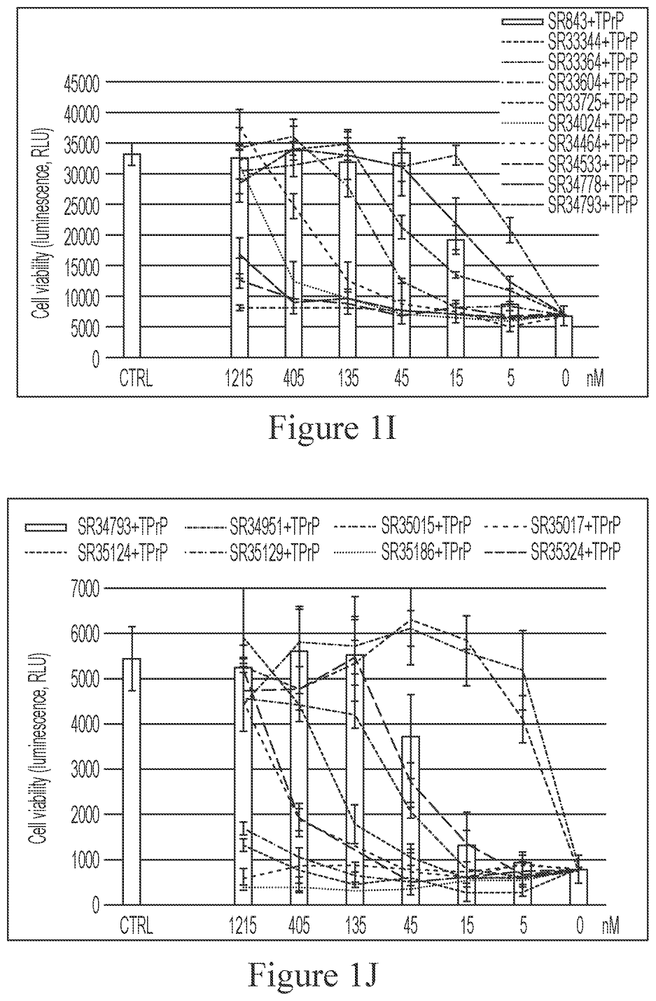

The neuroprotective activity of test compounds was quantified in a cell viability assay (CellTiter-Glo©) assessing the ability of compounds to prevent neuronal death due to NAD deprivation induced by the misfolded protein TPrP. Dose-response profiles were established in the TPrP neuroprotection assay for each compound. PK1 neuroblastoma cells (˜1000 cells/well, 96-well plates) were exposed to TPrP at 5 μg/ml and to compounds at doses ranging 2 nM up to 1.5 μM for 4 days. TPrP was prepared as described in Zhou, et. al., Proc Natl Acad Sci USA 109, 3113-3118 (2012)¹. Compounds were added at the doses indicated in 0.5% DMSO final concentration. Cell viability was measured using CellTiter-Glo© (Promega). Efficacious concentrations (EC₅₀ values) were determined. TPrP EC₅₀ for the compounds described herein are shown in Table 6. Dose-response activity curves are shown in FIGS. 1A, 1B, 1C, 1D, 1E, 1F, 1G, 1H, 1I and 1J.

### Example 96: Microsomal Stability Assays

The metabolic stability of some test compounds was determined in hepatic human and mouse microsomes. The compound was incubated with 1 mg/ml human or mouse hepatic microsomes at 37° C. with continuous shaking. Aliquots were removed at various time points between 5 minutes and 2 hours and acetonitrile was added to quench the reactions and precipitate the proteins. Samples were then centrifuged through 0.45 μm filter plates and half-lives were determined by LC-MS/MS. Microsomal stability ≥15 minutes for tested compounds is shown in Table 6.

### Example 97: NAMPT Activation Assays

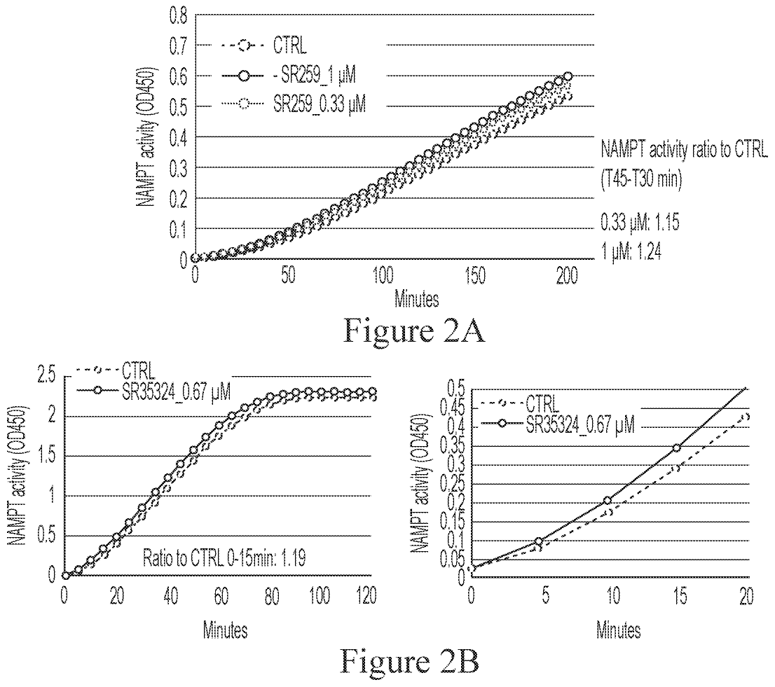

The ability of some test compounds to activate human NAMPT was tested in a colorimetric NAMPT activity assay (AbCam ab221819). The assay was performed according to the manufacturer's instructions. For compound SR259, mouse NAMPT activity was measured by replacing human NAMPT by mouse NAMPT (Fisher Scientific AG-40B0179-C050). Enzymatic activity rate was calculated by the formula: ((A at T2)−(A at T1))/(T2−T1) where A is the OD450 at each time point T (min). Examples of activation curves are shown in FIGS. 2A-2B. Activation ratios compared to baseline (CTRL, no compound) are also indicated in FIGS. 2A-2B. NAMPT activation ≥10% for tested compounds is shown in Table 6.

TABLE 6

| | | | Stability in | |
|---|---|---|---|---|
| | | | human | Human |
| | | | microsomes | NAMPT |
| | | TPrP | ≥15 | activation |
| Compound | Structure | EC50 | minutes | ≥10% |
| SR-843 | 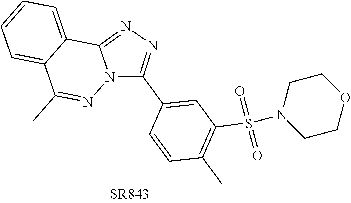 [SMILES: Cc1ccc(-c2nnc3c4ccccc4c(C)nn23)cc1S(=O)(=O)N1CCOCC1] | 15 nM, 20 nM, 25 nM, 30 nM, | | |
| SR259 | 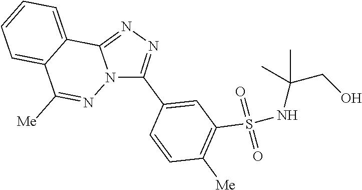 [SMILES: Cc1ccc(-c2nnc3c4ccccc4c(C)nn23)cc1S(=O)(=O)NC(C)(C)CO] | 40, 120 nM, 140 nM | | |
| SR-25604 | 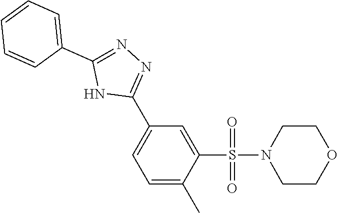 [SMILES: Cc1ccc(-c2nnc(-c3ccccc3)[nH]2)cc1S(=O)(=O)N1CCOCC1] | 35 nM, 30 nM, 40 nM | | |
| SR-32944 | 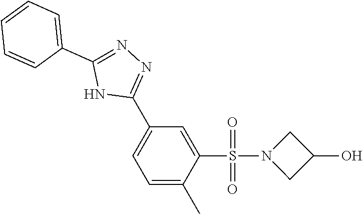 [SMILES: Cc1ccc(-c2nnc(-c3ccccc3)[nH]2)cc1S(=O)(=O)N1CC(O)C1] | 15 nM | yes | yes |
| SR-32945 | 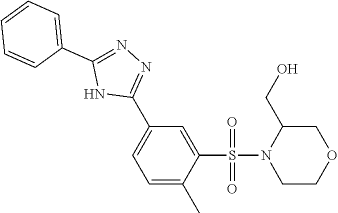 [SMILES: Cc1ccc(-c2nnc(-c3ccccc3)[nH]2)cc1S(=O)(=O)N1CCOCC1CO] | 300 nM | | |
| SR-32704 | 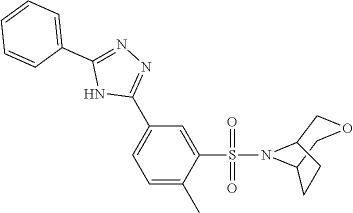 [SMILES: Cc1ccc(-c2nnc(-c3ccccc3)[nH]2)cc1S(=O)(=O)N1C2CCC1COC2] | 800 nM | | |
| SR-32705 | 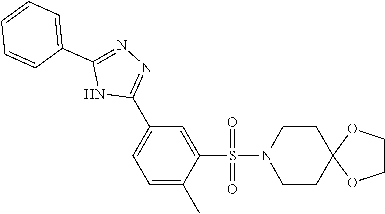 [SMILES: Cc1ccc(-c2nnc(-c3ccccc3)[nH]2)cc1S(=O)(=O)N1CCC2(CC1)OCCO2] | >1.5 μM | | |
| SR-32744 | 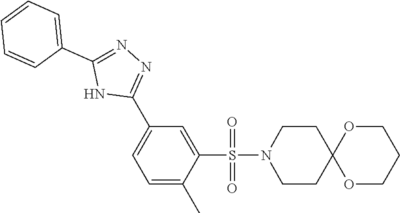 [SMILES: Cc1ccc(-c2nnc(-c3ccccc3)[nH]2)cc1S(=O)(=O)N1CCC2(CC1)OCCCO2] | >1.5 μM | | |
| SR-32764 | 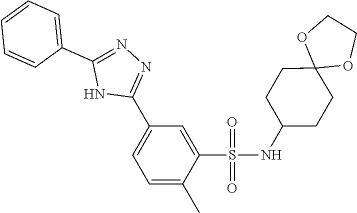 [SMILES: Cc1ccc(-c2nnc(-c3ccccc3)[nH]2)cc1S(=O)(=O)NC1CCC2(CC1)OCCO2] | >1.5 μM | | |
| SR-32784 | 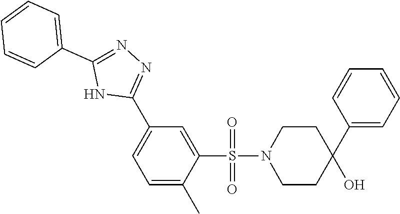 [SMILES: Cc1ccc(-c2nnc(-c3ccccc3)[nH]2)cc1S(=O)(=O)N1CCC(O)(c2ccccc2)CC1] | >1.5 μM | | |
| SR-32924 | 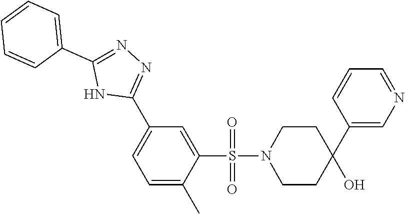 [SMILES: Cc1ccc(-c2nnc(-c3ccccc3)[nH]2)cc1S(=O)(=O)N1CCC(O)(c2cccnc2)CC1] | >1.5 μM | | |
| SR-32925 | 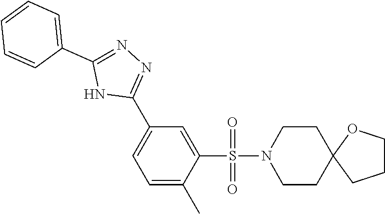 [SMILES: Cc1ccc(-c2nnc(-c3ccccc3)[nH]2)cc1S(=O)(=O)N1CCC2(CCCO2)CC1] | >1.5 μM | | |
| SR-32984 | 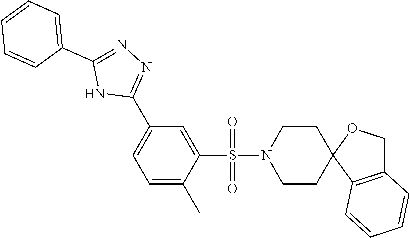 [SMILES: Cc1ccc(-c2nnc(-c3ccccc3)[nH]2)cc1S(=O)(=O)N1CCC2(CC1)OCc1ccccc12] | >1.5 μM | | |
| SR-33024 | 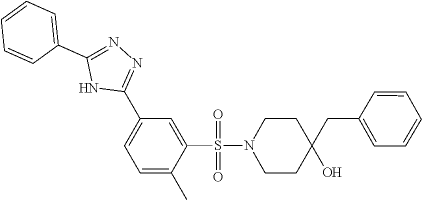 [SMILES: Cc1ccc(-c2nnc(-c3ccccc3)[nH]2)cc1S(=O)(=O)N1CCC(O)(Cc2ccccc2)CC1] | >1.5 μM | | |
| SR-33045 | 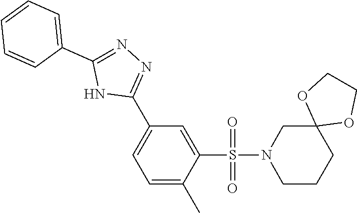 [SMILES: Cc1ccc(-c2nnc(-c3ccccc3)[nH]2)cc1S(=O)(=O)N1CCCC2(C1)OCCO2] | >1.5 μM | | |
| SR-32124 | 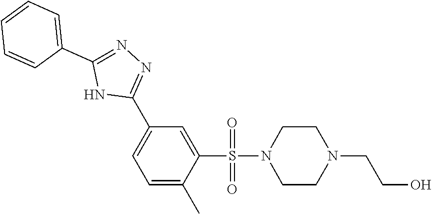 [SMILES: Cc1ccc(-c2nnc(-c3ccccc3)[nH]2)cc1S(=O)(=O)N1CCN(CCO)CC1] | 15 nM | yes | |
| SR-31584 | 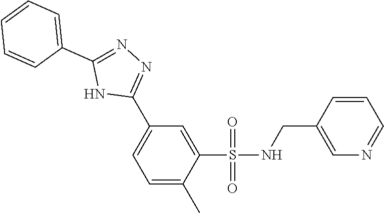 [SMILES: Cc1ccc(-c2nnc(-c3ccccc3)[nH]2)cc1S(=O)(=O)NCc1cccnc1] | 90 nM | | |
| SR-32286 | 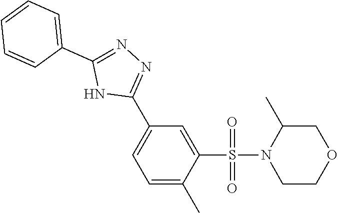 [SMILES: Cc1ccc(-c2nnc(-c3ccccc3)[nH]2)cc1S(=O)(=O)N1CCOCC1C] | 110 nM | | |
| SR-32184 | 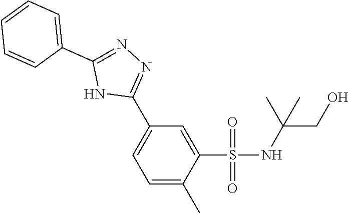 [SMILES: Cc1ccc(-c2nnc(-c3ccccc3)[nH]2)cc1S(=O)(=O)NC(C)(C)CO] | 180 nM | | |
| SR-32284 | 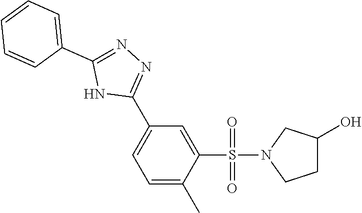 [SMILES: Cc1ccc(-c2nnc(-c3ccccc3)[nH]2)cc1S(=O)(=O)N1CCC(O)C1] | 550 nM | | |
| SR-32404 | 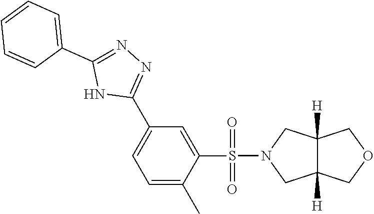 [SMILES: Cc1ccc(-c2nnc(-c3ccccc3)[nH]2)cc1S(=O)(=O)N1C[C@H]2COC[C@H]2C1] | 800 nM | | |
| SR-32324 | 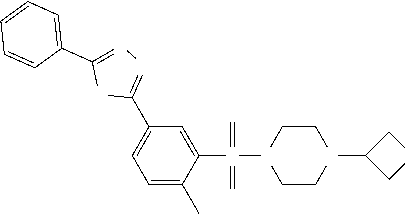 | 900 nM | | |
| SR-32044 | 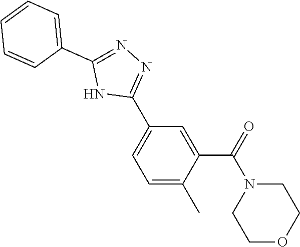 [SMILES: Cc1ccc(-c2nnc(-c3ccccc3)[nH]2)cc1C(=O)N1CCOCC1] | ~1.5 μM | | |
| SR-32045 | 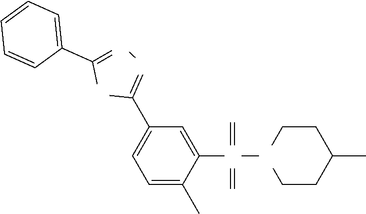 | >1.5 μM | | |
| SR-32144 | 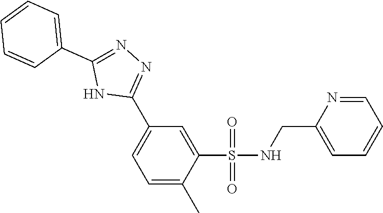 [SMILES: Cc1ccc(-c2nnc(-c3ccccc3)[nH]2)cc1S(=O)(=O)NCc1ccccn1] | >1.5 μM | | |
| SR-31824 | 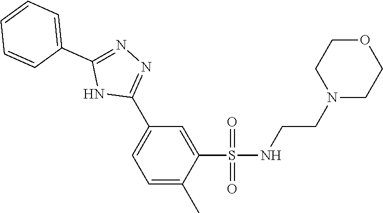 [SMILES: Cc1ccc(-c2nnc(-c3ccccc3)[nH]2)cc1S(=O)(=O)NCCN1CCOCC1] | >1.5 μM | | |
| SR-31545 | 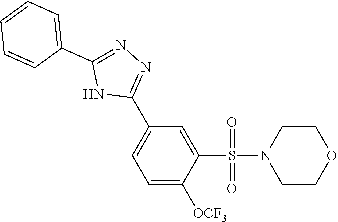 [SMILES: O=S(=O)(c1cc(-c2nnc(-c3ccccc3)[nH]2)ccc1OC(F)(F)F)N1CCOCC1] | >1.5 μM | | |
| SR-29204 | 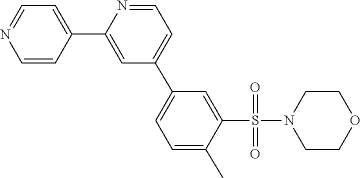 [SMILES: Cc1ccc(-c2ccnc(-c3ccncc3)c2)cc1S(=O)(=O)N1CCOCC1] | <5 nM (100% protection at 5 nM) | | |
| SR-29224 | 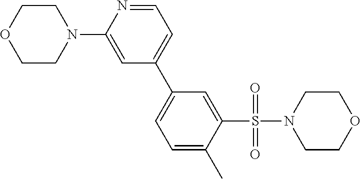 [SMILES: Cc1ccc(-c2ccnc(N3CCOCC3)c2)cc1S(=O)(=O)N1CCOCC1] | 6 nM | | |
| SR-30084 | 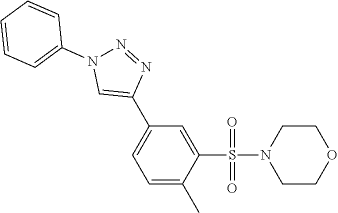 [SMILES: Cc1ccc(-c2cn(-c3ccccc3)nn2)cc1S(=O)(=O)N1CCOCC1] | 30 nM | | |
| SR-29045 | 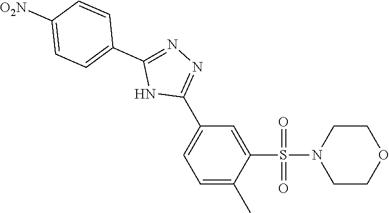 [SMILES: Cc1ccc(-c2nnc(-c3ccc([N+](=O)[O-])cc3)[nH]2)cc1S(=O)(=O)N1CCOCC1] | 5 nM (but toxic at cc >5 nM) | | |
| SR-29784 | 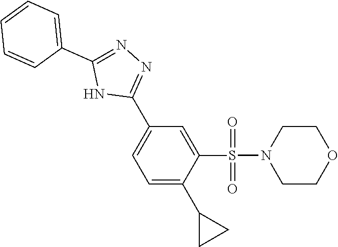 [SMILES: O=S(=O)(c1cc(-c2nnc(-c3ccccc3)[nH]2)ccc1C1CC1)N1CCOCC1] | 600 nM | | |
| SR-29084 | 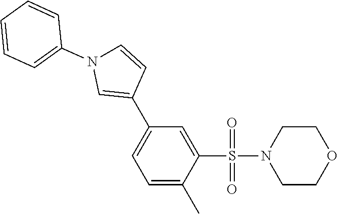 [SMILES: Cc1ccc(-c2ccn(-c3ccccc3)c2)cc1S(=O)(=O)N1CCOCC1] | 600 nM | | |
| SR-29285 | 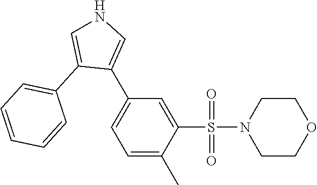 [SMILES: Cc1ccc(-c2c[nH]cc2-c2ccccc2)cc1S(=O)(=O)N1CCOCC1] | >1.2 M | | |
| SR-29044 | 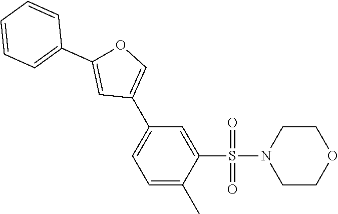 [SMILES: Cc1ccc(-c2coc(-c3ccccc3)c2)cc1S(=O)(=O)N1CCOCC1] | >1.2 M | | |
| SR-29184 | 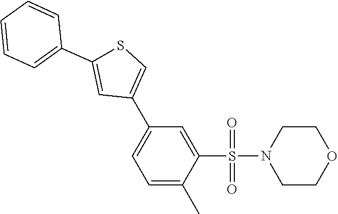 [SMILES: Cc1ccc(-c2csc(-c3ccccc3)c2)cc1S(=O)(=O)N1CCOCC1] | >1.2 M | | |
| SR-30786 | 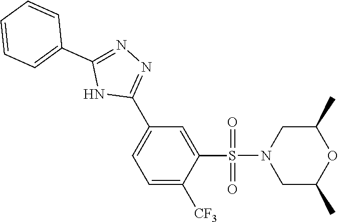 [SMILES: C[C@@H]1CN(S(=O)(=O)c2cc(-c3nnc(-c4ccccc4)[nH]3)ccc2C(F)(F)F)C[C@H](C)O1] | >1.2 M | | |
| SR-28224 | 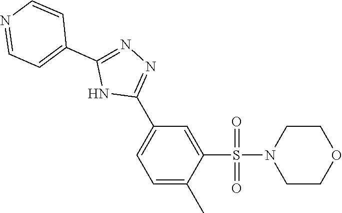 [SMILES: Cc1ccc(-c2nnc(-c3ccncc3)[nH]2)cc1S(=O)(=O)N1CCOCC1] | 18 nM | | |
| SR-28925 | 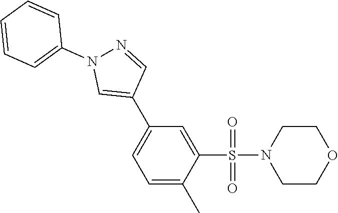 [SMILES: Cc1ccc(-c2cnn(-c3ccccc3)c2)cc1S(=O)(=O)N1CCOCC1] | 20 nM | | |
| SR-28984 | 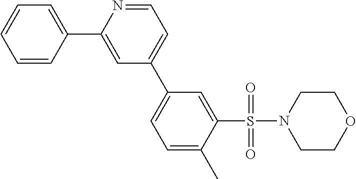 [SMILES: Cc1ccc(-c2ccnc(-c3ccccc3)c2)cc1S(=O)(=O)N1CCOCC1] | 30 nM | | |
| SR-28924 | 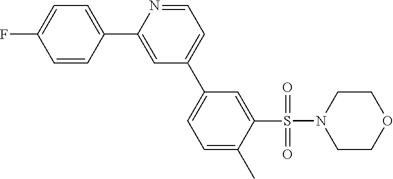 [SMILES: Cc1ccc(-c2ccnc(-c3ccc(F)cc3)c2)cc1S(=O)(=O)N1CCOCC1] | 40 nM | | |
| SR-28565 | 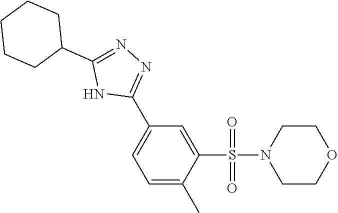 [SMILES: Cc1ccc(-c2nnc(C3CCCCC3)[nH]2)cc1S(=O)(=O)N1CCOCC1] | 150 nM | | |
| SR-28764 | 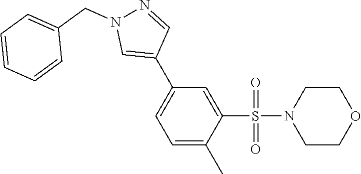 [SMILES: Cc1ccc(-c2cnn(Cc3ccccc3)c2)cc1S(=O)(=O)N1CCOCC1] | 150 nM | | |
| SR-28804 | 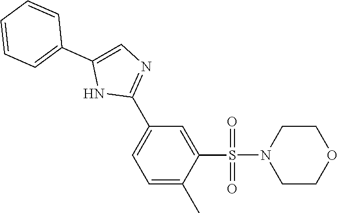 [SMILES: Cc1ccc(-c2ncc(-c3ccccc3)[nH]2)cc1S(=O)(=O)N1CCOCC1] | 400 nM | | |
| SR-28864 | 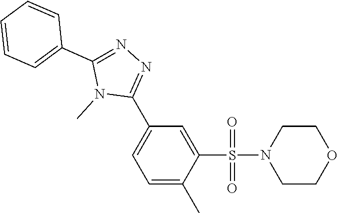 [SMILES: Cc1ccc(-c2nnc(-c3ccccc3)n2C)cc1S(=O)(=O)N1CCOCC1] | >1.2 μM | | |
| SR-28124 | 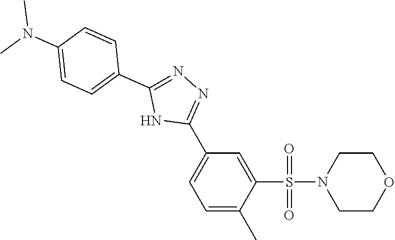 [SMILES: Cc1ccc(-c2nnc(-c3ccc(N(C)C)cc3)[nH]2)cc1S(=O)(=O)N1CCOCC1] | 12 nM (but toxic at cc >135 nM) | | |
| SR-28104 | 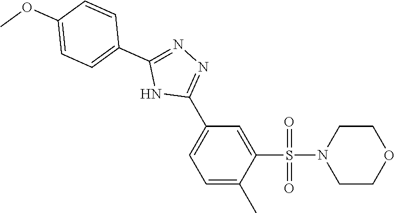 [SMILES: COc1ccc(-c2nnc(-c3ccc(C)c(S(=O)(=O)N4CCOCC4)c3)[nH]2)cc1] | 15 nM | | |
| SR-27984 | 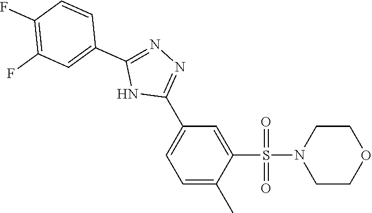 [SMILES: Cc1ccc(-c2nnc(-c3ccc(F)c(F)c3)[nH]2)cc1S(=O)(=O)N1CCOCC1] | 20 nM | | |
| SR-28125 | 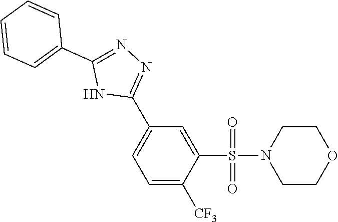 [SMILES: O=S(=O)(c1cc(-c2nnc(-c3ccccc3)[nH]2)ccc1C(F)(F)F)N1CCOCC1] | 30 nM | yes | |
| SR-27824 | 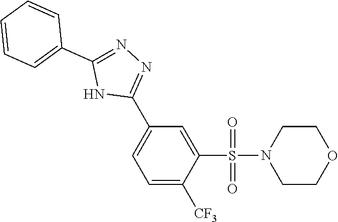 [SMILES: O=S(=O)(c1cc(-c2nnc(-c3ccccc3)[nH]2)ccc1C(F)(F)F)N1CCOCC1] | 30 nM | | |
| SR-28144 | 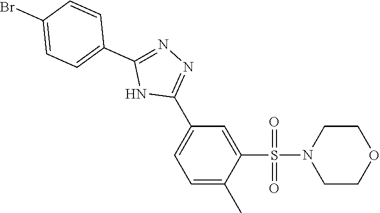 [SMILES: Cc1ccc(-c2nnc(-c3ccc(Br)cc3)[nH]2)cc1S(=O)(=O)N1CCOCC1] | 40 nM | | |
| SR-27964 | 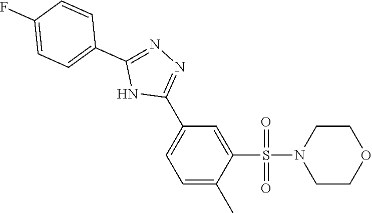 [SMILES: Cc1ccc(-c2nnc(-c3ccc(F)cc3)[nH]2)cc1S(=O)(=O)N1CCOCC1] | 40 nM | | |
| SR-27564 | 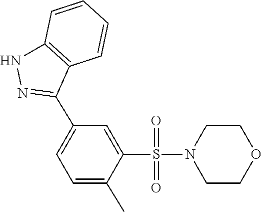 [SMILES: Cc1ccc(-c2n[nH]c3ccccc23)cc1S(=O)(=O)N1CCOCC1] | 135 nM | | |
| SR-28004 | 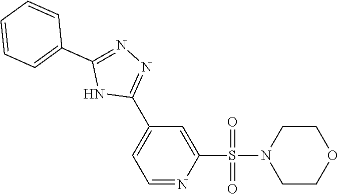 [SMILES: O=S(=O)(c1cc(-c2nnc(-c3ccccc3)[nH]2)ccn1)N1CCOCC1] | >1.2 μM | | |
| SR-25604- 2 | 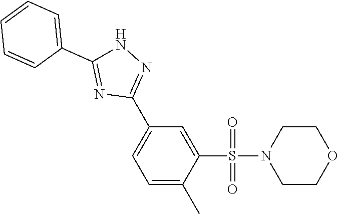 [SMILES: Cc1ccc(-c2n[nH]c(-c3ccccc3)n2)cc1S(=O)(=O)N1CCOCC1] | 40 nM | | |
| SR-26524 | 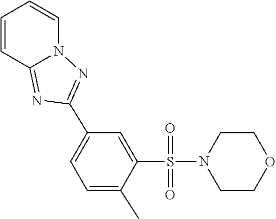 [SMILES: Cc1ccc(-c2nc3ccccn3n2)cc1S(=O)(=O)N1CCOCC1] | 50 nM | | |
| SR-26525 | 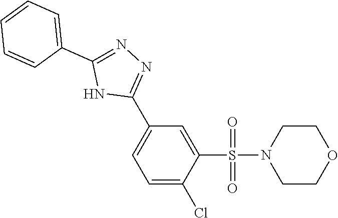 [SMILES: O=S(=O)(c1cc(-c2nnc(-c3ccccc3)[nH]2)ccc1Cl)N1CCOCC1] | 100 nM | | |
| SR-27558 | 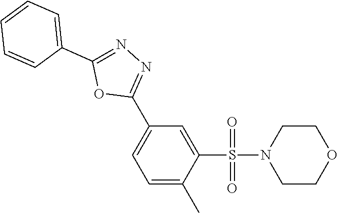 [SMILES: Cc1ccc(-c2nnc(-c3ccccc3)o2)cc1S(=O)(=O)N1CCOCC1] | 140 nM | | |
| SR-27524 | 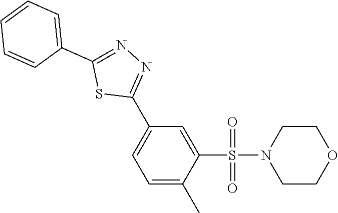 [SMILES: Cc1ccc(-c2nnc(-c3ccccc3)s2)cc1S(=O)(=O)N1CCOCC1] | 180 nM | | |
| SR-26624 | 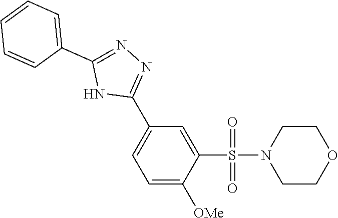 [SMILES: COc1ccc(-c2nnc(-c3ccccc3)[nH]2)cc1S(=O)(=O)N1CCOCC1] | 260 nM | | |
| SR-27444 | 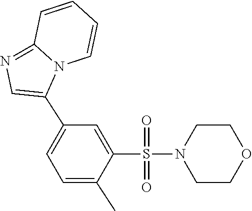 [SMILES: Cc1ccc(-c2cnc3ccccn23)cc1S(=O)(=O)N1CCOCC1] | >320 nM | | |
| SR-27484 | 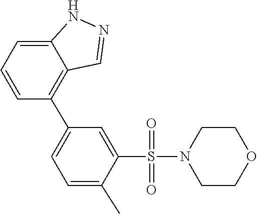 [SMILES: Cc1ccc(-c2cccc3[nH]ncc23)cc1S(=O)(=O)N1CCOCC1] | >320 nM | | |
| SR-27504 | 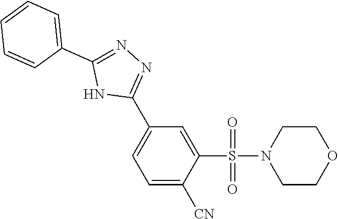 [SMILES: N#Cc1ccc(-c2nnc(-c3ccccc3)[nH]2)cc1S(=O)(=O)N1CCOCC1] | >320 nM | | |
| SR-26324 | 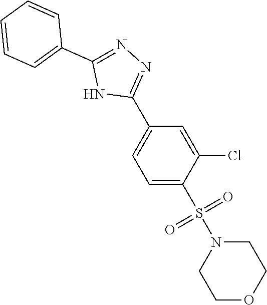 [SMILES: O=S(=O)(c1ccc(-c2nnc(-c3ccccc3)[nH]2)cc1Cl)N1CCOCC1] | >320 nM | | |
| SR-26424 | 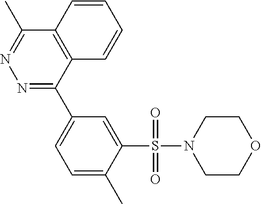 [SMILES: Cc1ccc(-c2nnc(C)c3ccccc23)cc1S(=O)(=O)N1CCOCC1] | >320 nM | | |
| SR-26444 | 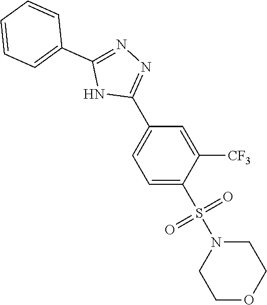 [SMILES: O=S(=O)(c1ccc(-c2nnc(-c3ccccc3)[nH]2)cc1C(F)(F)F)N1CCOCC1] | >320 nM | | |
| SR-25104 | 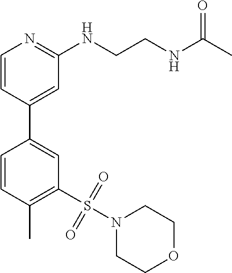 [SMILES: CC(=O)NCCNc1cc(-c2ccc(C)c(S(=O)(=O)N3CCOCC3)c2)ccn1] | 100 nM | | |
| SR-25864 | 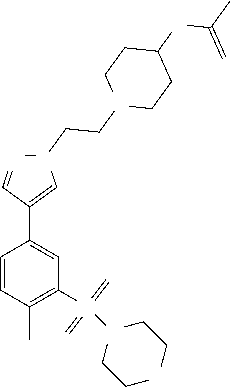 | 120 nM | | yes |
| SR-25484 | 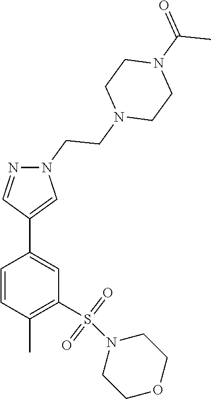 [SMILES: CC(=O)N1CCN(CCn2cc(-c3ccc(C)c(S(=O)(=O)N4CCOCC4)c3)cn2)CC1] | 140 nM | yes | |
| SR-25124 | 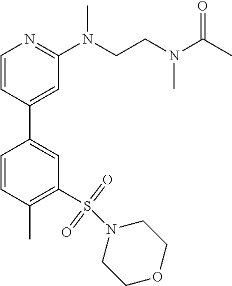 [SMILES: CC(=O)N(C)CCN(C)c1cc(-c2ccc(C)c(S(=O)(=O)N3CCOCC3)c2)ccn1] | 240 nM | | |
| SR-25584 | 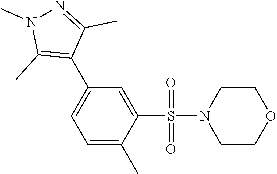 [SMILES: Cc1ccc(-c2c(C)nn(C)c2C)cc1S(=O)(=O)N1CCOCC1] | >320 nM | | |
| SR-25464 | 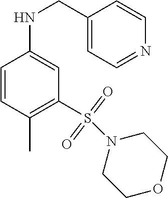 [SMILES: Cc1ccc(NCc2ccncc2)cc1S(=O)(=O)N1CCOCC1] | >320 nM | | |
| SR-24583 | 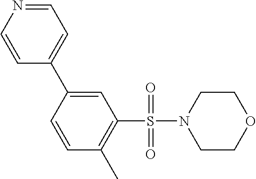 [SMILES: Cc1ccc(-c2ccncc2)cc1S(=O)(=O)N1CCOCC1] | 100 nM | | |
| SR-24578 | 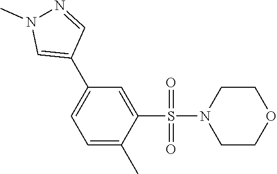 [SMILES: Cc1ccc(-c2cnn(C)c2)cc1S(=O)(=O)N1CCOCC1] | 100 nM | | |
| SR-24581 | 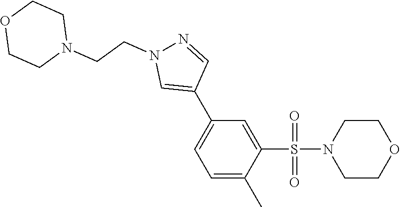 [SMILES: Cc1ccc(-c2cnn(CCN3CCOCC3)c2)cc1S(=O)(=O)N1CCOCC1] | 100 nM | | |
| SR-35422 | 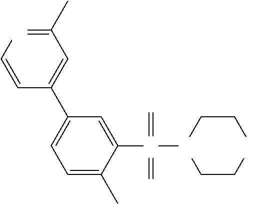 | <5 nM | | |
| SR-35186 | 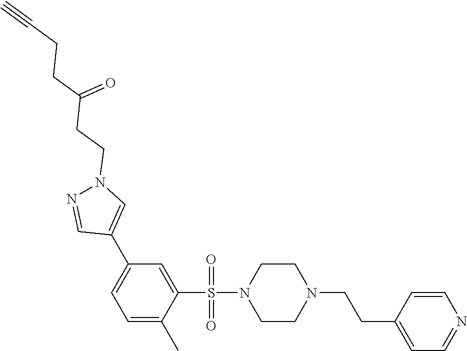 [SMILES: C#CCCC(=O)CCn1cc(-c2ccc(C)c(S(=O)(=O)N3CCN(CCc4ccncc4)CC3)c2)cn1] | <5 nM | | |
| SR-34793 | 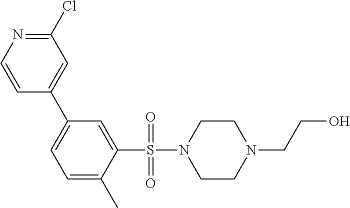 [SMILES: Cc1ccc(-c2ccnc(Cl)c2)cc1S(=O)(=O)N1CCN(CCO)CC1] | 40 nM | yes | |
| SR-35015 | 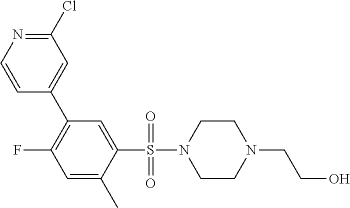 [SMILES: Cc1cc(F)c(-c2ccnc(Cl)c2)cc1S(=O)(=O)N1CCN(CCO)CC1] | 50 nM | yes | |
| SR-34951 | 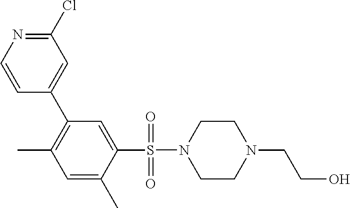 [SMILES: Cc1cc(C)c(S(=O)(=O)N2CCN(CCO)CC2)cc1-c1ccnc(Cl)c1] | 90 nM | | |
| SR-35516 | 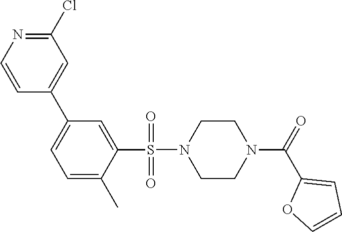 [SMILES: Cc1ccc(-c2ccnc(Cl)c2)cc1S(=O)(=O)N1CCN(C(=O)c2ccco2)CC1] | 270 nM | | |
| SR-35464 | 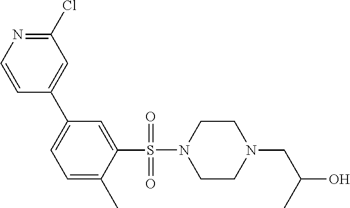 [SMILES: Cc1ccc(-c2ccnc(Cl)c2)cc1S(=O)(=O)N1CCN(CC(C)O)CC1] | 800 nM | | |
| SR-35017 | 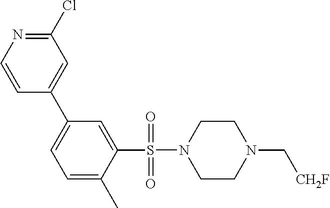 | 900 nM | | |
| SR-35129 | 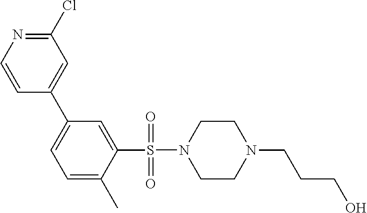 [SMILES: Cc1ccc(-c2ccnc(Cl)c2)cc1S(=O)(=O)N1CCN(CCCO)CC1] | >1.5 μM | | |
| SR-35124 | 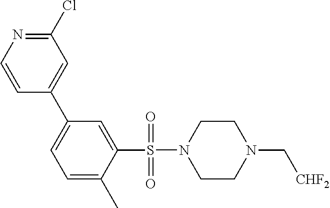 [SMILES: Cc1ccc(-c2ccnc(Cl)c2)cc1S(=O)(=O)N1CCN(CC(F)F)CC1] | >1.5 μM | | |
| SR-35465 | 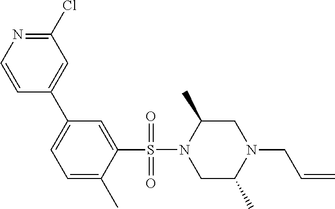 [SMILES: C=CCN1C[C@H](C)N(S(=O)(=O)c2cc(-c3ccnc(Cl)c3)ccc2C)C[C@H]1C] | >1.5 μM | | |
| SR-35324 | 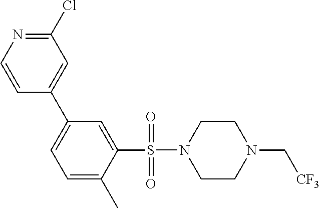 [SMILES: Cc1ccc(-c2ccnc(Cl)c2)cc1S(=O)(=O)N1CCN(CC(F)(F)F)CC1] | >1.5 μM | | yes |
| SR-33364 | 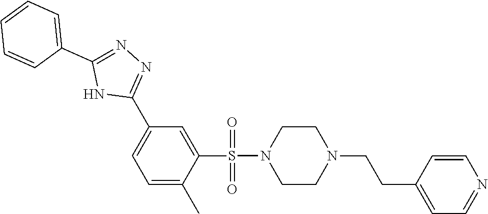 [SMILES: Cc1ccc(-c2nnc(-c3ccccc3)[nH]2)cc1S(=O)(=O)N1CCN(CCc2ccncc2)CC1] | 5 nM | | |
| SR-34778 | 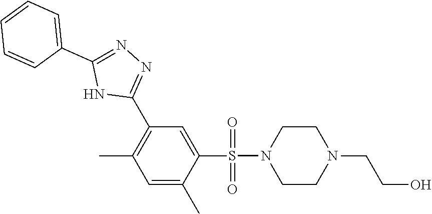 [SMILES: Cc1cc(C)c(S(=O)(=O)N2CCN(CCO)CC2)cc1-c1nnc(-c2ccccc2)[nH]1] | 13 nM | yes | yes |
| SR-34793 | 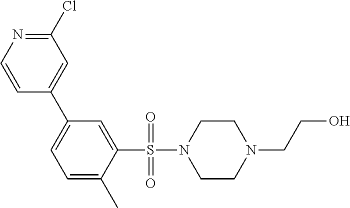 [SMILES: Cc1ccc(-c2ccnc(Cl)c2)cc1S(=O)(=O)N1CCN(CCO)CC1] | 40 nM | yes | |
| SR-33604 | 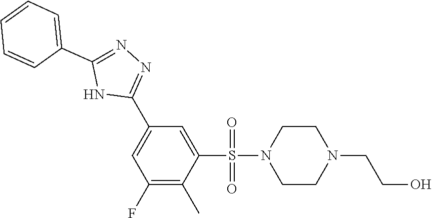 [SMILES: Cc1c(F)cc(-c2nnc(-c3ccccc3)[nH]2)cc1S(=O)(=O)N1CCN(CCO)CC1] | 90 nM | yes | yes |
| SR-34464 | 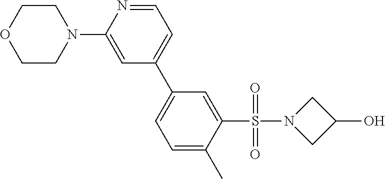 [SMILES: Cc1ccc(-c2ccnc(N3CCOCC3)c2)cc1S(=O)(=O)N1CC(O)C1] | 300 nM | yes | |
| SR-33344 | 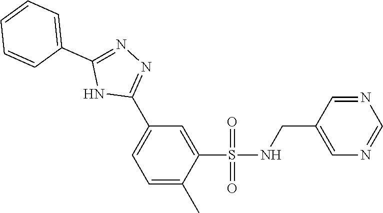 [SMILES: Cc1ccc(-c2nnc(-c3ccccc3)[nH]2)cc1S(=O)(=O)NCc1cncnc1] | 650 nM | | |
| SR-34024 | 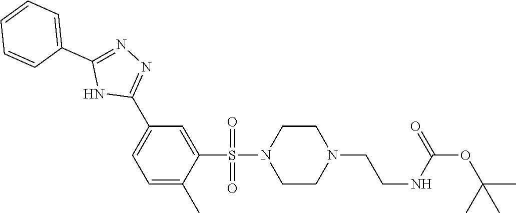 [SMILES: Cc1ccc(-c2nnc(-c3ccccc3)[nH]2)cc1S(=O)(=O)N1CCN(CCNC(=O)OC(C)(C)C)CC1] | ~1.5 μM | | |
| SR-34533 | 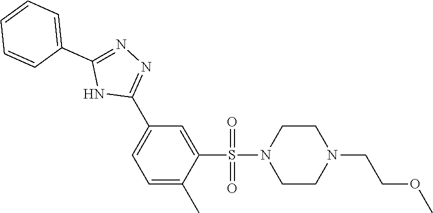 [SMILES: COCCN1CCN(S(=O)(=O)c2cc(-c3nnc(-c4ccccc4)[nH]3)ccc2C)CC1] | >1.5 μM | | |
| SR-33725 | 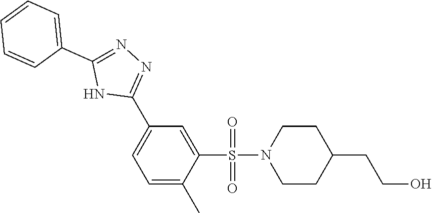 [SMILES: Cc1ccc(-c2nnc(-c3ccccc3)[nH]2)cc1S(=O)(=O)N1CCC(CCO)CC1] | >1.5 μM | | yes |

### REFERENCES

1. Zhou M, Ottenberg G, Sferrazza G F, Lasmezas C I. Highly neurotoxic monomeric alpha-helical prion protein. Proc Natl Acad Sci USA. 2012; 109(8):3113-8. Epub 2012 Feb. 11. doi: 10.1073/pnas.1118090109. PubMed PMID: 22323583; PMCID: 3286986.
2. Zhou M, Ottenberg G, Sferrazza G F, Hubbs C, Fallahi M, Rumbaugh G, Brantley A F, Lasmdzas C I. Neuronal death induced by misfolded prion protein is due to NAD+ depletion and can be relieved in vitro and in vivo by NAD+ replenishment. Brain. 2015; 138(4):992-1008. doi: 10.1093/brain/awv002; PMCID: 25678560.
3. Schondorf D C, Ivanyuk D, Baden P, Sanchez-Martinez A, De Cicco S, Yu C, Giunta I, Schwarz L K, Di Napoli G, Panagiotakopoulou V, Nestel S, Keatinge M, Pruszak J, Bandmann O, Heimrich B, Gasser T, Whitworth A J, Deleidi M. The NAD+ Precursor Nicotinamide Riboside Rescues Mitochondrial Defects and Neuronal Loss in iPSC and Fly Models of Parkinson's Disease. Cell Rep. 2018; 23(10):2976-88. Epub 2018 Jun. 7. doi: 10.1016/j.celrep.2018.05.009. PubMed PMID: 29874584.
4. Shan C, Gong Y L, Zhuang Q Q, Hou Y F, Wang S M, Zhu Q, Huang G R, Tao B, Sun L H, Zhao H Y, Li S T, Liu J M. Protective effects of beta-nicotinamide adenine dinucleotide against motor deficits and dopaminergic neuronal damage in a mouse model of Parkinson's disease. Prog Neuropsychopharmacol Biol Psychiatry. 2019; 94:109670. Epub 2019 Jun. 21. doi: 10.1016/j.pnpbp.2019.109670. PubMed PMID: 31220519.
5. Sorrentino V, Romani M, Mouchiroud L, Beck J S, Zhang H, D'Amico D, Moullan N, Potenza F, Schmid A W, Rietsch S, Counts S E, Auwerx J. Enhancing mitochondrial proteostasis reduces amyloid-beta proteotoxicity. Nature. 2017; 552(7684):187-93. Epub 2017 Dec. 7. doi: 10.1038/nature25143. PubMed PMID: 29211722; PMCID: PMC5730497.
6. Hou Y, Lautrup S, Cordonnier S, Wang Y, Croteau D L, Zavala E, Zhang Y, Moritoh K, O'Connell J F, Baptiste B A, Stevnsner T V, Mattson M P, Bohr V A. NAD(+) supplementation normalizes key Alzheimer's features and DNA damage responses in a new A D mouse model with introduced DNA repair deficiency. Proc Natl Acad Sci USA. 2018; 115(8):E1876-E85. doi: 10.1073/pnas.1718819115. PubMed PMID: 29432159; PMCID: PMC5828618.
7. Hosseini L, Mahmoudi J, Pashazadeh F, Salehi-Pourmehr H, Sadigh-Eteghad S. Protective Effects of Nicotinamide Adenine Dinucleotide and Related Precursors in Alzheimer's Disease: A Systematic Review of Preclinical Studies. J Mol Neurosci. 2021; 71(7):1425-35. Epub 20210428. doi: 10.1007/s12031-021-01842-6. PubMed PMID: 33907963.
8. Wang X, He H J, Xiong X, Zhou S, Wang W W, Feng L, Han R, Xie C L. NAD(+) in Alzheimer's Disease: Molecular Mechanisms and Systematic Therapeutic Evidence Obtained in vivo. Front Cell Dev Biol. 2021; 9:668491. Epub 20210803. doi: 10.3389/fcell.2021.668491. PubMed PMID: 34414179; PMCID: PMC8369418.
9. Harlan B A, Killoy K M, Pehar M, Liu L, Auwerx J, Vargas M R. Evaluation of the NAD(+) biosynthetic pathway in ALS patients and effect of modulating NAD(+) levels in hSOD1-linked ALS mouse models. Exp Neurol. 2020; 327:113219. Epub 2020 Feb. 6. doi: 10.1016/j.expneurol.2020.113219. PubMed PMID: 32014438; PMCID: PMC7089832.
10. Harlan B A, Pehar M, Sharma D R, Beeson G, Beeson C C, Vargas M R. Enhancing NAD+ Salvage Pathway Reverts the Toxicity of Primary Astrocytes Expressing Amyotrophic Lateral Sclerosis-linked Mutant Superoxide Dismutase 1 (SOD1). J Biol Chem. 2016; 291(20):10836-46. doi: 10.1074/jbc.M115.698779. PubMed PMID: 27002158; PMCID: PMC4865928.
11. Massudi H, Grant R, Braidy N, Guest J, Farnsworth B, Guillemin G J. Age-associated changes in oxidative stress and NAD+ metabolism in human tissue. PLoS One. 2012; 7(7):e42357. Epub 2012 Aug. 1. doi: 10.1371/journal.pone.0042357. PubMed PMID: 22848760; PMCID: 3407129.
12. Zhang H, Ryu D, Wu Y, Gariani K, Wang X, Luan P, D'Amico D, Ropelle E R, Lutolf M P, Aebersold R, Schoonjans K, Menzies K J, Auwerx J. NAD(+) repletion improves mitochondrial and stem cell function and enhances life span in mice. Science. 2016; 352(6292):1436-43. Epub 2016/04/30. doi: 10.1126/science.aaf2693. PubMed PMID: 27127236.
13. Mouchiroud L, Houtkooper R H, Moullan N, Katsyuba E, Ryu D, Canto C, Mottis A, Jo Y S, Viswanathan M, Schoonjans K, Guarente L, Auwerx J. The NAD(+)/Sirtuin Pathway Modulates Longevity through Activation of Mitochondrial UPR and FOXO Signaling. Cell. 2013; 154(2):430-41. doi: 10.1016/j.cell.2013.06.016. PubMed PMID: 23870130; PMCID: PMC3753670.
14. Penberthy W T, Tsunoda I. The importance of NAD in multiple sclerosis. Curr Pharm Des. 2009; 15(1):64-99. Epub 2009 Jan. 20. PubMed PMID: 19149604; PMCID: 2651433.
15. Satchell M A, Zhang X, Kochanek P M, Dixon C E, Jenkins L W, Melick J, Szabo C, Clark R S. A dual role for poly-ADP-ribosylation in spatial memory acquisition after traumatic brain injury in mice involving NAD+ depletion and ribosylation of 14-3-3gamma. J Neurochem. 2003; 85(3):697-708. PubMed PMID: 12694396.
16. Brown K D, Maqsood S, Huang J Y, Pan Y, Harkcom W, Li W, Sauve A, Verdin E, Jaffrey S R. Activation of SIRT3 by the NAD(+) precursor nicotinamide riboside protects from noise-induced hearing loss. Cell Metab. 2014; 20(6):1059-68. Epub 2014 Dec. 4. doi: 10.1016/j.cmet.2014.11.003. PubMed PMID: 25470550; PMCID: PMC4940130.
17. Vaur P, Brugg B, Mericskay M, Li Z, Schmidt M S, Vivien D, Orset C, Jacotot E, Brenner C, Duplus E. Nicotinamide riboside, a form of vitamin B3, protects against excitotoxicity-induced axonal degeneration. FASEB J. 2017; 31(12):5440-52. doi: 10.1096/fj.201700221RR. PubMed PMID: 28842432.
18. Sasaki Y, Araki T, Milbrandt J. Stimulation of nicotinamide adenine dinucleotide biosynthetic pathways delays axonal degeneration after axotomy. J Neurosci. 2006; 26(33):8484-91. Epub 2006 Aug. 18. doi: 10.1523/JNEUROSCI.2320-06.2006. PubMed PMID: 16914673.
19. Ying W, Wei G, Wang D, Wang Q, Tang X, Shi J, Zhang P, Lu H. Intranasal administration with NAD+ profoundly decreases brain injury in a rat model of transient focal ischemia. Front Biosci. 2007; 12:2728-34. Epub 2006 Nov. 28. doi: 2267 [pii]. PubMed PMID: 17127275.
20. Hsu C P, Oka S, Shao D, Hariharan N, Sadoshima J. Nicotinamide phosphoribosyltransferase regulates cell survival through NAD+ synthesis in cardiac myocytes. Circ Res. 2009; 105(5):481-91. doi: 10.1161/CIRCRESAHA.109.203703. PubMed PMID: 19661458; PMCID: PMC2765790.
21. Yamamoto T, Byun J, Zhai P, Ikeda Y, Oka S, Sadoshima J. Nicotinamide mononucleotide, an intermediate of NAD+ synthesis, protects the heart from ischemia and reperfusion. PLoS One. 2014; 9(6):e98972. doi: 10.1371/journal.pone.0098972. PubMed PMID: 24905194; PMCID: PMC4048236.
22. Zhang M, Jiang N, Chu Y, Postnikova O, Varghese R, Horvath A, Cheema A K, Golestaneh N. Dysregulated metabolic pathways in age-related macular degeneration. Sci Rep. 2020; 10(1):2464. Epub 2020 Feb. 14. doi: 10.1038/s41598-020-59244-4. PubMed PMID: 32051464; PMCID: PMC7016007.
23. Lin J B, Kubota S, Ban N, Yoshida M, Santeford A, Sene A, Nakamura R, Zapata N, Kubota M, Tsubota K, Yoshino J, Imai S I, Apte R S. NAMPT-Mediated NAD(+) Biosynthesis Is Essential for Vision In Mice. Cell Rep. 2016; 17(1):69-85. doi: 10.1016/j.celrep.2016.08.073. PubMed PMID: 27681422; PMCID: PMC5104206.
24. Bai S, Sheline C T. NAD(+) maintenance attenuates light induced photoreceptor degeneration. Exp Eye Res. 2013; 108:76-83. Epub 2013 Jan. 1. doi: 10.1016/j.exer.2012.12.007. PubMed PMID: 23274583; PMCID: PMC3578030.
25. Zhang X, Henneman N F, Girardot P E, Sellers J T, Chrenek M A, Li Y, Wang J, Brenner C, Nickerson J M, Boatright J H. Systemic Treatment With Nicotinamide Riboside Is Protective in a Mouse Model of Light-Induced Retinal Degeneration. Invest Ophthalmol Vis Sci. 2020; 61(10):47. Epub 2020 Aug. 28. doi: 10.1167/iovs.61.10.47. PubMed PMID: 32852543; PMCID: PMC7452859.
26. Yoshino J, Mills K F, Yoon M J, Imai S. Nicotinamide mononucleotide, a key NAD(+) intermediate, treats the pathophysiology of diet- and age-induced diabetes in mice. Cell Metab. 2011; 14(4):528-36. doi: 10.1016/j.cmet.2011.08.014. PubMed PMID: 21982712; PMCID: PMC3204926.
27. Trammell S A, Weidemann B J, Chadda A, Yorek M S, Holmes A, Coppey L J, Obrosov A, Kardon R H, Yorek M A, Brenner C. Nicotinamide Riboside Opposes Type 2 Diabetes and Neuropathy in Mice. Sci Rep. 2016; 6:26933. Epub 2016 May 28. doi: 10.1038/srep26933. PubMed PMID: 27230286; PMCID: PMC4882590.
28. Costes S. Targeting protein misfolding to protect pancreatic beta-cells in type 2 diabetes. Curr Opin Pharmacol. 2018; 43:104-10. Epub 2018/09/25. doi: 10.1016/j.coph.2018.08.016. PubMed PMID: 30245473.
29. Ueberberg S, Nauck M A, Uhl W, Montemurro C, Tannapfel A, Clark A, Meier J J. Islet amyloid in patients with diabetes due to exocrine pancreatic disorders, type 2 diabetes and non-diabetic patients. J Clin Endocrinol Metab. 2020. Epub 2020/04/10. doi: 10.1210/clinem/dgaal76. PubMed PMID: 32271378.
30. Liu M, Hodish I, Haataja L, Lara-Lemus R, Rajpal G, Wright J, Arvan P. Proinsulin misfolding and diabetes: mutant INS gene-induced diabetes of youth. Trends Endocrinol Metab. 2010; 21(11):652-9. Epub 2010 Aug. 21. doi: 10.1016/j.tem.2010.07.001. PubMed PMID: 20724178; PMCID: PMC2967602.
31. Sun J, Cui J, He Q, Chen Z, Arvan P, Liu M. Proinsulin misfolding and endoplasmic reticulum stress during the development and progression of diabetes. Mol Aspects Med. 2015; 42:105-18. Epub 2015 Jan. 13. doi: 10.1016/j.mam.2015.01.001. PubMed PMID: 25579745; PMCID: PMC4404191.
32. Ralto K M, Rhee E P, Parikh S M. NAD(+) homeostasis in renal health and disease. Nat Rev Nephrol. 2020; 16(2):99-111. Epub 2019 Nov. 2. doi: 10.1038/s41581-019-0216-6. PubMed PMID: 31673160.
33. Dahlin J L, Nissink J W, Strasser J M, Francis S, Higgins L, Zhou H, Zhang Z, Walters M A. PAINS in the assay: chemical mechanisms of assay interference and promiscuous enzymatic inhibition observed during a sulfhydryl-scavenging HTS. J Med Chem. 2015; 58(5):2091-113. doi: 10.1021/jm5019093. PubMed PMID: 25634295; PMCID: PMC4360378.
34. Lipinski C A, Lombardo F, Dominy B W, Feeney P J. Experimental and computational approaches to estimate solubility and permeability in drug discovery and development settings. Adv Drug Deliv Rev. 2001; 46(1-3):3-26. Epub 2001/03/22. PubMed PMID: 11259830.
35. Veber D F, Johnson S R, Cheng H Y, Smith B R, Ward K W, Kopple K D. Molecular properties that influence the oral bioavailability of drug candidates. J Med Chem. 2002; 45(12):2615-23. Epub 2002 May 31. PubMed PMID: 12036371.

1. A compound having a structure of Formula (X), 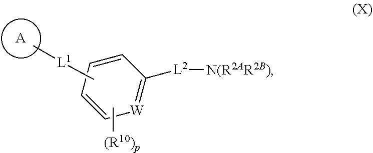
or a pharmaceutically acceptable salt thereof,
wherein:
Ring A is a substituted or unsubstituted heteroaryl,
W is —CR′═ or —N═;
L¹ is a bond, substituted or unsubstituted alkylene, or substituted or unsubstituted heteroalkylene;
L² is —S(O)₂—, or —C(O)—;
R¹ is hydrogen, —CX¹₃, —CHX₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl;
R¹⁰ is independently halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl;
p is an integer of 0 to 3;
X¹ is —F, —Br, —Cl, or —I;
R^{1A} is hydrogen, or substituted or unsubstituted alkyl;
Each R^{2A} and R^{2B} is independently selected from hydrogen, substituted or unsubstituted alkyl, substituted or unsubstituted heteroalkyl, substituted or unsubstituted cycloalkyl, or substituted or unsubstituted heterocycloalkyl; or R^{2A} and R^{2B} together with the nitrogen atom form a substituted or unsubstituted heterocycloalkyl, or substituted or unsubstituted heteroaryl.

2. The compound of claim 1, wherein:
each R^{2A} and R^{2B} is independently selected from hydrogen, substituted or unsubstituted C₁-C₄ alkyl, substituted or unsubstituted C₆-C₁₂ cycloalkyl, or substituted or unsubstituted 4 to 12 membered heterocycloalkyl; or
R^{2A} and R^{2B} together with the nitrogen atom form a substituted or unsubstituted 4 to 12 membered heterocycloalkyl, or a substituted or unsubstituted 5 to 12 membered heteroaryl.

3. The compound of any one of claims 1 to 2, wherein L¹ is a bond, unsubstituted C₁-C₄ alkylene, or unsubstituted 2 to 4 membered heteroalkylene.

4. The compound of any one of claims 1 to 3, wherein the compound has a structure of Formula (XI) or (XI′), 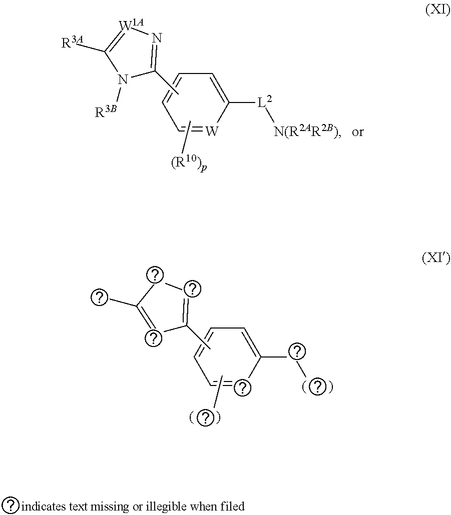
wherein:
W^{1A} is —N═, or —CR^{3C}═;
W^{1B} is —NH—, or —CR^{3A}R^{3C}—; and
Each R^{3A}, R^{3B}, and R^{3C} is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
provided that when W^{1A} is —CR^{3C}═ and R^{3C} is hydrogen, then R^{2A} and R^{2B} together with the nitrogen atom form a substituted or unsubstituted heterocycloalkyl, or substituted or unsubstituted heteroaryl.

5. The compound of claim 4, wherein R^{3A} is a substituted or unsubstituted C₅-C₆ cycloalkyl, substituted or unsubstituted phenyl, or substituted or unsubstituted pyridyl.

6. The compound of any one of claims 4 to 5, wherein the compound has the structure of Formula (XI-a), (XI′-a), (XI-b), (XI-c), (XI-d), (XI-e), (XI-f), or (XI-g), 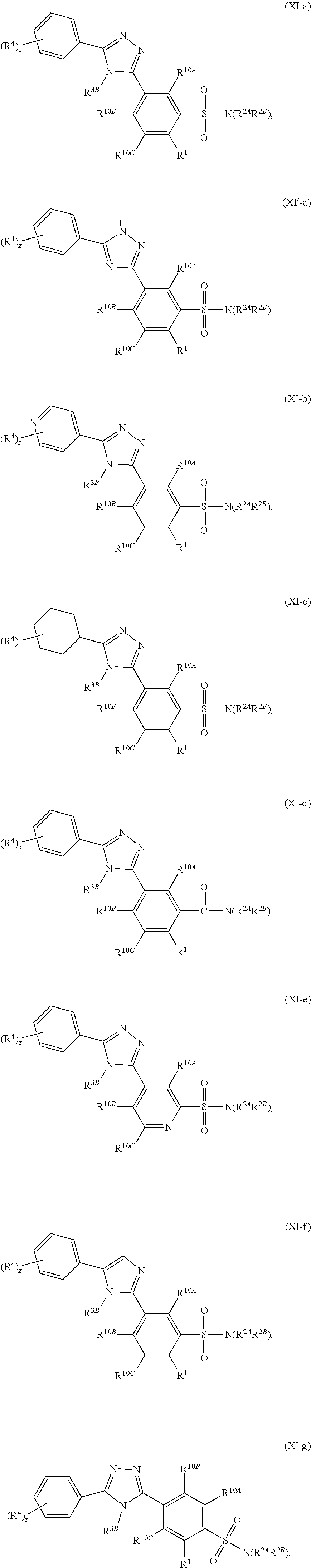
wherein:
Each R^{10A}, R^{10B}, and R^{10C} is independently hydrogen, halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl;
z is an integer of 0 to 5;
each R⁴ is independently halogen, —OR^{4A}, —NR^{4B}R^{4C}, —NO₂, substituted or unsubstituted alkyl, substituted or unsubstituted heteroalkyl; and
each R^{4A}, R^{4B} and R^{4C} is independently hydrogen, or substituted or unsubstituted alkyl.

7. The compound of any one of claims 4 to 6, wherein R^{3B} is hydrogen or —CH₃.

8. The compound of any one of claims 4 to 7, wherein z is an integer of 0 to 2; and R⁴ is —F, —Br, —Cl, —OH, —OCH₃, —NH₂, —N(CH₃)₂, or —NO₂.

9. The compound of any one of claims 4 to 8, wherein R^{2A} and R^{2B} together with the nitrogen attached thereto form a 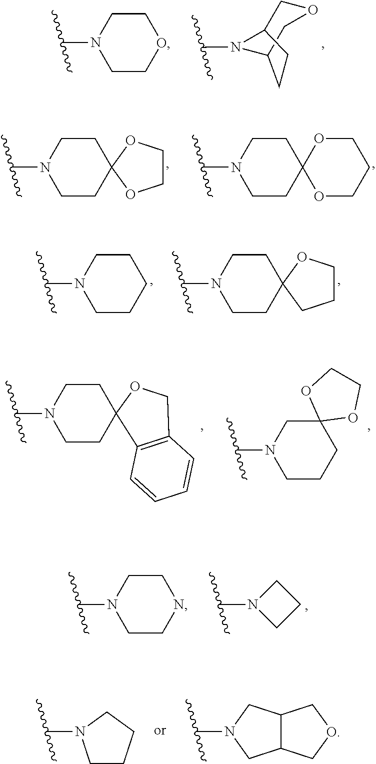
which is substituted or unsubstituted.

10. The compound of any one of claims 4 to 8, wherein R^{2A} and R^{2B} together with the nitrogen attached thereto form a

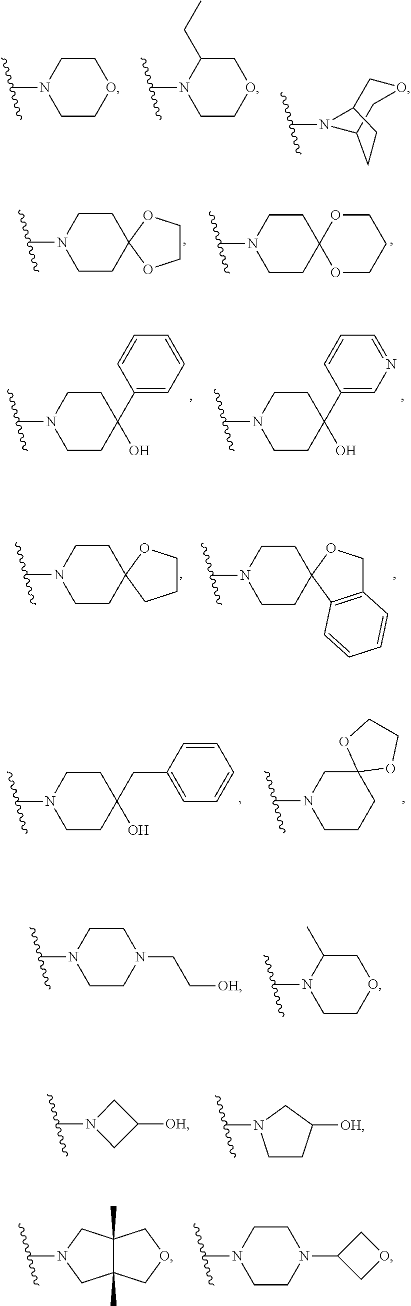

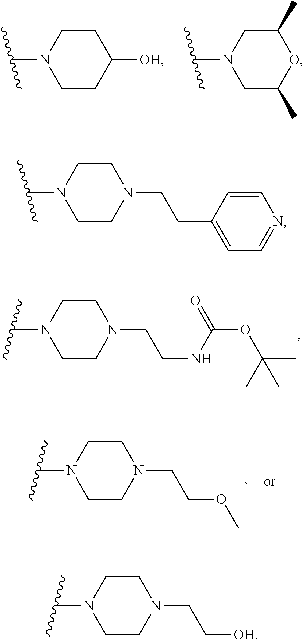

11. The compound of any one of claims 4 to 8, wherein one of R^{2A} and R^{2B} is hydrogen and the other one of R^{2A} and R^{2B} is

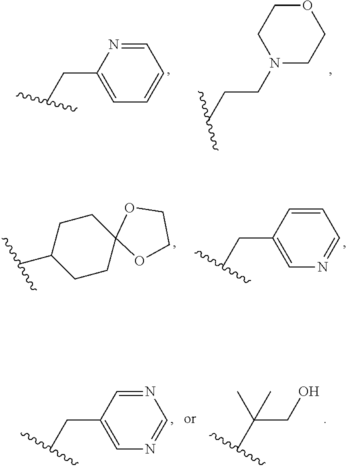

12. The compound of any one of claims 4 to 8, wherein R^{2A} and R^{2B} together with the nitrogen attached thereto form a 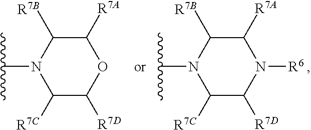
wherein R⁶ is hydrogen, halogen, —CX⁶₃, —CHX⁶₂, —CH₂X⁶, —OCX⁶₃, —OCH₂X⁶, —OCHX⁶₂, —CN, —OR^{6A}, substituted or unsubstituted alkyl, substituted or unsubstituted heteroalkyl, substituted or unsubstituted cycloalkyl, substituted or unsubstituted heterocycloalkyl;
X⁶ is —F, —Br, —Cl, or —I; and
Each R^{6A}, R^{7A}, R^{7B}, R^{7C} and R^{7D} is independently hydrogen, or substituted or unsubstituted alkyl.

13. The compound of claim 12, wherein R⁶ is —H,

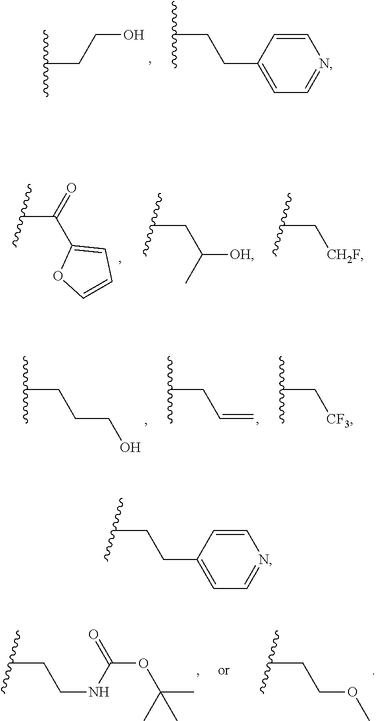

14. The compound of any one of claims 4 to 13, wherein R¹ is —CH₃, —OCF₃, —CF₃, —OCH₃, —CN or

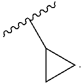 [SMILES: *C1CC1]

15. The compound of any one of claims 6 to 14, wherein each R^{10A}, R^{10B} and R^{10C} is independently hydrogen, halogen, or —CH₃.

16. The compound of any one of claims 1 to 3, wherein the compound has the structure of Formula (XII), 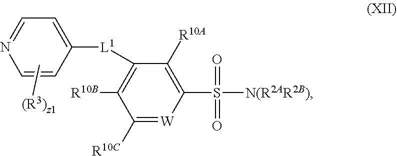
wherein:
L¹ is a bond or —NH—(CH₂)ₙ—;
n is an integer of 1 to 3;
z1 is an integer of 0 to 4;
R³ is independently hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
Each R^{10A}, R^{10B}, and R^{10C} is independently hydrogen, halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl.

17. The compound of claim 16, wherein R^{2A} and R^{2B} together with the nitrogen attached thereto form a 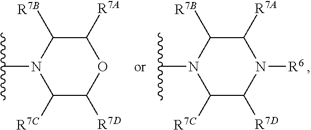
wherein R⁶ is hydrogen, halogen, —CX⁶₃, —CHX⁶₂, —CH₂X⁶, —OCX⁶₃, —OCH₂X⁶, —OCHX⁶₂, —CN, —OR^{6A}, substituted or unsubstituted alkyl, substituted or unsubstituted heteroalkyl, substituted or unsubstituted cycloalkyl, substituted or unsubstituted heterocycloalkyl;
X⁶ is —F, —Br, —Cl, or —I; and
Each R^{6A}, R^{7A}, R^{7B}, R^{7C} and R^{7D} is independently hydrogen, or substituted or unsubstituted alkyl.

18. The compound of claim 17, wherein R^{2A} and R^{2B} together with the nitrogen attached thereto form a

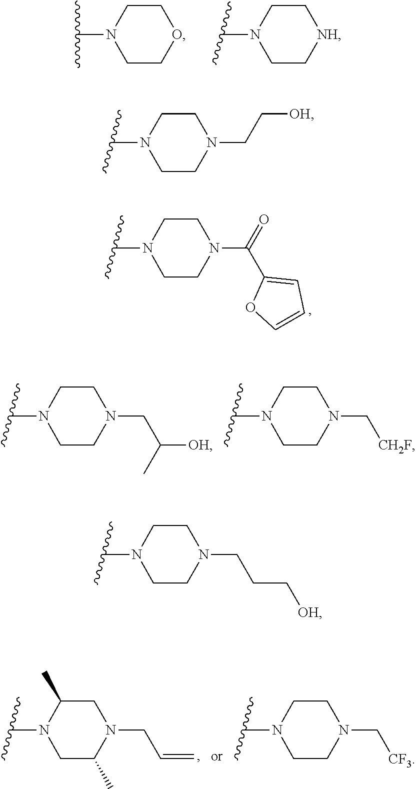

19. The compound of claim 17, wherein the compound has the structure of Formula (XII-a) or (XII-b),

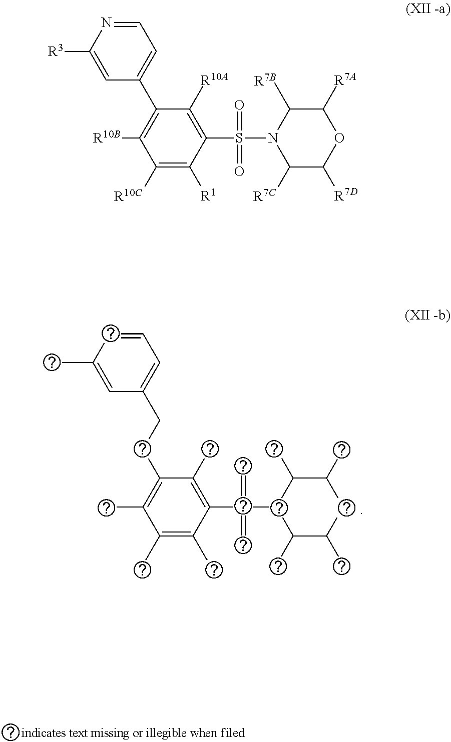

20. The compound of claim 17, wherein the compound has the structure of Formula (XII-c),

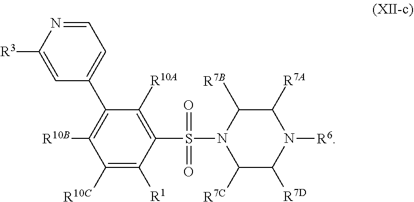

21. The compound of claim 20, wherein R⁶ is —H,

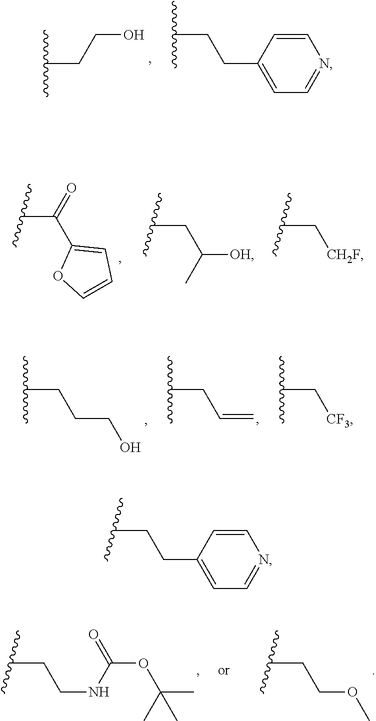

22. The compound of any one of claims 16 to 21, wherein R¹ is —CH₃.

23. The compound of any one of claims 16 to 22, wherein each R^{6A}, R^{7A}, R^{7B}, R^{7C} and R^{7D} is independently hydrogen, or —CH₃.

24. The compound of any one of claims 16 to 23, wherein two of R^{7A}, R^{7B}, R^{7C} and R^{7D} are independently hydrogen and the other two are —CH₃.

25. The compound of any one of claims 16 to 22, wherein R³ is hydrogen, halogen, substituted unsubstituted pyridyl, substituted or unsubstituted morphorinyl, substituted or unsubstituted phenyl, substituted or unsubstituted 2-6 membered heteroalkyl.

26. The compound of any one of claims 16 to 25, wherein R³ is hydrogen, halogen,

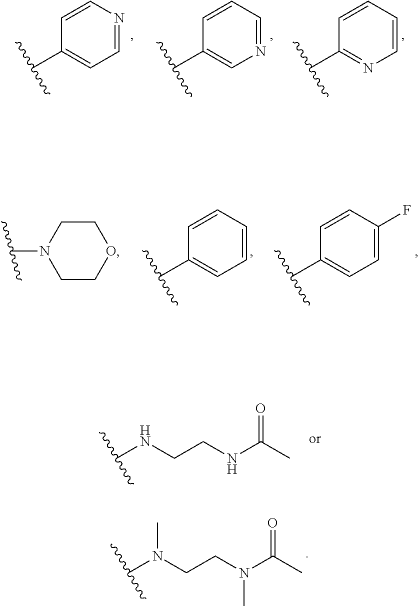

27. The compound of any one of claims 6 to 26, wherein each R^{10A}, R^{10B} and R^{10C} is independently hydrogen, halogen, or —CH₃.

28. The compound of any one of claims 1 to 3, wherein the compound has the structure of Formula (XIII), 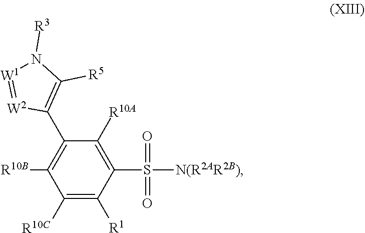
wherein:
W¹ is a —N═ or —CH═;
W² is a —N═ or —CR⁴—;
Each R³ and R⁵ is independently hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
Each R^{10A}, R^{10B}, and R^{10C} is independently hydrogen, halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl,
provided that when R¹ is hydrogen, then R^{2A} and R^{2B} together with the nitrogen atom form a substituted or unsubstituted heterocycloalkyl, or substituted or unsubstituted heteroaryl.

29. The compound of claim 28, wherein R^{2A} and R^{2B} together with the nitrogen attached thereto form a 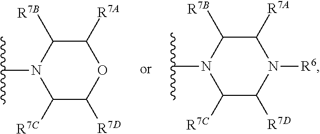
wherein R⁶ is hydrogen, halogen, —CX⁶₃, —CHX⁶₂, —CH₂X⁶, —OCX⁶₃, —OCH₂X⁶, —OCHX⁶₂, —CN, —OR^{6A}, substituted or unsubstituted alkyl, substituted or unsubstituted heteroalkyl, substituted or unsubstituted cycloalkyl, substituted or unsubstituted heterocycloalkyl;
X⁶ is —F, —Br, —Cl, or —I; and
Each R^{6A}, R^{7A}, R^{7B}, R^{7C} and R^{7D} is independently hydrogen, or substituted or unsubstituted alkyl.

30. The compound of any one of claims 28 to 29, wherein the compound has the Formula (XIII-a), (XIII-b), or (XIII-c),

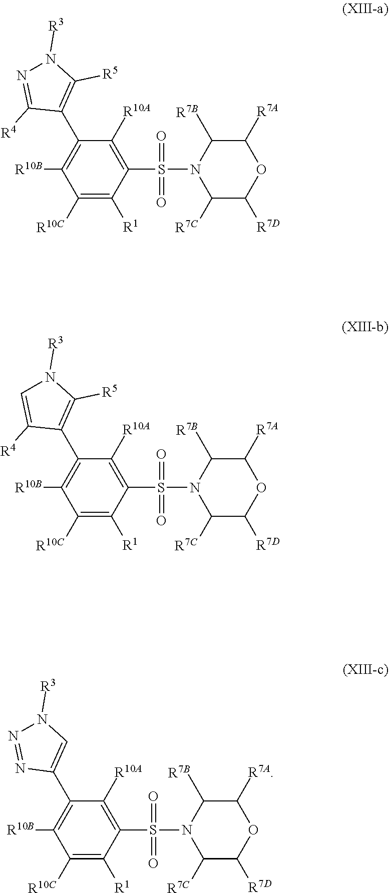

31. The compound of any one of claims 28 to 29, wherein the compound has the Formula (XIII-d), (XIII-e), or (XIII-f),

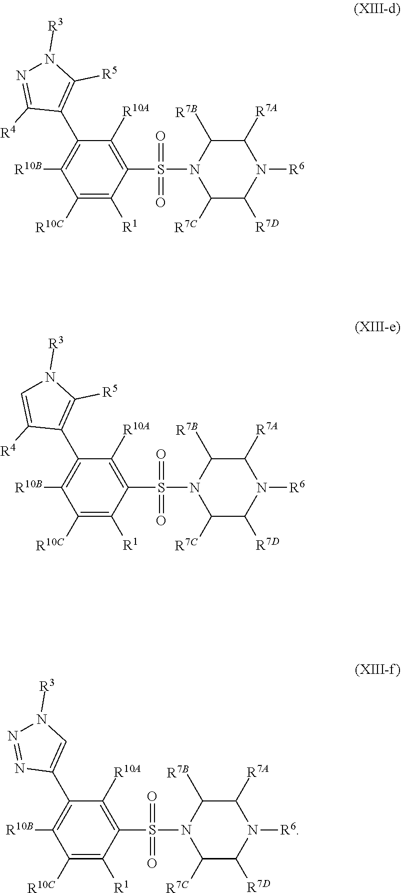

32. The compound of any one of claims 28 to 31, wherein:
R³ and R⁵ are hydrogen, and R⁴ is 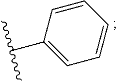 [SMILES: *c1ccccc1]
R⁴ and R⁵ are hydrogen, and R³ is —CH₃, 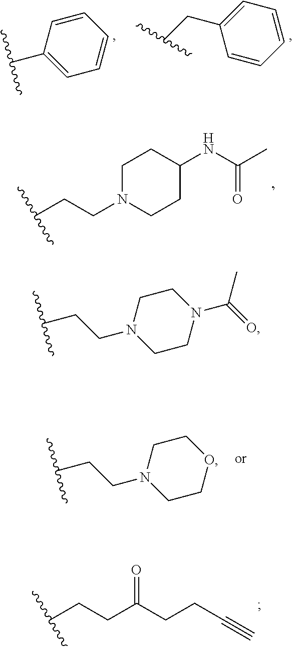
R³, R⁴ and R⁵ are hydrogen or —CH₃.

33. The compound of any one of claims 28 to 32, wherein R⁶ is —H,

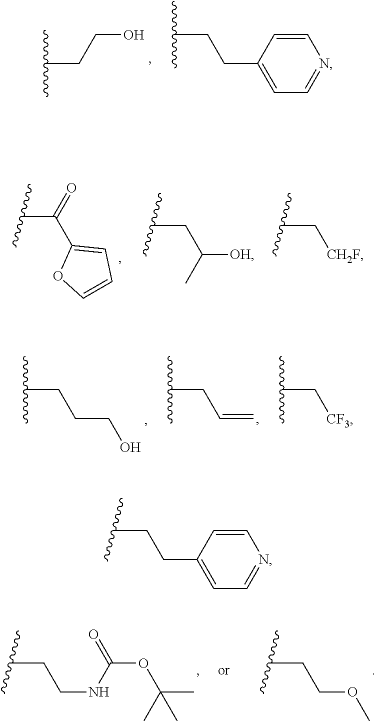

34. The compound of any one of claims 28 to 33, wherein R¹ is —CH₃.

35. The compound of any one of claims 28 to 34, wherein each R^{10A}, R^{10B} and R^{10C} is independently hydrogen, halogen, or —CH₃.

36. The compound of any one of claims 1 to 3, wherein the compound has the structure of Formula (XIV) or (XV), 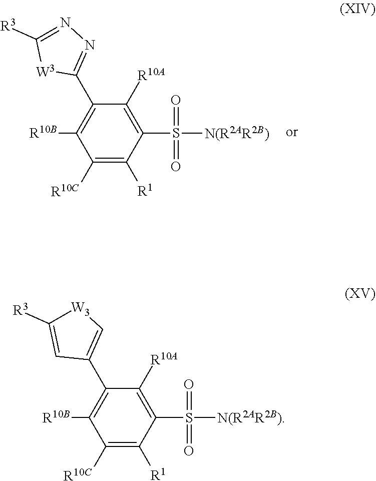
wherein:
W³ is a —S— or —O—;
R³ is hydrogen, halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; and
Each R^{10A}, R^{10B}, and R^{10C} is independently hydrogen, halogen, —CX¹₃, —CHX¹₂, —CH₂X¹, —OCX¹₃, —OCH₂X¹, —OCHX¹₂, —CN, —OR^{1A}, substituted or unsubstituted alkyl, substituted or unsubstituted heteroalkyl, substituted or unsubstituted cycloalkyl, substituted or unsubstituted heterocycloalkyl.

37. The compound of claim 36, wherein R^{2A} and R^{2B} together with the nitrogen attached thereto form a 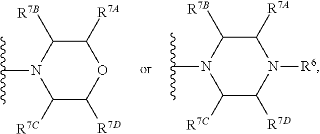
wherein R⁶ is hydrogen, halogen, —CX⁶₃, —CHX⁶₂, —CH₂X⁶, —OCX⁶₃, —OCH₂X⁶, —OCHX⁶₂, —CN, —OR^{6A}, substituted or unsubstituted alkyl, substituted or unsubstituted heteroalkyl, substituted or unsubstituted cycloalkyl, substituted or unsubstituted heterocycloalkyl;
X⁶ is —F, —Br, —Cl, or —I; and
Each R^{6A}, R^{7A}, R^{7B}, R^{7C} and R^{7D} is independently hydrogen, or substituted or unsubstituted alkyl.

38. The compound of any one of claims 36 to 37, wherein the compound has the structure of Formula (XIV-a), (XIV-b), (XV-a), or (XV-b),

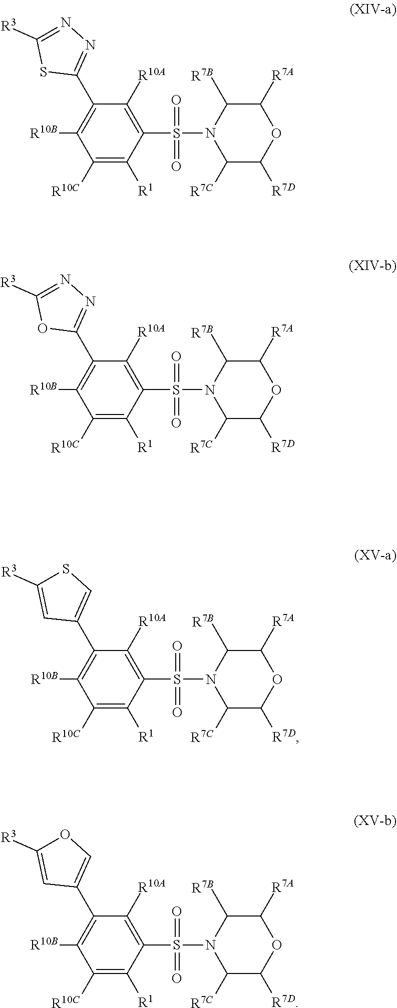

39. The compound of any one of claims 36 to 38, wherein R³ is

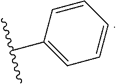 [SMILES: *c1ccccc1]

40. The compound of any one of claims 36 to 39, wherein R¹ is —CH₃.

41. The compound of any one of claims 36 to 40, wherein each R^{10A}, R^{10B} and R^{10C} is independently hydrogen, halogen, or —CH₃.

42. The compound of any one of claims 1 to 3, wherein the Ring A is selected from 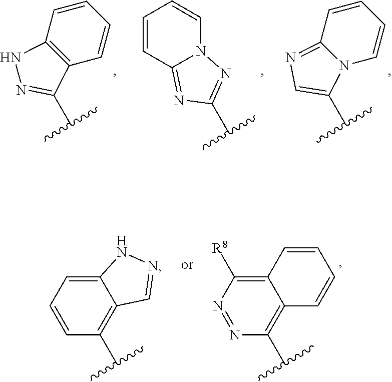
wherein R⁸ is hydrogen, or substituted or unsubstituted alkyl;
the Ring A is unsubstituted or substituted with one or more R³; and
R³ is independently halogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl;
provided that when Ring A is unsubstituted 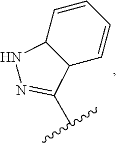 [SMILES: *C1=NNC2C=CC=CC12]
—N(R^{2A}R^{2B}) is not a 4-substituted piperidinyl.

43. The compound of claim 42, wherein R^{2A} and R^{2B} together with the nitrogen attached thereto form a 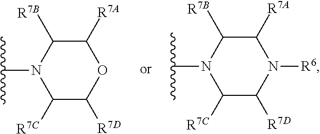
wherein R⁶ is hydrogen, halogen, —CX⁶₃, —CHX⁶₂, —CH₂X⁶, —OCX⁶₃, —OCH₂X⁶, —OCHX⁶₂, —CN, —OR^{6A}, substituted or unsubstituted alkyl, substituted or unsubstituted heteroalkyl, substituted or unsubstituted cycloalkyl, substituted or unsubstituted heterocycloalkyl;
X⁶ is —F, —Br, —Cl, or —I; and
Each R^{6A}, R^{7A}, R^{7B}, R^{7C} and R^{7D} is independently hydrogen, or substituted or unsubstituted alkyl.

44. The compound of any of claims 42 to 43, wherein R¹ is —CH₃.

45. The compound of any one of claims 42 to 44, wherein each R^{10A}, R^{10B} and R^{10C} is independently hydrogen, halogen, or —CH₃.

46. The compound of any one of claims 1 to 44, wherein the compound is any compound in Tables 1 to 5.

47. A pharmaceutical composition comprising a compound of any one of claims 1 to 46, a pharmaceutically acceptable salt form thereof, an isomer thereof, or a crystal form thereof.

48. A method of inhibiting NAD consumption and/or increasing NAD synthesis in a patient, comprising administering to the patient an effective dose of a compound of any one of claim 1 to 46.

49. The methods of claim 48, wherein increasing NAD synthesis is achieved by activating the enzyme nicotinamide phosphoribosyltransferase.

50. A method of preventing or inhibiting NAD depletion in a patient, or a method of improving a condition linked to alterations of NAD metabolism in a patient, comprising administering to the patient an effective dose of a compound of any one of claim 1 to 46.

51. A method of providing protection from toxicity of misfolded proteins in a patient, comprising administering to the patient an effective dose of a compound of any one of claim 1 to 46.

52. A method of preventing or treating a degenerative disease in a patient, comprising administering to the patient an effective dose of a compound of any one of claim 1 to 46.

53. The methods of claim 52, wherein the degenerative disease is a a peripheral amyloidosis or a neurodegenerative disorder associated with misfolded protein-induced neurodegeneration and/or NAD depletion.

54. The methods of claim 52, wherein the degenerative disease is Creutzfeldt-Jakob Disease or other prion disease, Parkinson's disease, dementia with Lewy bodies, multiple system atrophy or other synucleinopathy, Alzheimer's disease, amyotrophic lateral sclerosis, fronto-temporal dementia or other tauopathy, multiple sclerosis, chronic traumatic encephalopathy, ATTR, brain ischemia or an axonopathy.

55. A method of preventing or treating a retinal disease in a patient, comprising administering to the patient an effective dose of a compound of any one of claim 1 to 46.

56. A method of preventing or treating diabetes, non alcoholic fatty liver disease or other metabolic disease in a patient, comprising administering to the patient an effective dose of a compound of any one of claim 1 to 46.

57. A method of preventing or treating a kidney disease in a patient, comprising administering to the patient an effective dose of a compound of any one of claim 1 to 46.

58. A method of mitigating health effects of aging, comprising administering to the patient an effective dose of a compound of any one of claim 1 to 46.
